(19) United States
(12) Patent Application Publication
(10) Pub. No.: US 2023/0279194 A1
(43) Pub. Date: Sep. 7, 2023

(54) PROCESS FOR CATALYTIC UPCYCLING OF HYDROCARBON POLYMERS TO ALKYLAROMATIC COMPOUNDS

(71) Applicant: The Regents of the University of California, Oakland, CA (US)

(72) Inventors: Mahdi M. Abu-Omar, Santa Barbara, CA (US); Manhao Zeng, Santa Barbara, CA (US); Susannah Scott, Santa Barbara, CA (US); Fan Zhang, Santa Barbara, CA (US); Jiakai Sun, Santa Barbara, CA (US); Yu-Hsuan Lee, Santa Barbara, CA (US)

(21) Appl. No.: 18/005,830

(22) PCT Filed: Jul. 15, 2021
(86) PCT No.: PCT/US2021/041812

§ 371 Date: Jan. 17, 2023

Related U.S. Application Data
(60) Provisional application No. 63/052,227, filed on Jul. 15, 2020.

(51) Int. Cl. C08J 11/16; C07C 4/18

(52) U.S. Cl. CPC C08J 11/16; C07C 4/18; C07C 2521/04; C07C 2523/42; C08J 2323/06

(57) ABSTRACT

Process for upcycling a waste material to form alkylaromatic compounds is described herein. The process typically includes the steps of feeding a waste material containing hydrocarbon polymer(s) into a reactor containing a catalyst therein, and operating the reactor at a sufficient temperature for a sufficient period of time to convert the hydrocarbon polymer(s) to a liquid and/or wax product containing alkylaromatic compound(s). Each of the alkylaromatic compound(s) contains at least 10 carbon atoms. The catalyst contains a transition metal or a mixture of a transition metal and another metal. Optionally, the catalyst is dispersed on the surface of a support. The product may contain other unsaturated compounds, such as olefins. Typically, the reactor operates at a temperature in the range between 250° C. and 350° C. The total selectivity of the process to form the one or more alkylaromatic compounds is typically between 50 mol % and 95 mol %.

### CROSS-REFERENCE TO RELATED APPLICATIONS

This application claims the benefit of and priority to U.S. Provisional Application No. 63/052,227 filed Jul. 15, 2020, which is hereby incorporated by reference in its entirety.

### STATEMENT REGARDING FEDERALLY SPONSORED RESEARCH OR DEVELOPMENT

This invention was made with government support under DE-AC02-07CH11358 awarded by the United States Department of Energy. The government has certain rights in the invention.

### FIELD OF THE INVENTION

The invention is generally in the field of upcycling of hydrocarbon polymers; particularly methods for upcycling of waste hydrocarbon polymers.

### BACKGROUND OF THE INVENTION

Over the past 70 years, global production of synthetic, petroleum-based plastics has risen sharply, from less than 2 Mt in 1950 to 359 Mt in 2018 (PlasticsEurope, “Plastics—The facts 2019: An analysis of European plastics production, demand and waste data”, 2019). Production is projected to double again in the coming decade (Pravettoni, United Nations Environment Programme, GRID-Arendal, 2018). Plastics have become indispensable in many facets of modern life, enhancing the security of our food and health care systems, the performance of textiles, the versatility of consumer electronics, and the energy efficiency of transportation. About 40% of these plastics are destined for short-term use, and most (in the US: >90%) are not recycled (Geyer, et al., Sci. Adv. 3, e1700782 (2017)). The vast bulk of this plastic waste ends up in landfills or being incinerated. However, the embodied energy that can be recovered by combustion is far less than that used in the original manufacturing of the plastic (Eriksson, et al., Energ. Environ. Sci. 2, 907-914 (2009)). Furthermore, a significant fraction of the waste is mismanaged, ending up in rivers and oceans, where its chemical inertness leads to extremely slow degradation and visible accumulation in the natural environment (Jambeck, et al., Science 347, 768-771 (2015); Chamas, et al., ACS Sustain. Chem. Eng. 8, 3494-3511 (2020)).

Efforts to develop closed-loop lifecycles for synthetic plastics by relying on collection, separation, and mechanical recycling have had limited success. The inferior properties of the recycled materials, relative to virgin plastics, contribute to the economic challenges of the “downcycling” model (Rahimi, et al., Nat. Rev. Chem. 1, 0046 (2017)). New types of polymers that degrade rapidly in the environment are being investigated (Zhu, et al., Nature 540, 354-362 (2016)), although such materials do not currently have either the physical properties or the cost structure to displace existing commodity plastics. Degradable plastics can also contaminate recycling streams, and may encourage single-use product design. Depolymerization (also known as chemical or feedstock recycling) can recover the original monomer subunits whose subsequent repolymerization gives materials with properties identical to those of the original plastic (Vollmer, et al., Angew. Chem. Int. Ed., DOI:10.1002/anie.201915651 (2020)). However, this strategy requires prohibitive amounts of energy for polyolefins, such as polyethylene (PE) and polypropylene (PP). Controlled partial depolymerization could convert post-consumer waste plastics directly into more valuable chemicals (“upcycling”), although few such processes have yet been developed.

High and low density polyethylenes currently represent the largest fraction (36% by mass) of all plastic waste (Geyer, et al., Sci. Adv. 3, e1700782 (2017)). Their depolymerization by pyrolysis at temperatures above 400° C., with or without a catalyst, generates complex, low-value mixtures of gas, liquid hydrocarbons and char (Ignatyev, et al., ChemSusChem 7, 1579-1593 (2014); Sharuddin, et al., Energ. Convers. Manage. 115, 308-326 (2016)). US 2020/0017772 by Ramamurthy, et al. discloses a process for producing propylene and cumene from plastics via a complex multi-unit operation. Somewhat more selective disassembly can be achieved at lower temperatures via catalytic hydrogenolysis (Dufaud, et al., Angew. Chem. Int. Ed. 37, 806-810 (1998); Celik, et al., ACS Cent. Sci. 5, 1795-1803 (2019)) or tandem catalytic alkane metathesis (Jia, et al., Sci. Adv. 2, e1501591 (2016)), but the low-value alkane products are unlikely to recoup the costs of recovery, separation, and processing using large amounts of a co-reactant (H₂ or liquid alkanes, respectively).

Aromatics are more attractive target products from partial depolymerization. The conventional process for making aromatics is naphtha reforming. This energy-intensive process generates a mixture known as benzene-toluene-xylenes (BTX) at 500-600° C. (Rahimpour, et al., Appl. Energ. 109, 79-93 (2013)). In a subsequent step with a large environmental footprint, BTX is alkylated to give linear alkylbenzenes (LAB), which can be used to make surfactants. The most widely-used processes require linear olefins (typically, C₁₀-C₁₆) and liquid HF or AlCl₃—HCl as the acid catalyst (Perego, et al., Catal. Today 73, 3-22 (2002)). Manufacturing BTX by aromatization of shale gas-derived light alkanes requires harsher reaction conditions (propane: 550-700° C.; ethane: 600-800° C.; methane: 900-1000° C.) (Kanitkar, et al., Natural Gas Processing from Midstream to Downstream, Wiley, pp. 379-401, 2019), and the catalysts tend to deactivate rapidly. New zeolite-based catalysts can use either methanol (Yarulina, et al., Nat. Catal. 1, 398-411 (2018)), or syngas (Cheng, et al., Chem 3, 334-347 (2017)), to make BTX aromatics at lower temperatures, 300-400° C. Biomass-based routes include oxidative coupling of ethanol to aromatic alcohols and aldehydes (Wang, et al., ACS Catal. 9, 7204-7216 (2019)), hydrogenolysis/hydrodeoxygenation of bio-oils or lignin to give propylbenzene (Maneffa, et al., ChemSusChem. 9, 2736-2748 (2016)), and Diels-Alder reactions of carbohydrate-derived furanics to give p-xylene (Williams, et al., ACS Catal. 2, 935-939 (2012)). However, slow rates, low yields, and high H₂ requirements would make them expensive to operate and none are practiced commercially. BTX is also formed in the catalytic pyrolysis of PE at 400-600° C., although deactivation of the zeolite catalysts by coking is severe (Lopez, et al., Renew. Sust. Energ. Rev. 73, 346-368 (2017)). The yields are moderate (up to 50 wt %), and large amounts of low-value gases (C₁ to C₅, >50 wt %) are formed.

There remains a need for improved processes for upcycling hydrocarbon polymers.

Therefore, it is the object of the present invention to provide a process for upcycling plastic waste containing hydrocarbon polymers to more valuable materials, such as alkylaromatic compounds.

It is a further object of the present invention to provide an improved depolymerization process to produce more valuable materials.

It is a further object of the present invention to provide compositions formed from depolymerization processes.

It is a further object of the present invention to provide compositions formed from upcycling plastic waste.

### SUMMARY OF THE INVENTION

Processes for upcycling plastic waste containing one or more hydrocarbon polymers to alkylaromatic compounds are described herein. The process typically includes (i) feeding a plastic waste containing a hydrocarbon polymer, optionally more than one hydrocarbon polymer, into a reactor and (ii) operating the reactor at a sufficient temperature for a sufficient period of time to convert the hydrocarbon polymer(s) to a product in the form of a liquid and/or wax that contains an alkylaromatic compound, such as a dialkylbenzene, optionally more than one alkylaromatic compound. Typically, the mol % of the alkylaromatic compound, optionally the total mol % of the more than one alkylaromatic compound, in the product (i.e. selectivity) is at least 50 mol %, such as between 50 mol % and 95 mol %.

The reactor contains a catalyst therein. The catalyst includes a transition metal. Typically, the catalyst is in the form of atoms, nanoclusters, or nanoparticles, or a combination thereof. Optionally, the catalyst is dispersed in the form of atoms, nanocluster, or nanoparticles, or a combination thereof on the surface of a support.

Typically, the reactor operates at a temperature of up to 500° C. or up to 350° C., such as between 250° C. and 300° C.

### BRIEF DESCRIPTION OF THE DRAWINGS

FIGS. 1A-1B are graphs showing a histogram of fresh Pt/γ-Al₂O₃ cluster size distribution before reaction (FIG. 1A) and after reaction (FIG. 1B).

FIG. 2 is a graph showing IR spectra of solid residue (including the catalyst and KBr) recovered after PE reaction for 3 h and 24 h.

FIG. 3 is a bar graph showing hydrocarbon distributions of solvent-free conversion of various types of PE after 24 h at 280° C. in five separate experiments. Reactions of a low molecular weight PE (M_{w}=3.52×10³ g mol⁻¹, Ð=1.90) in an unstirred mini-autoclave reactor: (0) catalyzed by γ-Al₂O₃ (no gas recovery) or (1) catalyzed by Pt/γ-Al₂O₃ (no gas recovery); (2) catalyzed by Pt/γ-Al₂O₃ (with gas recovery). Reactions catalyzed by Pt/γ-Al₂O₃ in a stirred autoclave reactor, with gas recovery: (3) low molecular weight PE; (4) LDPE bag (M_{w}=9.45×10⁴ g mol⁻¹, Ð=7.37); and (5) HDPE bottle cap (M_{w}=5.35×10⁴ g mol⁻¹, Ð=3.61).

FIG. 4 is a graph showing GPC analysis of a liquid hydrocarbon fraction from the solvent-free catalytic conversion of polyethylene (M_{w}=3.52×10³ g mol⁻¹) after 24 h at 280° C. (Exp. 3) conducted using both RI and UV detectors.

FIG. 5 shows possible structures for mono-, di- and tri-substituted alkylbenzenes, and their corresponding H_{α}/Hₐᵣ ratios. Only —C_{α}H₂R and —C_{α}H₃ substituents are considered, due to their intense ¹H NMR signals observed at 2.35-2.85 ppm.

FIG. 6 is a schematic illustrating the overall PE conversion to alkylaromatics and alkylnaphthenes, proposed mechanism of tandem polyethylene hydrogenolysis/aromatization via dehydrocyclization, and the estimated yields of each kind of product estimated using a combination of ¹H NMR and FD-MS.

FIGS. 7A-7D are graphs showing log-normal distribution fit of the major hydrocarbon mass series observed by FD-MS: alkylbenzenes (FIG. 7A), cycloalkanes (FIG. 7B), alkanes and alkylnaphthalenes (FIG. 7C), and alkyltetralins (FIG. 7D).

FIG. 8 is a graph showing GPC and molecular weight distribution of lighter and heavier fractions of liquid/wax products from the catalytic disassembly of PE (M_{w}=3.52×10³ g mol⁻¹, Ð=1.9, average C₁₃₂), after heating with 1.5 wt % Pt/γ-Al₂O₃ for 36 h at 280° C. in Parr reactor.

FIGS. 9A-9D are graphs showing the time course of the solvent-free disassembly of polyethylene (Mₙ=1.85×10³ g mol⁻¹; Ð=1.90) catalyzed by Pt/γ-Al₂O₃ in an unstirred mini-autoclave reactor at 280° C.: evolution of major product fractions (top portions: CHCl₃-soluble liquids/waxes; bottom portions insoluble hydrocarbons) (FIG. 9A); and overall molecular weight (Mₙ, solid line) and dispersity (Ð, dashed line) for all non-gas hydrocarbons (FIG. 9B). The curvefit (solid line) shows the refinement of eq 3 to the Mₙ data. Initial conditions: total carbon n_{C}=8.4 mmol; number of polymer chains N₀=68 μmol; total Pt m_{Pt}=3×10⁻³ g; selectivity for aromatization vs. hydrogenolysis, s=1/2 (eq S19). Time course of the ¹H NMR spectra of the liquid/wax fraction, in the aromatic region (* indicates a truncated residual solvent signal) (FIG. 9C); and the fraction of aromatic protons, and the ratio of mono- to poly-aromatic protons (FIG. 9D).

FIG. 10 is a bar graph showing the distribution of products and unreacted starting material from the catalytic reaction of C₃₀H₆₂ with 1.5 wt % Pt/γ-Al₂O₃ in a 12.8 mL mini-autoclave at 280° C. for 24 h.

FIG. 11 is a graph showing the fraction of aliphatic carbon, n_{A}/n_{C}, as a function of the dimensionless time τ=6skm_{Pt} t/Kn_{C}, assuming K>>1 and no aromatics initially present in the reactor, given by eq S26.

FIG. 12 is graph showing the relationship between inverse molecular weight and dimensionless time τ=6skm_{Pt} t/Kn_{C}, given by eq 531, when s=0.5.

FIG. 13 is a graph showing IR spectra in the wavenumber range of 1000 cm⁻¹-4000 cm⁻¹ of solid residue (containing the catalyst and diluted with KBr) after PE upgrading reaction at 280° C. for 24 h (dashed line) and further calcination in O₂ at 400° C. (solid line), respectively.

FIG. 14 is a scheme illustrating an exemplary process for catalytic upcycling of plastic waste containing hydrocarbon polymers to alkylaromatic compounds.

### DETAILED DESCRIPTION OF THE INVENTION

### I. Process for Depolymerizing a Waste Solid Waste

Process for upcycling a plastic waste material is described herein. Generally, the term “upcycling” refers to the depolymerization of a waste material to more valuable chemicals, such as aromatic compounds. The waste contains a hydrocarbon polymer, optionally more than one hydrocarbon polymer. In the process, the hydrocarbon polymer in the waste is generally upcycled to form one or more aromatic compounds, such as one or more alkylaromatic compounds.

The process can also be a process for manufacturing aromatic compounds, such as alkylaromatic compounds.

The upcycling process includes a depolymerization reaction. Optionally, the depolymerization reaction is solvent-free or hydrogen gas-free, or a combination thereof. Optionally, the depolymerization reaction is hydrogen gas-free. For example, no hydrogen gas is added to the reactor for the depolymerization reaction. Optionally, the process is solvent-free. For example, the waste material is in a solid form and is not dissolved in a solvent prior to or during the depolymerization reaction. Generally, the depolymerization reaction is performed at a temperature≤500° C., optionally at a temperature≤350° C.

The process generally includes (i) feeding the waste material containing hydrocarbon polymer(s) into a reactor, where the reactor contains a catalyst therein; and (ii) operating the reactor at a sufficient temperature for a sufficient period of time to convert the hydrocarbon polymer(s) to a product.

The product contains an alkylaromatic compound, optionally more than one alkylaromatic compound, and is generally in the form of a liquid, a wax, or a combination thereof. Typically, the mol % of the alkylaromatic compound, optionally the total mol % of the more than one alkylaromatic compound, in the product is at least 50 mol %, such as between 50 mol % and 95 mol %. The mole % of the alkylaromatic compound or the total mole % of the more than one alkylaromatic compound in the product is also referred herein as “the selectivity to alkylaromatic compound”, when one alkylaromatic compound is present in the product, or “the total selectivity to alkylaromatic compounds”, when more than one alkylaromatic compound is present in the product.

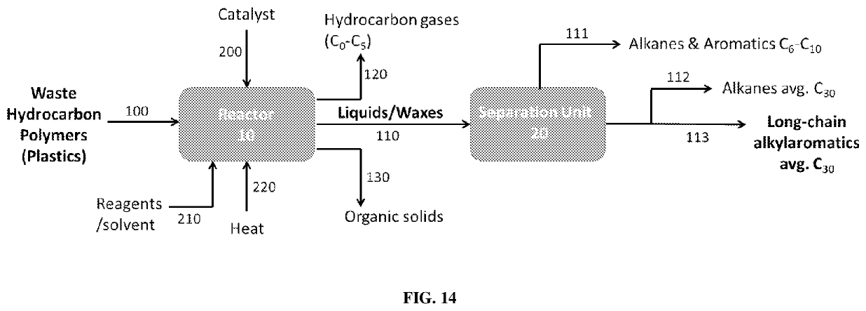

An exemplary process for upcycling a waste material is illustrated in FIG. 14 and described below.

Without being bound by theory, it is believed that hydrogenolysis and aromatization via dehydrocyclization occur in tandem, i.e. together, during this reaction to covert polyethylene to the resulting liquid and/or wax product.

Optionally, the process does not include a hydrocracking step, an olefin metathesis step, or a benzene alkylation step, or a combination more than one of these steps.

A. Feed a Waste Material into a Reactor.

Generally, the waste material is fed into the reactor. Optionally, the waste material is in the form of a solid. The waste material contains a plastic, optionally a mixed plastics. Examples of suitable plastics that can be upcycled using the process include, but are not limited to, non-chlorinated plastics, such as polyolefins, polyethylene, polypropylene, polystyrene, and copolymers thereof; and chlorinated plastics, such as polyvinylchloride (PVC), polyvinylidene chloride (PVDC), etc. In the reactor, the waste material is in contact with the catalyst in the reactor. Reactors that can be used to upcycle a plastic waste material are known. For example, the reactor can be an autoclave reactor, such as a pressure reactor system, a flow reactor, a fixed-bed reactor, a packed-bed reactor (PBR), a continuous stirred tank reactor (CSTR), or a semi-batch reactor.

1. Hydrocarbon Polymers

The waste material contains a hydrocarbon polymer. The hydrocarbon polymer in the waste can be polyethylene, polypropylene, polystyrene, a copolymer of polyethylene, a copolymer of polypropylene, a copolymer of polyethylene and polypropylene, or acrylonitrile butadiene styrene (ABS). For example, the waste material contains polyethylene or polypropylene. Optionally, the waste material contains more than one hydrocarbon polymer described above, i.e. a mixture of two or more hydrocarbon polymers described above. For example, the waste material contains a mixture of polyethylene and polypropylene.

Optionally, the waste material contains a hydrocarbon polymer, which is a mixture of different densities of the same hydrocarbon polymer. For example, the waste material can contain polyethylene, which is a mixture of high density polyethylene (HDPE) and low density polyethylene (LDPE).

LDPE generally has a density in the range of 917 to 930 kg/m³. It is not reactive at room temperatures, except by strong oxidizing agents, and some solvents cause swelling. LDPE has more branching (on about 2% of the carbon atoms) than HDPE.

The density of HDPE generally is in the range of 930 to 970 kg/m³. Although the density of HDPE is only marginally higher than that of low-density polyethylene, HDPE has little branching, giving it stronger intermolecular forces and tensile strength than LDPE. The difference in strength between HDPE and LDPE exceeds the difference in density, giving HDPE a higher specific strength (a material's strength (force per unit area at failure) divided by its density; also known as the strength-to-weight ratio).

Optionally, the waste material contains a hydrocarbon polymer of different molecular weights (weight average molecular weight Mw or number average molecular weight Mn). For example, the waste material contains polyethylene, which is a mixture of high molecular weight polyethylene and medium molecular weight polyethylene, a mixture of high molecular weight polyethylene and low molecular weight polyethylene, a mixture of medium molecular weight polyethylene and low molecular weight polyethylene, or a mixture of high, medium, and low molecular weight polyethylene. Generally, a hydrocarbon polymer of low Mw is in the range from 1000 g mol⁻¹ to 10,000 g mol⁻¹; a hydrocarbon polymer of medium Mw is in the range from 10,000 g mol⁻¹ to 200,000 g mol⁻¹; a hydrocarbon polymer of high Mw is in the range from 200,000 g mol⁻¹ to 500,000 g mol⁻¹; and a hydrocarbon polymer of ultrahigh Mw is in the range from 500,000 g mol⁻¹ to 7,500,000 g mol⁻¹.

a. Average Molecular Weight

Typically, the hydrocarbon polymer in the waste material has a weight average molecular weight (Mw) of at least 1×10³ g mol⁻¹, 2×10³ g mol⁻¹, at least 2.5×10³ g mol⁻¹, at least 3×10³ g mol⁻¹, at least 3.5×10³ g mol-, at least 4×10³ g mol⁻¹, up to 7.5×10⁶ g mol⁻¹, 5×10⁶ g mol⁻¹, 1×10⁶ g mol⁻¹, 5×10⁵ g mol⁻¹, up to 1×10⁵ g mol⁻¹, up to 9.5×10⁴ g mol⁻¹, up to 9×10⁴ g mol⁻¹, up to 8.5×10⁴ g mol⁻¹, up to 8×10⁴ g mol⁻¹, up to 7.5×10⁴ g mol⁻¹, up to 7×10⁴ g mol⁻¹, up to 6.5×10⁴ g mol⁻¹, up to 6×10⁴ g mol⁻¹, up to 5.5×10⁴ g mol⁻¹, up to 5×10⁴ g mol⁻¹, between 1×10³ g mol⁻¹ and 7.5×10⁶ g mol⁻¹, between 1×10³ g mol⁻¹ and 5×10⁶ g mol⁻¹, between 1×10³ g mol⁻¹ and 1×10⁶ g mol⁻¹, between 1×10³ g mol⁻¹ and 5×10⁵ g mol⁻¹, between 2×10³ g mol⁻¹ and 1×10⁵ g mol⁻¹, between 2×10³ g mol⁻¹ and 1×10⁵ g mol⁻¹, between 3×10³ g mol⁻¹ and 1×10⁵ g mol⁻¹, between 4×10³ g mol⁻¹ and 1×10⁵ g mol⁻¹, between 4×10³ g mol⁻¹ and 9.5×10⁴ g mol⁻¹, between 4×10³ g mol⁻¹ and 5.5×10⁴ g mol⁻¹, or between 4×10³ g mol⁻¹ and 5×10⁴ g mol⁻¹.

Optionally, the hydrocarbon polymer in the waste material has a number average molecular weight (Mn) of at least 1×10² g mol⁻¹, at least 5×10² g mol⁻¹, at least 1×10³ g mol⁻¹, at least 1.5×10³ g mol⁻¹, at least 2×10³ g mol⁻¹, at least 2.5×10³ g mol⁻¹, at least 3×10³ g mol⁻¹, at least 3.5×10³ g mol⁻¹, at least 4×10³ g mol⁻¹, up to 2×10⁴ g mol⁻¹, up to 1.5×10⁴ g mol⁻¹, up to 1×10⁴ g mol⁻¹, between 1×10³ g mol⁻¹ and 2×10⁴ g mol⁻¹, between 1.5×10³ g mol⁻¹ and 2×10⁴ g mol⁻¹, between 1.8×10³ g mol⁻¹ and 2×10⁴ g mol⁻¹, between 2×10³ g mol⁻¹ and 2×10⁴ g mol⁻¹, between 3×10³ g mol⁻¹ and 2×10⁴ g mol⁻¹, between 4×10³ g mol⁻¹ and 2×10⁴ g mol⁻¹, between 1.5×10³ g mol⁻¹ and 1.5×10⁴ g mol⁻¹, or between 1.8×10³ g mol⁻¹ and 1.5×10⁴ g mol⁻¹.

For example, the waste material contains a polyethylene having a Mw of at least 3×10³ g mol⁻¹ and a Mn of at least 1.5×10³ g mol⁻¹, such as a Mw in a range between 3×10³ g mol⁻¹ and 1×10⁵ g mol⁻¹ and a Mn in a range between 1.5×10³ g mol⁻¹ and 1.5×10⁴ g mol⁻¹, such as a Mw of 3.52×10³ g mol⁻¹ and a Mn of 1.85×10³ g mol⁻¹.

Optionally, the waste material contains a hydrocarbon polymer of different average molecular weights (also referred herein as “molecular weight”). Generally, a low molecular weight hydrocarbon polymer has a Mw in a range from 1000 g mol⁻¹ to 10,000 g mol⁻¹ and a Mn in a range from 100 g mol⁻¹ to 1,000 g mol⁻¹; a medium molecular weight hydrocarbon polymer has a Mw in a range from 10,000 g mol⁻¹ to 200,000 g mol⁻¹ and a Mn in a range from 1,000 g mol⁻¹ to 20,000 g mol⁻¹; a high molecular weight hydrocarbon polymer has a Mw in a range from 200,000 g mol⁻¹ to 500,000 g mol⁻¹ and a Mn in a range from 20,000 g mol⁻¹ to 50,000 g mol⁻¹; and an ultrahigh molecular weight hydrocarbon polymer has a Mw in a range from 500,000 g mol⁻¹ to 7,500,000 g mol⁻¹ and a Mn in a range from 50,000 g mol⁻¹ to 750,000 g mol⁻¹. For example, the waste material contains polyethylene, which is a mixture of high molecular weight polyethylene and medium molecular weight polyethylene, a mixture of high molecular weight polyethylene and low molecular weight polyethylene, a mixture of medium molecular weight polyethylene and low molecular weight polyethylene, or a mixture of high, medium, and low molecular weight polyethylene.

Recitation of ranges of values herein are merely intended to serve as a shorthand method of referring individually to each separate value falling within the range, unless otherwise indicated herein, and each separate value is incorporated into the specification as if it were individually recited herein.

b. Number of Carbon Atoms in Hydrocarbon Polymer

Typically, the hydrocarbon polymer(s) in the waste material contains at least 70 carbon atoms, at least 100 carbon atoms, at least 120 carbon atoms, at least 150 carbon atoms, at least 200 carbon atoms, at least 250 carbon atoms, at least 300 carbon atoms, at least 350 carbon atoms, at least 400 carbon atoms, at least 450 carbon atoms, at least 500 carbon atoms, at least 550 carbon atoms, at least 600 carbon atoms, at least 650 carbon atoms, at least 700 carbon atoms, at least 750 carbon atoms, at least 800 carbon atoms, at least 850 carbon atoms, at least 900 carbon atoms, at least 950 carbon atoms, at least 1000 carbon atoms, up to 500,000 carbon atoms, up to 100,000 carbon atoms, up to 50,000 carbon atoms, up to 10,000 carbon atoms, up to 7000 carbon atoms, up to 6500 carbon atoms, up to 6000 carbon atoms, up to 5500 carbon atoms, up to 5000 carbon atoms, up to 4500 carbon atoms, up to 4000 carbon atoms, up to 3500 carbon atoms, between 70 and 500,000 carbon atoms, between 100 and 100,000 carbon atoms, between 120 and 50,000 carbon atoms, between 120 and 10,000 carbon atoms, between 120 and 7000 carbon atoms, between 200 and 7000 carbon atoms, between 500 and 7000 carbon atoms, between 500 and 6500 carbon atoms, between 500 and 6000 carbon atoms, between 500 and 5500 carbon atoms, between 500 and 5000 carbon atoms, between 500 and 4500 carbon atoms, between 500 and 4000 carbon atoms, between 500 and 3500 carbon atoms, or between 3500 and 7000 carbon atoms.

c. Density

Optionally, the waste material contains a hydrocarbon polymer of ultra-low density, very low density, linear low or low density, linear medium or medium density, or high density or a combination thereof. For example, the waste material contains a hydrocarbon polymer with a mixture of low and high densities. Generally, a hydrocarbon polymer of ultra-low density is in the range from 0.867 g cm⁻³ to 0.889 g cm⁻³. A hydrocarbon polymer of very low density is typically in the range from 0.890 g cm⁻³ to 0.914 g cm⁻³. A hydrocarbon polymer of linear low or low density is typically in the range from 0.919 g cm⁻³ to 0.925 g cm⁻³. A hydrocarbon polymer of linear medium or medium density is typically in the range from 0.926 g cm⁻³ to 0.940 g cm⁻³. A hydrocarbon polymer of high density is typically in the range from 0.941 g cm⁻³ to 0.970 g cm⁻³. For example, a polyethylene of ultra-low density is typically in the range from 0.867 g cm⁻³ to 0.889 g cm⁻³; a polyethylene of very low density is typically in the range from 0.890 g cm⁻³ to 0.914 g cm⁻³; a polyethylene of linear low or low density is typically in the range from 0.919 g cm⁻³ to 0.925 g cm⁻³; a polyethylene of linear medium or medium density is typically in the range from 0.926 g cm⁻³ to 0.940 g cm⁻³; and a polyethylene of high density is in typically the range from 0.941 g cm⁻³ to 0.970 g cm⁻³.

For example, the waste material contains a linear low-density or low-density hydrocarbon polymer, such as a linear low-density or low-density polyethylene having a weight average molecular weight (Mw) in a range from 8.9×10⁴ g mol⁻¹ to 4.7×10⁵ g mol⁻¹, such as 94.5×10³ g mol⁻¹ and a density in a range from 0.919 g cm⁻³ to 0.925 g cm⁻³. Exemplary waste material that contains a low-density polyethylene includes freezer bags.

For example, the waste material contains a high-density hydrocarbon polymer, such as a high-density polyethylene having a Mw in a range from 10³ g mol⁻¹ to 10⁷ g mol⁻¹, such as 53.5×10³ g mol⁻¹ and a density in a range from 0.941 g cm⁻³ to 0.970 g cm⁻³. Exemplary waste material that contains a high-density polyethylene includes plastic bottles and plastic packaging, such as a plastic non-biodegradable grocery bags.

Optionally, the waste material contains a hydrocarbon polymer of different densities. For example, the waste material can contain a mixture of high density polyethylene and low density polyethylene.

d. Dispersity

Typically, the waste material contains hydrocarbon polymer having a high dispersity of >1.5, at least 2, at least 2.2, at least 2.5, at least 3, at least 3.5, up to 7.5, up to 7, between 1.8 and 7.5, between 2 and 7.5, or between 3.5 and 7.5, such as at least 1.9, at least 3.6, or at least 7.4.

2. Catalysts

The reactor contains a catalyst therein. The catalyst is generally in the form of a solid. Catalysts suitable for upcycling the waste material generally include a transition metal, optionally more than one transition metal. The term “transition metal” refers to a single transition metal or a transition metal that is an element in a compound, such as metal oxide or metal carbide. For example, the catalyst is a transition metal, a mixture of two or more metals containing at least one transition metal, a metal oxide of a transition metal, or a metal carbide of a transition metal, or a combination thereof. The transition metal of the catalyst acts as the catalytic active sites that react with the hydrocarbon polymer of the waste material.

Optionally, the hydrocarbon polymer and transition metal of the catalyst has a ratio (by mass) in a range from 20 to 200, from 30 to 200, from 30 to 150, from 35 to 200, or from 35 to 150, such as about 39 or about 133.

a. Transition Metals

Optionally, the catalyst is or contains a transition metal, such as platinum, palladium, ruthenium, iridium, rhenium, rhodium, iron, cobalt, nickel, copper, molybdenum, or tungsten.

Optionally, the catalyst is or contains a mixture of two or more metals containing a transition metal, such as a bimetallic or a trimetallic. For example, the catalyst is a mixture of two or more metals and each metal in the mixture of metals is a transition metal, such as platinum, palladium, ruthenium, iridium, rhodium, iron, cobalt, nickel, copper, molybdenum, or tungsten. For example, the catalyst is a bimetallic of platinum and cobalt.

For example, the catalyst is or contains a mixture of two or more metals containing a first group of transition metal(s) and a second group of metal(s). The first group of transition metals in the mixture of metals contains one or more transition metals, and each of the first group metals is platinum, palladium, ruthenium, iridium, rhodium, iron, cobalt, nickel, copper, molybdenum, or tungsten. The second group of metal(s) in the mixture of metals contains one or more metals that are different from the metals of the first group, and each of the second group metals can be a transition metal or a non-transition metal. For example, each of the second group metals is different from each of the transition metals in the first group and is rhenium, tin, lead, tungsten, molybdenum, chromium, manganese, or zinc. For example, the mixture of metals is a bimetallic containing platinum and rhenium or platinum and tin. For example, the mixture of meals is a trimetallic, and optionally contains platinum, rhenium, and tin.

b. Metal Oxides

Optionally, the catalyst is or contains a metal oxide of a transition metal. The metal oxide can contain a single transition metal or a mixture of two or more metals where at least one of the mixture of metals is a transition metal. For example, the catalyst is a metal oxide of a single transition metal, such as platinum, palladium, ruthenium, iridium, rhodium, iron, cobalt, nickel, copper, molybdenum, or tungsten.

For example, the catalyst is or contains a metal oxide of a mixture of two or more metals and each metal in the mixture of metals is a transition metal, such as platinum, palladium, ruthenium, iridium, rhodium, iron, cobalt, nickel, copper, molybdenum, or tungsten.

For example, the catalyst is or contains a metal oxide of a mixture of two or more metals containing a first group of transition metal(s) and a second group of metal(s) as described above.

Optionally, the catalyst is or contains a metal oxide having a perovskite structure. For example, the catalyst is a metal oxide of a transition metal, such as titanium, niobium, iron, or zirconium, and the catalyst further contains calcium and/or sodium, where the elements of the metal oxide are arranged in a way such that it has the same type of crystal structure as CaTiO₃.

c. Metal Carbides

Optionally, the catalyst is or contains a metal carbide of a transition metal. The metal carbide can contain a single transition metal or a mixture of two or more metals where at least one of the mixture of metals is a transition metal. For example, the catalyst is a metal carbide of a single transition metal, such as platinum, palladium, ruthenium, iridium, rhodium, iron, cobalt, nickel, copper, molybdenum, or tungsten. For example, the metal carbide is molybdenum carbide or tungsten carbide.

For example, the catalyst is or contains a metal carbide of a mixture of two or more metals and each metal in the mixture of metals is a transition metal, such as platinum, palladium, ruthenium, iridium, rhodium, iron, cobalt, nickel, copper, molybdenum, or tungsten.

For example, the catalyst is a metal carbide of a mixture of two or more metals containing a first group of transition metal(s) and a second group of metal(s) described above.

Optionally, more than one catalyst is used in the process. Any catalysts described above may be used. For example, the reactor contains more than one metal oxide described above therein. Each of the metal oxides in the reactor contains a transition metal that is different from the other. For example, the reactor contains more than one metal carbide described above therein. Each of the metal carbides in the reactor contains a transition metal that is different from the other. For example, the reactor contains a metal oxide and a metal carbide therein. The metal oxide in the reactor contains a transition metal that is different from the metal carbide.

d. Form of the Catalyst

The catalyst can be in a variety of suitable forms, such as atoms, nanoclusters, nanoparticles, or a combination thereof.

When the catalyst is in the form of nanoclusters, the nanoclusters can have an average diameter of up to 2 nm, up to 1.5 nm, up to 1 nm, in a range from 0.5 nm to 2 nm, from 0.5 nm to 1.5 nm, from 0.5 nm to 1 nm, or from 1 nm to 2 nm.

When the catalyst is in the form of nanoparticles, the nanoparticles can have an average diameter of up to 50 nm, up to 20 nm, up to 10 nm, up to 5 nm, at least 2 nm, in a range from 2 nm to 50 nm, from 2 nm to 45 nm, from 2 nm to 40 nm, from 2 nm to 35 nm, from 2 nm to 30 nm, from 2 nm to 25 nm, from 2 nm to 20 nm, from 2 nm to 15 nm, from 2 nm to 10 nm, or from 2 nm to 5 nm.

3. Optional Components

Optionally, the catalyst is dispersed on the surface of a support to form a catalytic system. When a support is present, the catalyst is dispersed on the surface of the support in the form of atoms, nanoclusters, or nanoparticles, or a combination thereof. For example, the catalyst is platinum nanoparticles and the platinum nanoparticles are dispersed on the surface of an aluminum oxide support. The support is generally understood to separate the catalyst atoms, nanoclusters, and/or nanoparticles apart and/or adsorb the hydrocarbon polymer(s), thereby improve the efficiency of the depolymerization reaction.

Additionally, some supports, such as acidic support materials or acid-modified support materials, can be included in the catalytic system to increase the efficiency of the catalyst therein. For example, a catalytic reaction using a catalytic system that includes an acidic support produces a liquid and/or wax product with a similar or higher yield and a similar or higher total selectivity to alkylaromatic in a shorter time period, compared to the same catalytic reaction using a catalytic system that does not include the acidic support, under the same reaction conditions. The term “same reaction conditions” means that the catalytic reaction is performed under the same temperature, same pressure, same atmosphere, etc. For example, depolymerization of polyethylene using a catalytic system that includes an acidic support produces a liquid and/or wax product with a similar or higher yield with a similar or higher total selectivity to alkylaromatic in a time period that is at least 2-time shorter, at least 3-time shorter, at least 5-time shorter, at least 10-time shorter, at least 15-time shorter, or at least 20-time shorter, compared to the same catalytic reaction using a catalytic system that does not include the acidic support, under the same reaction conditions. Optionally, these support materials are catalytic on their own.

Optionally, more than one support is used for forming a catalytic system with the catalyst. When two or more supports are present, the catalyst is dispersed on the surface of at least one of the supports in the form of atoms, nanoclusters, or nanoparticles, or a combination thereof. For example, when two supports are used for forming a catalytic system, the catalyst is platinum nanoparticles and the platinum nanoparticles are dispersed on the surface of a first support; the platinum nanoparticles dispersed first support is mixed with a second support that is different from the first support to form the catalytic system. For example, when two supports are used for forming a catalytic system, the catalyst is platinum nanoparticles and the platinum nanoparticles are dispersed on the surface of a first support and the surface of a second support that is different from the first support; the platinum nanoparticles dispersed first support and second support are mixed to form the catalytic system.

Optionally, when two or more supports are present, a first catalyst can be dispersed on the surface of at least one of the supports in the form of atoms, nanoclusters, or nanoparticles, or a combination thereof, and a second catalyst can be dispersed on the surface of at least one of the supports that is different from the support having the first catalyst dispersed thereon, in the form of atoms, nanoclusters, or nanoparticles, or a combination thereof. For example, when two supports are used for forming a catalytic system, a first catalyst is platinum nanoparticles and the platinum nanoparticles are dispersed on the surface of a first support and a second catalyst that is different from the first catalyst is dispersed on the surface of a second support that is different from the first support.

When two or more supports are present, the weight percentage of each support in the catalytic system is selected based on the specific catalyst and the material of each support. For example, when two supports are used for forming a catalytic system, a first support having platinum nanoparticles dispersed thereon is mixed with a second support without any catalyst dispersed therein, where the second support is different from the first support. The weight percentage of the second support in the catalytic system is in a range from 2% to 90%, from 2% to 50%, from 2% to 35%, from 10% to 90%, from 10% to 80%, from 10% to 70%, from 10% to 60%, from 10% to 50%, from 10% to 30%, from 15% to 40%, from 20% to 50%, from 20% to 40%, or from 20% to 35%, such as about 20% or about 33%, with the balance of the components in the catalytic system being the weight of first support and the weight of the catalyst dispersed thereon.

a. Materials for Support

The material for the support is typically chemically stable when subject to the operating conditions of the reactor for the depolymerization of the waste material. For example, the material for the support does not react with common chemicals (e.g. hydrogen gas, saturated C1-C5 alkane, benzene, or toluene, etc) at the reaction temperature and pressure and for a period of time described below. For example, the material for the support does not react with common chemicals (e.g. hydrogen gas, saturated C1-C5 alkane, benzene, or toluene, etc) at a temperature of 500° C. or less and at a pressure in a range from 1 atm to 2 atm.

Examples of suitable materials for the support include, but are not limited to, oxide compounds, halogenated oxide compounds, metal carbides, metal phosphates, and carbon-based materials, and acid modified oxides, metal carbides, metal phosphates, and carbon-based materials. The material for the support is a different material than the transition metal or mixture of metals in the catalyst.

Optionally, the material for the support is an oxide compound, such as a metal oxide or a non-metal oxide. For example, the oxide compound for the support is silicon dioxide, aluminum oxide (e.g. γ-alumina, amorphous alumina, etc.), halogenated alumina, such as Cl-alumina and F-alumina, silica-aluminum oxide, silica (e.g. silica gel, mesoporous silica, etc.), acid-modified silica, CeO₂, acid-modified CeO₂, TiO₂, acid-modified TiO₂, WO₃, MoO₃, Re₂O₇, a perovskite, clays (dealuminated clays, clays modified with organic or inorganic acids, clays ion exchanged with H⁺, NH4⁺, Fe³⁺, Na⁺, etc.), or a zeolite (e.g. ZSM-5, dealuminated zeolites, zeolites modified by organic and/or inorganic acids, zeolites modified by organic and/or inorganic bases, etc.), or a combination thereof.

Examples of acids that are suitable for modifying the above-mentioned support materials include, but are not limited to, Keggin-type heteropoly acid (H₃PW₁₂O₄₀, H₄SiW₁₂O₄₀, etc.) and their acidic salt thereof (e.g. Cs_{2.5}H_{0.5}PW₁₂O₄₀, etc.). Methods for modifying the support materials are known in the art, such as chemical modifications (e.g. covalently attaching an acid/base on the material) or non-chemical modifications (e.g. physical loading of an acid/base on or entrapping an acid/base in the material). For example, Keggin-type heteropoly acid (H₃PW₁₂O₄₀, H₄SiW₁₂O₄₀, etc.) or an acidic salt thereof (e.g. Cs_{2.5}H_{0.5}PW₁₂O₄₀, etc.) can be physically loaded on a silica support, a TiO₂ support, or a carbon-based material.

Optionally, the material for the support is a metal carbide. For example, the metal carbide forming the support is any metal carbide described above, such as molybdenum carbide or tungsten carbide.

Optionally, the material for the support is a metal phosphate. The metal phosphate forming the support can contain a transition metal or a mixture of metals described above for metal oxides.

Optionally, the material for the support is a carbon-based material. A carbon-based material generally refers to a material where the number of carbon atoms are at least 50% of the total number of atoms in the material. Examples of suitable carbon-based materials for the support include, but are not limited to, graphite, graphite oxide, activated carbon, carbon nanotubes, carbon nanosheet, graphene, and graphene oxide, and an acid modified version thereof, or a combination thereof. Suitable acids for modifying these carbon-based materials can be any of the inorganic acids described above, such as Keggin-type heteropoly acid (H₃PW₁₂O₄₀, H₄SiW₁₂O₄₀, etc.) and their acidic salt thereof (e.g. Cs_{2.5}H_{0.5}PW₁₂O₄₀, etc.).

Optionally, the material for the support is a Keggin-type heteropoly acid (H₃PW₁₂O₄₀, H₄SiW₁₂O₄₀, etc.) or an acidic salt of the Keggin-type heteropoly acid (Cs_{2.5}H_{0.5}PW₁₂O₄₀, etc.).

The material for the support may be catalytic or non-catalytic. For example, the support is formed from a catalytic material that can participate in the depolymerization reaction. In these cases, the catalytic material for the support may be a transition metal, a mixture of metals containing at least one transition metal, a metal oxide containing a transition metal, or a metal carbide containing a transition metal as described above as suitable materials for the catalyst. However, the catalytic material for the support is different from the metal in the catalyst. Optionally, the material for the support is a material that does not participate in the depolymerization reaction, such as silica oxide or a carbon-based material. For example, the material for the support is γ-alumina or silica-alumina.

Optionally, the material for the support is an acidic material. Typically, the acidic material is strong enough to protonate pyridine, as determined by known methods. Whether a given material is able to protonate pyridine, and therefore considered to be an acidic material can be shown using the following test. First the, material is dehydrated at about 400° C. under vacuum; then the dehydrated material is dosed with an excess of pyridine vapor at room temperature, followed by desorption, typically at about 150° C. under vacuum for suitable time period, such as about 20 minutes. If the IR spectra of the resulting material shows a peak at 1545 cm⁻¹, strong Brønsted acid sites are present, and thus demonstrates its acidity for protonating pyridine. Examples of suitable acidic materials that can be used for the support include, but are not limited to, halogenated oxide compounds (e.g. Cl-alumina and F-alumina), WO₃, MoO₃, Re₂O₇, acid modified oxide compounds, such as acid-modified zeolites, silica, CeO₂, TiO₂, clays, etc., and acid modified carbon-based materials (e.g. acid-modified activated carbon, carbon nanotubes, graphene, etc.), where the acid can be any of the acids described above. When the material for the support is a halogenated oxide compound, such as Cl-alumina or F-alumina, the weight percentage of the halogen in the support can be in a range from 0.1% to 5%, from 0.5% to 3%, from 0.5% to 2%, from 0.5% to 1.5%, such as about 0.7% or about 1.3%.

Optionally, when a single support is used for forming a catalytic system, such as any one of the oxide compounds, halogenated oxide compounds, metal carbides, metal phosphates, and carbon-based materials, and their acid modified versions thereof described above, the weight percentage of the support in the catalytic system is in a range from 90% to 99.9%, from 90% to 99.8%, from 90% to 99.5%, from 90% to 99%, from 98% to 99.9%, from 98% to 99.8%, from 98% to 99.5%, from 98% to 99%, from 98.5% to 99.9%, from 98.5% to 99.8%, from 98.5% to 99.5%, or from 99% to 99.9%, with the balance of the components in the catalytic system being the weight of the catalyst dispersed thereon.

Optionally, when two or more supports are used for forming a catalytic system, at least one of the supports is an acidic support as described above, such as a halogenated oxide compound (e.g. Cl-alumina and F-alumina). In these catalytic systems, the catalyst can be dispersed on the surface of the acidic support(s) and/or on the surface of the support material that is different from the acidic support(s).

For example, when two supports are used for forming a catalytic system, a first support is γ-alumina or silica-alumina having the catalyst dispersed thereon and a second support is an acidic support as described above, such as F-alumina, that does not have the catalyst dispersed thereon. The weight percentage of the acidic support in the catalyst system is in a range from 2% to 90%, from 2% to 50%, from 2% to 35%, from 10% to 90%, from 10% to 80%, from 10% to 70%, from 10% to 60%, from 10% to 50%, from 10% to 30%, from 15% to 40%, from 20% to 50%, from 20% to 40%, or from 20% to 35%, such as about 20% or about 33%, with the balance of the components in the catalytic system being the weight of the first support and the weight of the catalyst dispersed thereon.

For example, when two supports are used for forming a catalytic system, a first support is γ-alumina or silica alumina having the catalyst dispersed thereon and a second support is an acidic support as described above, such as F-alumina, that also has the catalyst dispersed thereon. The weight percentage of the acidic support in the catalyst system is in a range from 2% to 90%, from 2% to 50%, from 2% to 35%, from 10% to 90%, from 10% to 80%, from 10% to 70%, from 10% to 60%, from 10% to 50%, from 10% to 30%, from 15% to 40%, from 20% to 50%, from 20% to 40%, or from 20% to 35%, such as about 20% or about 33%, with the balance of the components in the catalytic system being the weight of the first support and the weight of the catalyst dispersed on the first and second supports. The catalyst dispersed on the first support can be the same as or different from the catalyst that is dispersed on the second support.

b. Forms of the Support

Typically, the support has a surface area having a dimension sufficiently large to allow the catalyst to be dispersed hereon. When calculating surface area of the support, any surface that the reactants are able to contact is typically included. Surface area of the support can be measured by techniques known in the art, for example, by nitrogen physisorption. Typically, the support has a surface area of at least 50 m² g⁻¹, at least 100 m² g⁻¹, or at least 150 m² g⁻¹, in a range from 50 m² g⁻¹ to 1000 m² g⁻¹, from 50 m² g⁻¹ to 900 m² g⁻¹, from 50 m² g¹ to 800 m² g⁻¹, from 50 m² g¹ to 700 m² g¹, from 50 m² g⁻¹ to 600 m² g⁻¹, from 50 m² g¹ to 500 m² g⁻¹, from 50 m² g⁻¹ to 400 m² g⁻¹, from 50 m² g¹ to 300 m² g⁻¹, from 50 m² g¹ to 200 m² g⁻¹, or from 50 m² g⁻¹ to 1000 m² g⁻¹. For example, the support has a surface area of at least 150 m² g⁻¹, such as 186 m² g⁻¹.

The support can be in a form of a mesoporous sheet or microparticles, or a combination thereof. For example, the support can be in the form of aluminum oxide microparticles. In some embodiments, the support can be in the form of a carbon sheet (e.g. graphene sheet), carbon powders, carbon particles, or carbon nanoparticles (e.g. carbon nanotubes).

The support is generally understood to separate the catalyst atoms, nanoclusters, and/or nanoparticles apart and/or adsorb the hydrocarbon polymer(s), thereby improve the efficiency of the depolymerization reaction. For example, when the catalyst is a metal or a mixture of metals dispersed on the support in the form of atoms, each atom is displaced a distance or different distances from the other atoms of the metal or mixture of metals. For example, when the catalyst is dispersed on the support in the form of nanoclusters, each nanocluster is displaced a distance or different distances from the other nanoclusters of the catalyst. For example, when the catalyst is dispersed on the support in the form of nanoparticles, each nanoparticle is displaced a distance or different distances from the other nanoparticles of the catalyst.

c. Weight Loading

Generally, the total weight loading of the one or more metals of the catalyst is present on the support in an amount less than 10 wt % of the total weight of the catalyst and the support. For example, when the catalyst includes one or more transition metals, the total weight loading of the metals present in the catalyst on the support is calculated as the sum of the weights of the transition metals in the catalyst divided by the sum of the weight of the support plus the weights of the transition metals and non-transition metals multiplied by 100. When the catalyst includes only one transition metal, the total weight loading of the metals present in the catalyst on the support is simply calculated as the weight of the transition metal in the catalyst divided by the sum of the weight of the support plus the weight of the transition metal multiplied by 100. Similarly, when the catalyst includes both one or more transition metals and one or more non-transition metals, the total weight loading of the metals present in the catalyst on the support is calculated as the sum of the weights of transition metals and non-transition metals in the catalyst divided by the sum of the weight of the support plus the weights of the transition metals and non-transition metals multiplied by 100.

For example, the total weight loading of the one or more metals of the catalyst is present on the support in an amount of less than 10 wt %, less than 8 wt %, less than 5 wt %, less than 4.5 wt %, less than 4 wt %, less than 3.5 wt %, less than 3 wt %, less than 2.5 wt %, less than 2 wt %, less than 1.5 wt %, less than 1 wt %, in a range of from 0.1 wt % to 10 wt %, from 0.1 wt % to 8 wt %, from 0.1 wt % to 5 wt %, from 0.2 wt % to 5 wt %, from 0.5 wt % to 10 wt %, from 0.5 wt % to 8 wt %, from 0.5 wt % to 5 wt %, from 1 wt % to 10 wt %, from 1 wt % to 8 wt %, from 1 wt % to 5 wt %, from 0.1 wt % to 4.5 wt %, from 0.1 wt % to 4 wt %, from 0.1 wt % to 3.5 wt %, from 0.1 wt % to 3 wt %, from 0.1 wt % to 2.5 wt %, or from 0.1 wt % to 2 wt %, from 0.1 wt % to 1.5 wt %, from 0.1 wt % to 1 wt %, or from 0.1 wt % to 0.5 wt % of the total weight of the catalyst and the support, such as about 0.2 wt %, about 1 wt %, or about 1.5 wt % of the total weight of the catalyst and the support.

Methods for preparing the disclosed catalysts are known. For example, the disclosed catalysts may be prepared using the methods described in Garcia and Goto, Mater. Trans., 44(9):1717-1728 (2003).

B. Operate the Reactor to Convert the Hydrocarbon Polymer to a Product.

The reactor is operated at a sufficient temperature for a sufficient period of time to convert the hydrocarbon polymer to a product containing an alkylaromatic compound, optionally more than one alkylaromatic compound. The product is in the form of a liquid, a wax, or a combination thereof. Typically, the mol % of the alkylaromatic compound in the product, optionally the total mol % of the more than one alkylaromatic compound in the product is at least 50 mol %.

Typically, the reactor is operated at a low temperature, such as less than or equal to 500° C., optionally less than or equal to 450° C., less than or equal to 400° C., or less than or equal to 350° C. The temperature at which the reactor operates is sufficient to convert the hydrocarbon polymer to the product containing one or more alkylaromatic compounds, such as dialkylbenzene.

An exemplary upcycling process, which includes the steps of (i) feeding a waste material containing hydrocarbon polymer(s) into a reactor; and (ii) operating the reactor at a sufficient temperature for a sufficient period of time to convert the hydrocarbon polymer to a product, is schematically illustrated in FIG. 14. As shown in FIG. 14, the process for upcycling a waste material typically includes feeding the plastic waste material containing one or more hydrocarbon polymer(s) into a reactor 10 via waste material stream 100. The reactor 10 contains a catalyst therein. The catalyst is fed into the reactor via the catalyst stream 200. Optionally a reagent/solvent is fed into the reactor 10 via reagent stream 210. The catalyst and optionally the reagent/solvent can be fed into the reactor 10 prior to, simultaneously, or subsequent to feeding the waste material into the reactor 10. Following step (i), the waste material is in contact with the catalyst in the reactor 10. In step (ii), the reactor 10 is heated via heating path 220 to a sufficient temperature and maintained at this temperature for a sufficient period of time to convert the hydrocarbon polymer to the product stream 110 in the form of a liquid and/or wax.

The product stream 110 contains a liquid and/or wax containing an alkylaromatic compound or more than one alkylaromatic compound. The mol % of the alkylaromatic compound or the total mol % of the more than one alkylaromatic compound in the product stream 110 is at least 50 mol %. The product stream 110 contains other unsaturated compounds and/or C₆-C₁₀ alkanes in addition to the one or more alkylaromatic compound(s). In addition to the liquid/wax stream, gas products also exit the reactor. Additionally, some unreacted solid waste may remain. The solid waste can be removed from the reactor 10 via the organic solid stream 130. The gas product containing hydrogen gas and/or a short-chain hydrocarbon(s), such as a saturated C₁-C₅ alkane, benzene, or toluene, or a combination thereof exits the reactor via gas stream 120. Optionally, the product stream 110 is fed to a separation unit 20 where the product is separated into short-chain (i.e. C₆-C₁₀) alkane and/or aromatic compound stream 111, alkane stream 112 containing a mixture of alkanes with carbon numbers in a range from 11 to 80, thus the alkane stream contains alkanes with an average carbon number of 30, and/or alkylaromatic compound stream 113 containing alkylaromatic compound(s) with average carbon numbers described below, such as alkylaromatic compound(s) with an average carbon number of 30.

1. Product

Following steps (i) and (ii), product in the form of a liquid, a wax, or a combination thereof, is formed. The product in a liquid and/or wax form is identified as stream 110 in FIG. 14. Optionally, the wax can become a flowable liquid when heated to a temperature of at least 45° C.

The product contains an alkylaromatic compound, optionally more than one alkylaromatic compound. For example, the product contains a mixture of two alkylaromatic compounds, a mixture of three alkylaromatic compounds, a mixture of four alkylaromatic compounds, a mixture of five alkylaromatic compounds, and optionally contains more than five different alkylaromatic compounds. Optionally, the product contains other unsaturated compounds, alkanes with a carbon number in a range from 11 to 80, from 15 to 80, from 20 to 80, from 11 to 70, from 11 to 60, from 11 to 50, from 20 to 50, such as a mixture of alkanes with carbon numbers in a range from 11 to 80, thus the alkane stream contains alkanes with an average carbon number of 30, and/or C₆-C₁₀ alkanes in addition to the alkylaromatic compound(s). Optionally, the product does not include tetramethylbenzene, butylbenzene, dimethylnaphthalene, or biphenyl, or a combination thereof.

a. Aromatic Compounds

The liquid and/or wax product contains an alkylaromatic compound, and optionally contains more than one alkylaromatic compound. For example, the product contains a mixture of two alkylaromatic compounds, three alkylaromatic compounds, four alkylaromatic compounds, or five alkylaromatic compounds, and optionally contains more than five different alkylaromatic compounds. The alkylaromatic compound or each alkylaromatic compound in the mixture of alkylaromatic compounds can be monocyclic or polycyclic alkylaromatic hydrocarbons, or partially hydrogenated analogs of polycyclic alkylaromatic hydrocarbons. The alkylaromatic compounds can be monocyclic aromatic hydrocarbons or polycyclic aromatic hydrocarbons, or partially hydrogenated analogs of polycyclic aromatic hydrocarbons. In polycyclic aromatic hydrocarbons, the rings can be attached together in a pendant manner or can be fused. After exiting the reactor, the alkylaromatic compounds can be separated from the other compounds in the liquid and/or wax product stream in a separation unit. For example, in the exemplary process shown in FIG. 14, the separation unit 20 separates the compounds in the liquid/wax product stream 110 to produce an alkylaromatic compound(s) stream 113.

Exemplary alkylaromatic compounds that can be in the liquid and/or wax product include alkylbenzene, an alkyl-polycyclic aromatic compound (e.g. alkylnaphthalene, alkylanthracene, alkylphenanthrene) or a partially hydrogenated analog of an alkyl-polycyclic aromatic compound (e.g. a partially hydrogenated analog of alkylnaphthalene, alkylanthracene, or alkylphenanthrene), such as a partially hydrogenated analog of alkylnaphthalene (i.e. alkyltetralin), or a combination thereof. For example, the product contains dialkylbenzene and/or dialkylnaphthalene, and optionally alkyltetralin, alkylanthracene, and/or alkylphenanthrene.

Optionally, the liquid and/or wax product does not include tetramethylbenzene, butylbenzene, dimethylnaphthalene, or biphenyl.

i. Number of Carbons

Typically, each alkylaromatic compound in the liquid and/or wax product contains at least 10 carbon atoms, at least 12 carbon atoms, up to 50 carbon atoms, up to 45 carbon atoms, up to 40 carbon atoms, up to 35 carbon atoms, up to 30 carbon atoms, up to 25 carbon atoms, in a range of from 10 to 50 carbon atoms, from 10 to 45 carbon atoms, from 10 to 40 carbon atoms, from 10 to 35 carbon atoms, from 10 to 30 carbon atoms, from 10 to 25 carbon atoms, from 12 to 50 carbon atoms, from 12 to 45 carbon atoms, from 12 to 40 carbon atoms, from 12 to 35 carbon atoms, from 12 to 30 carbon atoms, from 12 to 25 carbon atoms, from 15 to 50 carbon atoms, from 15 to 45 carbon atoms, from 15 to 40 carbon atoms, from 15 to 35 carbon atoms, from 15 to 30 carbon atoms, from 15 to 25 carbon atoms, from 20 to 50 carbon atoms, from 20 to 45 carbon atoms, from 20 to 40 carbon atoms, from 20 to 35 carbon atoms, from 20 to 30 carbon atoms, or from 20 to 35 carbon atoms.

Optionally, the alkylaromatic compound in the liquid and/or wax product contains from 10 to 50 carbon atoms, from 12 to 50 carbon atoms, from 15 to 50 carbon atoms, 15 to 25 carbons, 20 to 40 carbons, or from 20 to 35 carbon atoms.

For example, the alkylaromatic compound in the liquid and/or wax product contains from 10 to 50 carbon atoms, from 12 to 50 carbons, from 15 to 50 carbon atoms, 15 to 25 carbons, 20 to 40 carbons, or from 20 to 35 carbon atoms. For example, the aromatic compound in the product is a C₁₀-C₅₀ alkylbenzene, a polycyclic C₁₀-C₅₀ aromatic, or a C₁₀-C₅₀ alkyl-polycyclic aromatic, optionally a C₁₅-C₅₀ alkylbenzene, a polycyclic C₁₅-C₅₀ aromatic, or a C₁₅-C₅₀ alkyl-polycyclic aromatic, optionally a C₁₅-C₂₅ alkylbenzene, a polycyclic C₁₅-C₂₅ aromatic, or a C₁₅-C₂₅ alkyl-polycyclic aromatic, optionally a C₂₀-C₄₀ alkylbenzene, a polycyclic C₂₀-C₄₀ aromatic, or a C₂₀-C₄₀ alkyl-polycyclic aromatic, optionally a C₂₀-C₃₅ alkylbenzene, a polycyclic C₂₀-C₃₅ aromatic, or a C₂₀-C₃₅ alkyl-polycyclic aromatic.

Optionally, the liquid and/or wax product does not include tetramethylbenzene, butylbenzene, dimethylnaphthalene, or biphenyl or a combination thereof.

ii. Average Molecular Weight

Typically, the alkylaromatic compound in the liquid and/or wax product has a Mw less than 800 g mol⁻¹, less than 750 g mol⁻¹, less than 700 g mol⁻¹, less than 650 g mol⁻¹, less than 600 g mol⁻¹, less than 550 g mol⁻¹, less than 500 g mol⁻¹, at least 300 g mol⁻¹, at least 200 g mol⁻¹, at least 150 g mol⁻¹, in a range of from 150 g mol⁻¹ to 800 g mol⁻¹, from 200 g mol⁻¹ to 800 g mol⁻¹, from 200 g mol⁻¹ to 600 g mol⁻¹, from 200 g mol⁻¹ to 500 g mol⁻¹, from 300 g mol⁻¹ to 800 g mol⁻¹, from 300 g mol⁻¹ to 600 g mol⁻¹, or from 300 g mol⁻¹ to 500 g mol⁻¹.

Optionally, the alkylaromatic compound in the liquid and/or wax product has a Mn less than 700 g mol⁻¹, less than 650 g mol⁻¹, less than 600 g mol⁻¹, less than 550 g mol⁻¹, less than 500 g mol⁻¹, at least 300 g mol⁻¹, at least 200 g mol⁻¹, at least 150 g mol⁻¹, in a range of from 150 g mol⁻¹ to 700 g mol⁻¹, from 200 g mol⁻¹ to 700 g mol⁻¹, from 200 g mol⁻¹ to 600 g mol⁻¹, from 200 g mol⁻¹ to 500 g mol⁻¹, from 300 g mol⁻¹ to 700 g mol⁻¹, from 300 g mol⁻¹ to 600 g mol⁻¹, or from 300 g mol⁻¹ to 500 g mol⁻¹.

For example, the alkylaromatic compound in the liquid and/or wax product has a Mw less than 800 g mol⁻¹, less than 750 g mol⁻¹, less than 700 g mol⁻¹, less than 650 g mol⁻¹, less than 600 g mol⁻¹, less than 550 g mol⁻¹, less than 500 g mol⁻¹, at least 300 g mol⁻¹, at least 200 g mol⁻¹, at least 150 g mol⁻¹, in a range of from 150 g mol⁻¹ to 800 g mol⁻¹, from 200 g mol⁻¹ to 800 g mol⁻¹, from 200 g mol⁻¹ to 600 g mol⁻¹, from 200 g mol⁻¹ to 500 g mol⁻¹, from 300 g mol⁻¹ to 800 g mol⁻¹, from 300 g mol⁻¹ to 600 g mol⁻¹, or from 300 g mol⁻¹ to 500 g mol⁻¹.

Optionally, the alkylaromatic compound in the liquid and/or wax product has a Mw or Mn that is at least 5-times less than the Mw or Mn of the hydrocarbon polymer (reactant) in the waste material before depolymerization, at least 10-times less than the Mw or Mn of the hydrocarbon polymer, at least 15-times less than the Mw or Mn of the hydrocarbon polymer, at least 20-times less than the Mw or Mn of the hydrocarbon polymer, at least 25-times less than the Mw or Mn of the hydrocarbon polymer, at least 30-times less than the Mw or Mn of the hydrocarbon polymer, at least 35-times less than the Mw or Mn of the hydrocarbon polymer, at least 40-times less than the Mw or Mn of the hydrocarbon polymer, at least 45-times less than the Mw or Mn of the hydrocarbon polymer, at least 50-times less than the Mw or Mn of the hydrocarbon polymer, at least 55-times less than the Mw or Mn of the hydrocarbon polymer, at least 60-times less than the Mw or Mn of the hydrocarbon polymer, at least 65-times less than the Mw or Mn of the hydrocarbon polymer, at least 70-times less than the Mw or Mn of the hydrocarbon polymer, at least 75-times less than the Mw or Mn of the hydrocarbon polymer, at least 80-times less than the Mw or Mn of the hydrocarbon polymer, at least 85-times less than the Mw or Mn of the hydrocarbon polymer, at least 90-times less than the Mw or Mn of the hydrocarbon polymer, at least 95-times less than the Mw or Mn of the hydrocarbon polymer, at least 100-times less than the Mw or Mn of the hydrocarbon polymer, at least 120-times less than the Mw or Mn of the hydrocarbon polymer, at least 150-times less than the Mw or Mn of the hydrocarbon polymer, or at least 200-times less than the Mw or Mn of the hydrocarbon polymer.

For example, the alkylaromatic compound in the liquid and/or wax product has a Mw at least 5-times less than the Mw of the hydrocarbon polymer (reactant) of the waste material before depolymerization, at least 10-times less than the Mw of the hydrocarbon polymer (reactant), at least 20-times less than the Mw of the hydrocarbon polymer (reactant), at least 50-times less than the Mw of the hydrocarbon polymer (reactant), at least 100-times less than the Mw of the hydrocarbon polymer (reactant), at least 150-times less than the Mw of the hydrocarbon polymer (reactant), or at least 200-times less than the Mw of the hydrocarbon polymer (reactant).

For example, the alkylaromatic compound in the liquid and/or wax product has a Mn at least 5-times less than the Mn of the hydrocarbon polymer (reactant) of the waste material before depolymerization, at least 10-times less than the Mn of the hydrocarbon polymer (reactant), at least 20-times less than the Mn of the hydrocarbon polymer (reactant), at least 50-times less than the Mn of the hydrocarbon polymer (reactant), at least 100-times less than the Mn of the hydrocarbon polymer (reactant), at least 150-times less than the Mn of the hydrocarbon polymer (reactant), or at least 200-times less than the Mn of the hydrocarbon polymer (reactant).

iii. Dispersity

Generally, the dispersity (polydispersity index, PDI) of the distribution of the molecular weight of the alkylaromatic compound in the liquid and/or wax product is smaller than the dispersity of the distribution of the molecular weight of the hydrocarbon polymer (reactant) prior to depolymerization.

Typically, the alkylaromatic compound in the liquid and/or wax product has a dispersity of less than 4.0, less than 3.5, less than 3.0, less than 2.5, less than 2.0, less than 1.5, less than 1.3, less than 1.2, between 1.1 and 1.5, between 1.1 and 1.4, between 1.1 and 1.3, or between 1.1 and 1.2.

iv. Structure of the Alkylaromatic Compounds

The alkylaromatic compound in the liquid and/or wax product can contain two alkyl groups, three alkyl groups, four alkyl groups, five alkyl groups, six alkyl groups, or more, attached to the aromatic ring or partially hydrogenated ring of a polycyclic aromatic compound.

Generally, the alkyl group of the alkylaromatic compound in the product can be linear, branched, or cyclic. The alkyl group of the alkylaromatic compound in the product can be a linear C₁-C₂₀ alkyl, a branched C₄-C₂₀ alkyl, or a cyclic C₃-C₂₀ alkyl, optionally, a linear C₁-C₁₅ alkyl, a branched C₄-C₁₅ alkyl, or a cyclic C₃-C₁₅ alkyl, optionally a linear C₁-C₁₂ alkyl, a branched C₄-C₁₂ alkyl, or a cyclic C₃-C₁₂ alkyl, optionally a linear C₁-C₁₀ alkyl, a branched C₄-C₁₀ alkyl, or a cyclic C₃-C₁₀ alkyl, optionally a linear C₁-C₅ alkyl, a branched C₄-C₅ alkyl, or a cyclic C₃-C₅ alkyl.

Optionally, the alkyl group of the alkylaromatic compound in the product is a linear C₁-C₂₀, C₁-C₁₅, C₁-C₁₂, C₁-C₁₀, C₁-C₈, C₁-C₅, C₂-C₂₀, C₂-C₁₅, C₂-C₁₂, C₂-C₁₀, C₂-C₈, C₂-C₅, C₃-C₂₀, C₃-C₁₅, C₃-C₁₂, C₃-C₁₀, C₃-C₈, C₃-C₅, C₄-C₂₀, C₄-C₁₅, C₄-C₁₂, C₄-C₁₀, C₄-C₈, C₄-C₅, C₅-C₂₀, C₅-C₁₅, C₅-C₁₂, C₅-C₁₀, or C₅-C₈ alkyl group.

Optionally, the alkylaromatic compound contains two alkyl groups attached to the aromatic ring of the alkylaromatic compound, or aromatic ring and/or partially hydrogenated ring in case of a polycyclic aromatic compound. The two alkyl groups can have any suitable relative positions. The second alkyl group may be at the ortho-, para-, or meta-position relative to the first alkyl group. The second alkyl group may be in the same ring or different rings in case of the polycyclic aromatic compound. For example, a biaryl compound contains two alkyl groups, where the first alkyl group is attached to a first aromatic ring and the second alkyl group is attached to a second aromatic ring. For example, an alkyltetralin contains two alkyl groups, where the first alkyl group is attached to a first aromatic ring and the second alkyl group is attached to a second partially hydrogenated ring.

Optionally, the alkylaromatic compound in the product is a dialkylbenzene. Optionally, the dialkylbenzene has a structure of Formula (I).

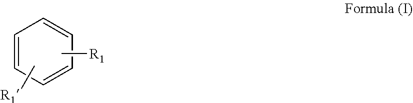

where R₁ and R₁′ are independently a C₁-C₂₀ alkyl group, a C1-C₁₅ alkyl group, a C1-C₁₂ alkyl group, a C₁-C₁₀ alkyl group, or a C₁-C₅ alkyl group, and where the sum of the carbon atoms in R₁ and R₁′ is at least 10.

Optionally, R₁ and R₁′ of Formula (I) are independently a linear C₁-C₂₀ alkyl group, a linear C₁-C₁₅ alkyl group, a linear C₁-C₁₂ alkyl group, a linear C₁-C₁₀ alkyl group, or a linear C₁-C₅ alkyl group, and where the sum of the carbon atoms in R₁ and R₁′ is at least 10.

Optionally, the alkylaromatic compound in the product is a polycyclic aromatic compound. The polycyclic aromatic compound can contain two aromatic rings, three aromatic rings, four aromatic rings, five aromatic rings, or six aromatic rings. For example, the alkylaromatic compound in the product is alkylnaphthalene, alkylanthracene, or alkylphenanthrene. Optionally, the alkylaromatic compound in the product is a partially hydrogenated analog of a polycyclic aromatic compound. For example, the alkylaromatic compound in the product is a partially hydrogenated analog of alkylnaphthalene, i.e. alkyltetralin.

Optionally, the polycyclic aromatic compound is a dialkylnaphthalene. Optionally, the dialkylnaphthalene has a structure of Formula (II).

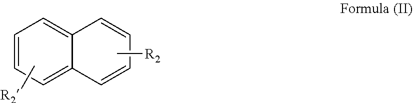

where R₂ and R₂′ are independently a C₁-C₂₀ alkyl group, a C₁-C₁₅ alkyl group, a C₁-C₁₂ alkyl group, a C₁-C₁₀ alkyl group, or a C₁-C₅ alkyl group, and where the sum of the carbon atoms in R₂ and R₂′ is at least 4, optionally at least 5.

Optionally, R₂ and R₂′ of Formula (II) are a linear C₁-C₂₀ alkyl group, a linear C₁-C₁₅ alkyl group, a linear C₁-C₁₂ alkyl group, a linear C₁-C₁₀ alkyl group, or a linear C₁-C₅ alkyl group, and where the sum of the carbon atoms in R₂ and R₂′ is at least 4, optionally at least 5.

b. Other Compounds

Optionally, the liquid and/or wax product further contains other unsaturated compounds, alkanes with a carbon number in a range from 11 to 80, from 15 to 80, from 20 to 80, from 11 to 70, from 11 to 60, from 11 to 50, from 20 to 50, such as a mixture of alkanes with carbon numbers in a range from 11 to 80, thus the alkane stream contains alkanes with an average carbon number of 30, and/or C₆-C₁₀ alkanes in addition to the alkylaromatic compound(s). The total mol % of the other compounds in the liquid and/or wax product is typically less than 50 mol %, such as less than 45 mol %, less than 40 mol %, less than 35 mol %, less than 30 mol %, less than 25 mol %, less than 20 mol %, less than 15 mol %, or less than 10 mol %.

For example, the liquid and/or wax product contains an aromatic compound that is not an alkylaromatic compound. For example, the product contains a polycyclic aromatic compound (e.g. naphthalene, anthracene, phenanthrene, or a partially hydrogenated analog of naphthalene, anthracene, or phenanthrene, such as tetralin) or an alkylaromatic compound (e.g. alkylbenzene, alkylnaphthalene, alkylanthracene, alkylphenanthrene, or a partially hydrogenated analog of alkylnaphthalene, alkylanthracene, or alkylphenanthrene, such as alkyltetralin), or a combination thereof.

For example, the liquid and/or wax product contains an olefin, such as a dienen or a cycloalkene. Olefins are unsaturated hydrocarbons that contain at least one carbon-carbon double bond. The olefin in the product can be linear, branched, or cyclic. For example, the unsaturated compound in the product is diene or a cycloalkene.

The other unsaturated compounds can contain any number of carbons described above for the alkylaromatic compound. The other unsaturated compounds can have a Mw or Mn as described above for the alkylaromtic compound.

The other unsaturated compounds can be separated from the alkylaromatic compounds in the liquid and/or wax product. For example, after exiting the reactor, the liquid and/or wax product stream can be fed into a separation unit. For example, as shown in FIG. 14, the separation unit 20 separates the liquid/wax product stream 110 to produce a C₆-C₁₀ alkane and aromatic compound stream 111 and an alkane stream 112 (i.e. a mixture of alkanes with carbon numbers in a range from 11 to 80, thus the alkane stream contains alkanes with an average carbon number of 30).

c. Exemplary Products

An exemplary product that is in the form of liquid and/or wax contains alkylbenzene (e.g. dialkylbenzene), alkylnaphthalene (e.g. dialkylnaphthalene), alkyltetralin (e.g. dialkyltetralin), alkylanthracene, ankylphenanthrene, partially hydrogenated alkylanthracene, and partially hydrogenated ankylphenanthrene. For example, the liquid and/or wax product stream 110 contains alkylbenzene (e.g. dialkylbenzene), alkylnaphthalene (e.g. dialkylnaphthalene), alkyltetralin (e.g. dialkyltetralin), alkylanthracene, ankylphenanthrene, partially hydrogenated alkylanthracene, and partially hydrogenated ankylphenanthrene as shown in FIG. 14.

2. Gas

The process described herein may also produce other compounds that are not in the form of a liquid or wax. For example, depolymerization of the solid waste using the process described herein may also produce a gas. Generally, the gas contains a saturated compound, optionally more than one saturated compound, such as a saturated C₁-C₅ alkane, hydrogen, benzene, or toluene, or a combination thereof.

Optionally, the saturated C₁-C₅ alkane in the produced gas can be linear, branched, or cyclic. For example, the saturated alkane is a linear C₁-C₅ saturated alkane, a branched C₄-C₅ saturated alkane, or a cyclic C₃-C₅ saturated alkane, optionally a linear C₁-C₄ saturated alkane, a branched C₄-C₅ saturated alkane, or a cyclic C₃-C₅ saturated alkane.

For example, the gas produced from depolymerization of the solid waste contains C₁-C₅ saturated alkanes, such as methane, ethane, and propane, hydrogen, benzene, and toluene.

3. Operation Conditions

The reactor is operated at a temperature for a period of time sufficient to convert the hydrocarbon polymer forming the waste material to the product in liquid and/or wax form that contains one or more alkylaromatic compounds.

a. Temperature, Pressure, and Period of Time

Typically, the operation temperature for the reactor is low, for example, the operation temperature does not exceed 500° C. Such low operation temperatures allow energy-efficient depolymerization of waste hydrocarbon polymers.

Optionally, the temperature sufficient to convert the waste hydrocarbon polymer to the product in the form of liquid and/or wax is up to 500° C., up to 450° C., up to 400° C., up to 380° C., up to 350° C., up to 320° C., up to 300° C., up to 290° C., between 250° C. and 500° C., between 250° C. and 450° C., between 250° C. and 400° C., between 320° C. and 500° C., between 320° C. and 450° C., between 320° C. and 400° C., between 350° C. and 500° C., between 350° C. and 450° C., between 350° C. and 400° C., between 250° C. and 360° C., between 250° C. and 350° C., between 250° C. and 330° C., between 250° C. and 320° C., or between 250° C. and 300° C., such as about 250° C., about 280° C., about 330° C., or about 360° C.

The temperature is selected based on the hydrocarbon polymer(s) in the waste material and/or the catalyst. For example, when a Pt/γ-Al₂O₃ catalyst is used for depolymerization of the waste material, the temperature sufficient to convert the hydrocarbon polymer to the liquid and/or wax product is up to 380° C., up to 350° C., up to 320° C., up to 300° C., up to 290° C., between 320° C. and 380° C., between 320° C. and 360° C., between 330° C. and 360° C., between 280° C. and 360° C., between 250° C. and 360° C., between 250° C. and 350° C., between 250° C. and 330° C., between 250° C. and 320° C., or between 250° C. and 300° C., such as about 250° C., about 280° C., about 330° C., or about 360° C. For example, when a Pt/γ-Al₂O₃ catalyst is used for depolymerization of polyethylene, the temperature sufficient to convert the hydrocarbon polymer to the liquid and/or wax product is up to 350° C., up to 320° C., up to 300° C., up to 290° C., between 250° C. and 350° C., between 250° C. and 320° C., or between 250° C. and 300° C., such as about 250° C. or about 280° C.

For example, when a Pt/silica-Al₂O₃ catalyst is used for depolymerization of the waste material, the temperature sufficient to convert the hydrocarbon polymer to the liquid and/or wax product is up to 350° C., up to 320° C., up to 300° C., up to 290° C., between 250° C. and 350° C., between 250° C. and 320° C., between 250° C. and 300° C., or between 250° C. and 280° C., such as about 250° C. For example, when a Pt/silica-Al₂O₃ catalyst is used for depolymerization of polyethylene, the temperature sufficient to convert the hydrocarbon polymer to the liquid and/or wax product is up to 350° C., up to 320° C., up to 300° C., up to 290° C., between 250° C. and 350° C., between 250° C. and 320° C., between 250° C. and 300° C., or between 250° C. and 280° C., such as about 250° C.

For example, when a mixture of Pt/γ-Al₂O₃ and F-γ-Al₂O₃ is used as the catalyst for depolymerization of the waste material, the temperature sufficient to convert the hydrocarbon polymer to the liquid and/or wax product is up to 380° C., up to 350° C., up to 320° C., up to 300° C., up to 290° C., between 320° C. and 380° C., between 320° C. and 360° C., between 330° C. and 360° C., between 280° C. and 360° C., between 250° C. and 360° C., between 250° C. and 350° C., between 250° C. and 330° C., between 250° C. and 320° C., or between 250° C. and 300° C., such as about 250° C., about 280° C., about 330° C., or about 360° C. For example, when a mixture of Pt/γ-Al₂O₃ and F-γ-Al₂O₃ is used as the catalyst for depolymerization of polyethylene, the temperature sufficient to convert the hydrocarbon polymer to the liquid and/or wax product is up to 380° C., up to 350° C., up to 320° C., up to 300° C., up to 290° C., between 320° C. and 380° C., between 320° C. and 360° C., between 330° C. and 360° C., between 280° C. and 360° C., between 250° C. and 360° C., between 250° C. and 350° C., between 250° C. and 330° C., between 250° C. and 320° C., or between 250° C. and 300° C., such as about 250° C., about 280° C., about 330° C., or about 360° C.

Typically, the operation pressure for the reactor to convert the waste hydrocarbon polymer to the product in the form of liquid and/or wax is in a range from 0.01 atm to 50 atm, from 0.1 atm to 50 atm, from 0.5 atm to 40 atm, from 0.1 atm to 40 atm, from 0.1 atm to 30 atm, from 0.1 atm to 20 atm, from 0.1 atm to 10 atm, from 0.1 atm to 5 atm, or from 0.5 atm to 5 atm, such as from 1 atm to 2 atm.

Typically, the period of time sufficient to convert the hydrocarbon polymer to the product is up to 6 hours, up to 5.5 hours, up to 5 hours, up to 4.5 hours, up to 4 hours, up to 3.5 hours, up to 3 hours, up to 2.5 hours, up to 2 hours, up to 1.5 hours, up to 1 hour, up to 55 minutes, up to 50 minutes, up to 45 minutes, up to 40 minutes, up to 35 minutes, up to 30 minutes, up to 25 minutes, up to 20 minutes, up to 15 minutes, up to 10 minutes, up to 5 minutes, in a range from 5 minutes to 6 hours, from 5 minutes to 5.5 hours, from 5 minutes to 5 hours, from 5 minutes to 4.5 hours, from 5 minutes to 4 hours, from 5 minutes to 3.5 hours, from 5 minutes to 3 hours, from 5 minutes to 2.5 hours, from 5 minutes to 2 hours, from 5 minutes to 1.5 hours, from 1 minute to 1 hour, from 5 minutes to 1 hour, from 10 minutes to 1 hour, from 15 minutes to 1 hour, from 20 minutes to 1 hour, from 30 minutes to 1 hour, from 1 minute to 55 minutes, from 1 minute to 50 minutes, from 1 minute to 45 minutes, from 1 minute to 40 minutes, from 1 minute to 35 minutes, or from 1 minute to 30 minutes.

For example, the depolymerization reaction can be performed at a suitable temperature, such as up to 500° C., up to 450° C., up to 400° C., up to 380° C., up to 350° C., up to 320° C., up to 300° C., up to 290° C., between 250° C. and 500° C., between 250° C. and 450° C., between 250° C. and 400° C., between 320° C. and 500° C., between 320° C. and 450° C., between 320° C. and 400° C., between 350° C. and 500° C., between 350° C. and 450° C., between 350° C. and 400° C., between 250° C. and 360° C., between 250° C. and 350° C., between 250° C. and 330° C., between 250° C. and 320° C., or between 250° C. and 300° C., such as about 250° C., about 280° C., about 330° C., or about 360° C., for a suitable period of time, such as from 5 minutes to 6 hours, from 10 minutes to 6 hours, from 20 minutes to 6 hours, from 30 minutes to 6 hours, from 5 minutes to 5 hours, from 10 minutes to 5 hours, from 20 minutes to 5 hours, from 30 minutes to 5 hours, from 5 minutes to 4 hours, from 10 minutes to 4 hours, from 20 minutes to 4 hours, from 30 minutes to 4 hours, from 5 minutes to 3 hours, from 10 minutes to 3 hours, from 20 minutes to 3 hours, from 30 minutes to 3 hours, from 5 minutes to 2 hours, from 10 minutes to 2 hours, from 20 minutes to 2 hours, or from 30 minutes to 2 hours that are sufficient to convert the hydrocarbon polymer to the product in a liquid and/or wax form.

For example, the depolymerization reaction can be performed at a suitable temperature, such as up to 500° C., up to 450° C., up to 400° C., up to 380° C., up to 350° C., up to 320° C., up to 300° C., up to 290° C., between 250° C. and 500° C., between 250° C. and 450° C., between 250° C. and 400° C., between 320° C. and 500° C., between 320° C. and 450° C., between 320° C. and 400° C., between 350° C. and 500° C., between 350° C. and 450° C., between 350° C. and 400° C., between 250° C. and 360° C., between 250° C. and 350° C., between 250° C. and 330° C., between 250° C. and 320° C., or between 250° C. and 300° C., such as about 250° C., about 280° C., about 330° C., or about 360° C., under a suitable pressure, such as from 0.01 atm to 50 atm, from 0.1 atm to 50 atm, from 0.5 atm to 40 atm, from 0.1 atm to 40 atm, from 0.1 atm to 30 atm, from 0.1 atm to 20 atm, from 0.1 atm to 10 atm, from 0.1 atm to 5 atm, from 0.5 atm to 5 atm, or from 1 atm to 2 atm, for a suitable period of time, such as from 5 minutes to 6 hours, from 10 minutes to 6 hours, from 20 minutes to 6 hours, from 30 minutes to 6 hours, from 5 minutes to 5 hours, from 10 minutes to 5 hours, from 20 minutes to 5 hours, from 30 minutes to 5 hours, from 5 minutes to 4 hours, from 10 minutes to 4 hours, from 20 minutes to 4 hours, from 30 minutes to 4 hours, from 5 minutes to 3 hours, from 10 minutes to 3 hours, from 20 minutes to 3 hours, from 30 minutes to 3 hours, from 5 minutes to 2 hours, from 10 minutes to 2 hours, from 20 minutes to 2 hours, or from 30 minutes to 2 hours that are sufficient to convert the hydrocarbon polymer to the product in a liquid and/or wax form.

b. Atmosphere

Optionally, the depolymerization reaction is operated in the reactor in an atmosphere filled with an inert gas. Exemplary inert gas includes, but is not limited to, argon, nitrogen, helium, neon, krypton, xenon, and radon. For example, the depolymerization reaction is operated in an atmosphere filled with argon or nitrogen.

In some embodiments, the depolymerization reaction is operated in an atmosphere that is hydrogen gas-free.

4. Characterization of the Depolymerization Process

The disclosed process for depolymerizing a solid waste containing hydrocarbon polymer(s) can be characterized by product yield and selectivity to the unsaturated compounds.

a. Yield

Typically, the liquid and/or wax product has a yield of at least 50 wt %, at least 55 wt %, at least 60 wt %, at least 65 wt %, at least 70 wt %, at least 75 wt %, at least 80 wt %, at least 85 wt %, at least 90 wt %, at least 95 wt %, between 50 wt % and 95 wt %, between 60 wt % and 95 wt %, between 70 wt % and 95 wt %, or between 75 wt % and 95 wt %.

Optionally, the alkylaromatic compound(s) in the liquid and/or wax product has a total yield of at least 40 wt %, at least 45 wt %, at least 50 wt %, at least 55 wt %, at least 60 wt %, at least 65 wt %, at least 70 wt %, at least 75 wt %, at least 80 wt %, between 40 wt % and 85 wt %, between 45 wt % and 85 wt %, between 50 wt % and 85 wt %, between 55 wt % and 85 wt %, or between 60 wt % and 85 wt %. The term “total yield” refers to the weight of the alkylaromatic compound or the total weight of the alkylaromatic compounds when the product contains more than one alkylaromatic compound, relative to the total weight of the hydrocarbon polymer(s) of the waste material.

b. Selectivity

The process disclosed herein may produce other compounds that are not alkylaromatic compounds, such as other unsaturated compounds, alkanes, and/or a gas. For example, depolymerization of polyethylene using the disclosed process produces alkylaromatic compound(s), such as one or more alkylaromatic compounds described above and/or one or more other unsaturated compounds described above in the liquid and/or wax product.

The selectivity of the disclosed process to convert the hydrocarbon polymer to the alkylaromatic compound(s) described above can be measured by NMR, such as ¹H NMR or ¹³C NMR. Exemplary methods for determining the selectivity to the alkylaromatic compound(s) in the liquid and/or wax product, are shown in Example 1.

Typically, the disclosed process is able to convert the hydrocarbon polymer to the liquid and/or wax product with a total selectivity to the alkylaromatic compound(s) described above of at least 25 mol %, at least 30 mol %, at least 35 mol %, at least 40 mol %, at least 45 mol %, at least 50 mol %, at least 55 mol %, at least 60 mol %, at least 65 mol %, at least 70 mol %, at least 75 mol %, at least 80 mol %, at least 85 mol %, at least 90 mol %, at least 95 mol %, between 25 mol % and 95 mol %, between 30 mol % and 95 mol %, between 35 mol % and 95 mol %, between 40 mol % and 95 mol %, between 50 mol % and 95 mol %, or between 60 mol % and 90 mol %. The term “total selectivity to the alkylaromatic compound(s)” refers to the total moles of the one or more alkylaromatic compounds in the product relative to the total moles of the liquid and/or wax product. For example, the disclosed process to convert the hydrocarbon polymer to the product has a total selectivity to alkylaromatic compounds including alkylbenzene, alkyltetralin, alkylnaphthalene, alkylanthracene, alkylphenanthrene, a partially hydrogenated alkyanthracene, and a partially hydrogenated alkylphenathrene, of at least 50 mol %, at least 55 mol %, at least 60 mol %, at least 65 mol %, at least 70 mol %, at least 75 mol %, at least 80 mol %, at least 85 mol %, at least 90 mol %, at least 95 mol %, between 50 mol % and 95 mol %, or between 60 mol % and 90 mol %. For example, the disclosed process to convert the hydrocarbon polymer to the product has a total selectivity to alkylaromatic compounds including alkylbenzene and alkylnaphthalene, of at least 35 mol %, at least 40 mol %, at least 45 mol %, at least 50 mol %, between 35 mol % and 95 mol %, or between 35 mol % and 90 mol %.

For example, as shown in FIG. 14, the mol % of the alkylaromatic compound, optionally the total mol % of the more than one alkylaromatic compound, in the liquid and/or wax product stream 110 can be at least 50 mol %, at least 55 mol %, at least 60 mol %, at least 65 mol %, at least 70 mol %, at least 75 mol %, at least 80 mol %, at least 85 mol %, at least 90 mol %, at least 95 mol %, between 50 mol % and 95 mol %, or between 60 mol % and 90 mol %.

Optionally, the disclosed process to convert the hydrocarbon polymer to the product has a total selectivity to the monoaromatic compound(s) of at least 10 mol %, at least 20 mol %, at least 25 mol %, at least 30 mol %, at least 35 mol %, at least 40 mol %, at least 45 mol %, at least 50 mol %, at least 55 mol %, at least 60 mol %, at least 65 mol %, at least 70 mol %, between 40 mol % and 70 mol %, between 10 mol % and 60 mol %, between 10 mol % and 40 mol %, between 20 mol % and 60 mol %, between 20 mol % and 40 mol %, between 40 mol % and 60 mol %, or between 45 mol % and 60 mol %. The term “total selectivity to the alkylbenzene(s)” refers to the total mole of the one or more alkylbenzene(s) in the product relative to the total mole of the product. For example, the disclosed process to convert the hydrocarbon polymer to the product has a total selectivity to alkylbenzene(s) and alkyltetralin(s), of at least 10 mol %, at least 20 mol %, at least 25 mol %, at least 30 mol %, at least 35 mol %, at least 40 mol %, at least 45 mol %, at least 50 mol %, at least 55 mol %, at least 60 mol %, at least 65 mol %, at least 70 mol %, between 20 mol % and 60 mol %, between 20 mol % and 40 mol %, between 40 mol % and 70 mol %, between 40 mol % and 60 mol %, or between 45 mol % and 60 mol %.

Optionally, the disclosed process to convert the hydrocarbon polymer to the product has a total selectivity to the polycyclic aromatic compound(s) of at least 20 mol %, at least 25 mol %, at least 30 mol %, at least 35 mol %, at least 40 mol %, at least 45 mol %, at least 50 mol %, at least 55 mol %, at least 60 mol %, at least 65 mol %, at least 70 mol %, between 40 mol % and 70 mol %, between 20 mol % and 60 mol %, between 20 mol % and 40 mol %, between 20 mol % and 30 mol %, between 40 mol % and 60 mol %, or between 45 mol % and 60 mol %. For example, the disclosed process to convert the hydrocarbon polymer to the product has a total selectivity to alkylnaphthalene(s) of at least 20 mol %, at least 25 mol %, at least 30 mol %, at least 35 mol %, at least 40 mol %, at least 45 mol %, at least 50 mol %, at least 55 mol %, at least 60 mol %, at least 65 mol %, at least 70 mol %, between 20 mol % and 60 mol %, between 20 mol % and 40 mol %, between 20 mol % and 30 mol %, between 40 mol % and 70 mol %, between 40 mol % and 60 mol %, or between 45 mol % and 60 mol %. The term “total selectivity to the alkylnaphthalene(s)” refers to the total mole of the one or more alkylnaphthalene(s) in the product relative to the total mole of the product.

C. Optional Steps

1. Processing the Waste Hydrocarbon Polymer

The process optionally includes a step of processing the waste material containing one or more hydrocarbon polymers to a suitable form prior to step (i) or subsequent to step (i) and prior to step (ii). Generally, the waste material containing one or more hydrocarbon polymer(s) is processed to a form that improves the contact between the polymer and the catalyst.

For example, the waste material is in the form of a solid and the solid waste is processed by shredding, cutting, and/or grinding the waste hydrocarbon polymer to small parts prior to feeding it into the reactor, such that the contact between processed solid waste and the catalyst is improved compared with the solid waste prior to processing.

Optionally, the waste material is in the form of a solid and the solid waste is dissolved in a solvent, optionally in a hydrocarbon solvent, prior to feeding it into the reactor or the waste is dissolved in the solvent in the reactor after feeding it into the reactor and prior to the depolymerization reaction, such that the contact between the dissolved solid waste and the catalyst is improved compared with the solid waste without dissolving. For example, the solvent is fed into the reactor, and the solid waste is subsequently fed into the reactor and dissolved in the solvent. Alternatively, the solid waste is fed into the reactor and the solvent is subsequently fed into the reactor and dissolves the solid waste. Optionally, the solid waste and the solvent are fed into the reactor simultaneously or substantially simultaneously, and the solid waste is dissolved in the solvent in the reactor.

Typically, the hydrocarbon solvent is a small hydrocarbon solvent, i.e. a hydrocarbon solvent containing less than 11 carbon atoms. Suitable hydrocarbon solvents for dissolving the solid waste include, but are not limited to, hexane, cyclohexane, isopentane, a mixture of n-pentane and isopentane (e.g. pentane 60/40 and pentane 80/20), toluene, TOPSol (e.g. TOPSol 60/145, TOPSol A100, TOPSol A150, TOPSol A150ND, TOPSol BF, TOPSol X2000, and TOPSol 2046), a mixture of paraffins, cycliparaffins, and aromatics (WS 200), xylene, benzene, ethylbenzene, and a mixture of xylene and ethylbenzene.

For example, as shown in FIG. 14, a solvent can be fed into the reactor 10 via a reagent stream 200 prior to feeding the waste material, or subsequent to feeding the waste material and prior to the depolymerization reaction, or simultaneously or substantially simultaneously with feeding the waste material, such that the solid waste is dissolved in the solvent in the reactor.

2. Cooling the Reactor to Room Temperature

The process optionally includes a step of cooling the reactor to room temperature after step (ii). The reactor may be cooled by any suitable method. For example, the reactor can be cooled in an air-flow or in a water bath of room temperature (i.e. 25° C.).

3. Recycling the Catalyst

The method may include a step of recycling the catalyst after step (ii). Typically, the catalyst can be recycled and reused for the depolymerization reaction without significant loss of activity. For example, the catalyst can be recycled and reused for the depolymerization reaction with less than 5%, less than 10%, or less than 20% decrease in product yield. Optionally, the selectivity of the process to form the one or more unsaturated compounds using a recycled catalyst is the same or substantially the same as that using a fresh catalyst. The catalyst can be recycled and reused at least 2 times, at least 3 times, at least 4 times, at least 5 times, at least 6 times, at least 7 times, at least 8 times, at least 9 times, or at least 10 times.

Catalyst may be recycled by oxidation with an oxidizing gas and then optionally reduced with a reducing gas.

a. Oxidation

After step (ii), the catalyst is recycled. For example, the catalyst is exposed to an oxidizing gas to oxidize the catalyst. Examples of suitable oxidizing gas for oxidizing the catalyst include, but are not limited to, oxygen and air. For example, the oxidizing gas is oxygen.

Generally, the catalyst is oxidized with an oxidizing gas at a temperature at least 200° C., at least 250° C., at least 300° C., up to 450° C., up to 400° C., from 200° C. to 450° C., from 250° C. to 450° C., or from 300° C. to 450° C., such as from 350° C. to 450° C.

Generally, the catalyst is oxidized with the oxidizing gas for a time period at least 10 minutes, at least 20 minutes, at least 30 minutes, at least 40 minutes, at least 50 minutes, at least 1 hour, up to 5 hours, up to 4 hours, up to 3 hours, up to 2 hours, between 10 minutes and 2 hours, between 30 minutes and 2 hours, or between 1 hour and 2 hours.

For example, the oxidation is performed at a temperature at least 200° C., at least 250° C., at least 300° C., up to 450° C., up to 400° C., from 200° C. to 450° C., from 250° C. to 450° C., or from 300° C. to 450° C., for a time period at least 10 minutes, at least 20 minutes, at least 30 minutes, at least 40 minutes, at least 50 minutes, at least 1 hour, up to 5 hours, up to 4 hours, up to 3 hours, up to 2 hours, between 10 minutes and 2 hours, between 30 minutes and 2 hours, or between 1 hour and 2 hours. For example, the oxidation is performed at a temperature from 250° C. to 450° C. for a time period between 10 minutes and 2 hours or between 30 minutes and 2 hours.

b. Reduction

Optionally, after oxidation the oxidized catalyst is reduced with a reducing gas. Examples of suitable reducing gas for reducing the oxidized catalyst include, but are not limited to, hydrogen, carbon monoxide, ammonia, methane, and nitric oxide. For example, the reducing gas is hydrogen.

The reduction temperature can be at least 150° C., at least 200° C., at least 250° C., up to 400° C., up to 350° C., up to 300° C., between 150° C. and 400° C., between 150° C. and 350° C., or between 150° C. and 300° C., such as between 200° C. and 300° C. Typically, the reduction temperature is lower than the oxidation temperature. For example, the catalyst is oxidized at 400° C. and then reduced at 250° C.

The period of time for the reduction step can be the same, substantially the same, or different than the period of time for the oxidation step.

For example, the reduction step is performed at a temperature at least 150° C., at least 200° C., at least 250° C., up to 400° C., up to 350° C., up to 300° C., between 150° C. and 400° C., between 150° C. and 350° C., or between 150° C. and 300° C. for a period of time at least 10 minutes, at least 20 minutes, at least 30 minutes, at least 40 minutes, at least 50 minutes, at least 1 hour, up to 5 hours, up to 4 hours, up to 3 hours, up to 2 hours, between 10 minutes and 2 hours, between 30 minutes and 2 hours, or between 1 hour and 2 hours. For example, the oxidation is performed at a temperature from 250° C. to 450° C. for a time period between 10 minutes and 2 hours or between 30 minutes and 2 hours, and the reduction is performed at a temperature between 200° C. and 300° C. for a time period between 30 minutes and 3 hours or between 1 hour and 3 hours.

Optionally, the period of time for the reduction step is different than the period of time for the oxidation step. The period of time for the reduction step may be longer or shorter than the period of time for the oxidation step. For example, the period of time for the oxidation step is 2 hours, and the period of time for the reduction step is 3 hours.

4. Separating the Liquid and/or Wax Product

The upcycling process optionally includes a step of separating the liquid and/or wax product subsequent to step (ii). For example, as shown in FIG. 14, the liquid and/or wax product stream 110 enters into a separation unit 20 and is separated into two or more streams. In FIG. 14, the liquid and/or wax product stream 110 enters into separation unit 20 and is separated into three streams: an alkylaromatic compound stream 113, an alkane stream 112 (i.e. a mixture of alkanes with carbon numbers in a range from 11 to 80, thus the alkane stream contains alkanes with an average carbon number of 30), and an C₆-C₁₀ alkane and aromatic stream 111.

Suitable separators that can be used for the step of separating the liquid and/or wax product are known. For example, the liquid and/or wax product can be separated by distillation as described in U.S. Pat. No. 2,848,387 to Glazier, et al. and U.S. Pat. No. 3,308,060 to Ellis.

5. Feeding Catalyst into the Reactor

The process optionally includes a step of feeding the catalyst into the reactor prior to step (i), or subsequent to step (i) and prior to step (ii), or simultaneously or substantially simultaneously with step (i). The catalyst in the reactor is in contact with the hydrocarbon polymer of the waste material. For example, as shown in FIG. 14, the catalyst is fed into the reactor 10 via a catalyst stream 200 prior to feeding the waste material, or subsequent to feeding the waste material and prior to the depolymerization reaction, or simultaneously or substantially simultaneously with feeding the waste material.

The disclosed methods can be further understood through the following enumerated paragraphs.
1. A process for upcycling a waste material, wherein the waste material comprises a hydrocarbon polymer, optionally more than one hydrocarbon polymer, comprising
   (i) feeding the waste material into a reactor,
      wherein the reactor comprises a catalyst therein,
         wherein the catalyst comprises a transition metal; and
   (ii) operating the reactor at a sufficient temperature for a sufficient period of time to convert the hydrocarbon polymer to a product in the form of a liquid and/or a wax comprising an alkylaromatic compound, optionally more than one alkylaromatic compound,
   wherein the mol % of the alkylaromatic compound, optionally the total mol % of the more than one alkylaromatic compound, in the product is at least 50 mol %.
2. The process of paragraph 1, wherein the hydrocarbon polymer is polyethylene, polypropylene, polystyrene, a copolymer of polyethylene and polypropylene, or acrylonitrile butadiene styrene.
3. The process of paragraph 1 or paragraph 2, wherein the alkylaromatic compound contains from 10 to 50 carbon atoms, from 12 to 50 carbon atoms, from 15 to 50 carbon atoms, from 15 to 25 carbons, from 20 to 40 carbons, or from 20 to 35 carbon atoms.
4. The process of any one of paragraphs 1-3, wherein the alkylaromatic compound has a weight average molecular weight (Mw) in a range from 150 g mol⁻¹ to 800 g mol⁻¹, from 200 g mol⁻¹ to 800 g mol⁻¹, from 200 g mol⁻¹ to 600 g mol⁻¹, from 200 g mol⁻¹ to 500 g mol⁻¹, from 300 g mol⁻¹ to 800 g mol-1, from 300 g mol⁻¹ to 600 g mol⁻¹, or from 300 g mol⁻¹ to 500 g mol⁻¹.
5. The process of any one of paragraphs 1-4, wherein the product further comprises an olefin.
6. The process of any one of paragraphs 1-5, wherein the alkylaromatic compound is a dialkylbenzene, an alkyltetralin, a dialkylnaphthalene, an alkylanthracene, an alkylphenanthrene, a partially hydrogenated alkyanthracene, or a partially hydrogenated alkylphenathrene, or a combination thereof.
7. The process of paragraph 6, wherein the dialkylbenezene has a structure of Formula (I):

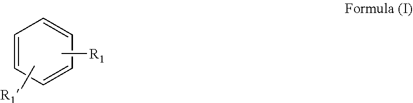

wherein R₁ and R₁′ are independently a C₁-C₂₀ alkyl group, a C1-C₁₅ alkyl group, a C₁-C₁₂ alkyl group, a C₁-C₁₀ alkyl group, or a C₁-C₅ alkyl group, and
wherein the sum of the carbon atoms in R₁ and R₁′ is at least 10.
8. The process of paragraph 6, wherein the dialkylnaphthalene has a structure of Formula (II):

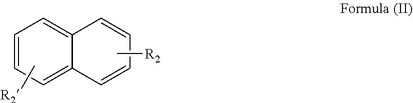

wherein R₂ and R₂′ are independently a C₁-C₂₀ alkyl group, a C1-C₁₅ alkyl group, a C1-C12 alkyl group, a C₁-C₁₀ alkyl group, or a C1-C₅ alkyl group, and
wherein the sum of the carbon atoms in R₂ and R₂′ is at least 4, optionally at least 5.
9. The process of any one of paragraphs 1-8, wherein the transition metal is selected from the group consisting of platinum, palladium, ruthenium, iridium, rhenium, rhodium, iron, cobalt, nickel, copper, molybdenum, and tungsten.
10. The process of any one of paragraphs 1-9, wherein the catalyst comprises more than one transition metal, wherein each of the transition metals is selected from the group consisting of platinum, palladium, ruthenium, iridium, rhenium, rhodium, iron, cobalt, nickel, copper, molybdenum, and tungsten.
11. The process of any one of paragraphs 1-9, wherein the catalyst comprises more than one transition metal, wherein each of the transition metals is selected from the group consisting of platinum, palladium, ruthenium, iridium, rhodium, iron, cobalt, nickel, copper, molybdenum, and tungsten; and wherein the catalyst further comprises a second metal, wherein the second metal is different from each of the transition metals, and wherein the second metal is selected from the group consisting of rhenium, tin, lead, tungsten, molybdenum, chromium, manganese, and zinc, or a combination thereof
12. The process of any one of paragraphs 1-8, wherein the catalyst is a metal, a mixture of two or more metals comprising the transition metal, a metal oxide of the transition metal, or a metal carbide of the transition metal, or a combination thereof.
13. The process of any one of paragraphs 1-12, wherein the catalyst is dispersed on a surface of a first support and wherein the catalyst is in the form of atoms, nanoclusters, or nanoparticles, or a combination thereof.
14. The process of paragraph 13, wherein the catalyst is platinum nanoparticles, wherein the first support is an aluminum oxide support or silica-aluminum oxide support, and wherein the platinum nanoparticles are dispersed on the surface of the aluminum oxide support or silica-aluminum oxide support.
15. The process of paragraph 13 or 14, further comprising a second support, wherein the first support having the catalyst dispersed thereon is mixed with the second support and the second support is an acidic support.
16. The process of paragraph 15, wherein the second support is a halogenated alumina support, optionally a fluorinated alumina support.
17. The process of any one of paragraphs 13-16, wherein the total weight loading of the metal is in a range from 0.1 wt % to 10 wt %, from 0.1 wt % to 8 wt %, from 0.1 wt % to 5 wt %, from 0.1 wt % to 1 wt %, from 0.5 wt % to 10 wt %, from 0.5 wt % to 8 wt %, from 0.5 wt % to 5 wt %, from 1 wt % to 10 wt %, from 1 wt % to 8 wt %, or from 1 wt % to 5 wt % of the total weight of the catalyst and the first support or the total weight of the catalyst and the first and second support.
18. The process of any one of paragraphs 15-17, wherein the weight percentage of the second support is in a range from 2% to 90%, from 2% to 50%, from 2% to 35%, from 10% to 90%, from 10% to 80%, from 10% to 70%, from 10% to 60%, from 10% to 50%, from 10% to 30%, from 15% to 40%, from 20% to 50%, from 20% to 40%, or from 20% to 35% of the total weight of the catalyst and the first and second support.
19. The process of any one of paragraphs 1-18, wherein during step (ii), the reactor is operated at a temperature of up to 500° C., up to 450° C., up to 400° C., up to 350° C., up to 320° C., up to 300° C., up to 290° C., between 250° C. and 500° C., between 250° C. and 450° C., between 250° C. and 400° C., between 300° C. and 500° C., between 320° C. and 500° C., between 300° C. and 450° C., between 320° C. and 450° C., between 300° C. and 400° C., between 300° C. and 360° C., between 250° C. and 350° C., between 250° C. and 350° C., between 250° C. and 320° C., or between 250° C. and 300° C.
20. The process of any one of paragraphs 1-19, wherein during step (ii), the reactor is operated for a period of time of up to 6 hours, up to 5.5 hours, up to 5 hours, up to 4.5 hours, up to 4 hours, up to 3.5 hours, up to 3 hours, up to 2.5 hours, up to 2 hours, up to 1.5 hours, up to 1 hour, up to 55 minutes, up to 50 minutes, up to 45 minutes, up to 40 minutes, up to 35 minutes, up to 30 minutes, up to 25 minutes, up to 20 minutes, up to 15 minutes, up to 10 minutes, up to 5 minutes, in a range from 5 minutes to 6 hours, from 5 minutes to 5.5 hours, from 5 minutes to 5 hours, from 5 minutes to 4.5 hours, from 5 minutes to 4 hours, from 5 minutes to 3.5 hours, from 5 minutes to 3 hours, from 5 minutes to 2.5 hours, from 5 minutes to 2 hours, from 5 minutes to 1.5 hours, from 1 minute to 1 hour, from 5 minutes to 1 hour, from 10 minutes to 1 hour, from 15 minutes to 1 hour, from 20 minutes to 1 hour, from 30 minutes to 1 hour, from 1 minute to 55 minutes, from 1 minute to 50 minutes, from 1 minute to 45 minutes, from 1 minute to 40 minutes, from 1 minute to 35 minutes, or from 1 minute to 30 minutes.
21. The process of any one of paragraphs 1-20, wherein following steps (i) and (ii), the mol % of the alkylaromatic compound, optionally the total mol % of the more than one alkylaromatic compound, in the product is at least 30 mol %, at least 35 mol %, at least 40 mol %, at least 45 mol %, at least 50 mol %, at least 55 mol %, at least 60 mol %, at least 65 mol %, at least 70 mol %, at least 75 mol %, at least 80 mol %, at least 85 mol %, at least 90 mol %, at least 95 mol %, between 30 mol % and 95 mol %, between 35 mol % and 95 mol %, between 50 mol % and 95 mol %, or between 60 mol % and 90 mol %.
22. The process of any one of paragraphs 1-21, wherein following steps (i) and (ii), the liquid and/or wax product has a yield of at least 70 wt %, at least 75 wt %, at least 80 wt %, at least 85 wt %, at least 90 wt %, at least 95 wt %, between 70 wt % and 95 wt %, or between 75 wt % and 95 wt %.
23. The process of any one of paragraphs 1-22, wherein following steps (i) and (ii), the alkylaromatic compound has a yield of at least 40 wt %, at least 45 wt %, at least 50 wt %, at least 55 wt %, at least 60 wt %, at least 65 wt %, at least 70 wt %, at least 75 wt %, at least 80 wt %, between 40 wt % and 85 wt %, between 45 wt % and 85 wt %, between 50 wt % and 85 wt %, between 55 wt % and 85 wt %, or between 60 wt % and 85 wt.
24. The process of any one of paragraphs 1-23, wherein the hydrocarbon polymer is a high density polyethylene polymer or a low density polyethylene polymer, or a combination thereof.
25. The process of any one of paragraphs 1-24, wherein following steps (i) and (ii), the distribution of the molecular weight of the alkylaromatic compound has a dispersity of less than 3.5, less than 3.0, less than 2.5, less than 2.0, less than 1.5, less than 1.3, less than 1.2, between 1 and 1.5, between 1 and 1.4, between 1 and 1.3, or between 1 and 1.2, between 1.1 and 1.5, between 1.1 and 1.4, between 1.1 and 1.3, or between 1.1 and 1.2.
26. The process of any one of paragraphs 1-25, further comprising a step of (iii) processing the waste to a suitable form prior to step (i).
27. The process of paragraph 26, wherein the waste is in the form of solid waste, and wherein step (iii) includes shredding, cutting, and/or grinding the waste hydrocarbon polymer to small parts.
28. The process of any one of paragraphs 1-27, wherein the waste is in the form of solid waste, and wherein the process further comprises, prior to step (i), a step of dissolving the solid waste in a solvent, optionally in a hydrocarbon solvent.
29. The process of any one of paragraphs 1-28, further comprising a step of (iv) cooling the reactor to room temperature after step (ii).
30. The process of any one of paragraphs 1-29, further comprising a step of (v) recycling the catalyst after step (ii).
31. A composition comprising an alkylaromatic compound, optionally more than one alkylaromatic compound, wherein the composition is in the form of a liquid and/or wax, and wherein the composition is produced by the process of any one of paragraphs 1 to 30.
32. The process of paragraph 16, wherein the weight percentage of the halogen, optionally the fluorine, in the second support is in a range from 0.1% to 5%, from 0.5% to 3%, from 0.5% to 2%, from 0.5% to 1.5%, optionally about 0.7% or about 1.3%.
33. The process of paragraph 16 or 32, wherein the weight percentage of the second support is in a range from 2% to 35%, from 5% to 35%, from 10% to 35%, or from 10% to 30% of the total weight of the catalyst and the first and second support.
34. The process of any one of paragraphs 16, 32, and 33, wherein during step (ii), the reactor is operated at a temperature of up to 350° C., up to 320° C., up to 300° C., in a range from 250° C. to 350° C., from 250° C. to 350° C., from 250° C. to 320° C., or from 250° C. to 300° C., optionally about 280° C.
35. The process of any one of paragraphs 16 and 32-34, wherein during step (ii), the reactor is operated for a period of time of up to 2.5 hours, up to 2 hours, up to 1.5 hours, up to 1 hour, in a range from 5 minutes to 2.5 hours, from 5 minutes to 2 hours, from 5 minutes to 1.5 hours, from 1 minute to 1 hour, from 5 minutes to 1 hour, from 10 minutes to 1 hour, from 15 minutes to 1 hour, from 20 minutes to 1 hour, or from 30 minutes to 1 hour.
36. The process of any one of paragraphs 16 and 32-35, wherein following steps (i) and (ii), the mol % of the alkylaromatic compound, optionally the total mol % of the more than one alkylaromatic compound, in the product is at least 25 mol %, at least 30 mol %, at least 40 mol %, in a range from 25 mol % to 95 mol %, from 30 mol % to 95 mol %, or from 40 mol % to 95 mol %.
37. The process of any one of paragraphs 16 and 32-36, wherein following steps (i) and (ii), the liquid and/or wax product has a yield of at least 50 wt %, at least 55 wt %, at least 60 wt %, at least 65 wt %, in a range from 50 wt % to 95 wt %, or from 60 wt % to 95 wt %.
38. A composition comprising an alkylaromatic compound, optionally more than one alkylaromatic compound, wherein the composition is in the form of a liquid and/or wax, and wherein the composition is produced by the process of any one of paragraphs 32 to 37.

The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The present invention will be further understood by reference to the following non-limiting examples. Although the Examples described below are bench scale reactions, the conditions can be scaled up for use in industrial scale reactors to produce alkylaromatics with the yield and selectivity that are similar to or substantially similar to those produced in the bench scale reactions described below.

### EXAMPLES

### Example 1. Pt/Al₂O₃ Catalyst Converts Polyethylene to Alkylaromatics with High Selectivity at Low Temperature

Materials and Methods

Materials

Trimethyl(cyclopentadienyl)platinum (CpPtMe₃, 99%) and γ-alumina (Lot 29481900, 186 m² g⁻¹, pore volume 0.50 cm³ g⁻¹) were both purchased from Strem. Chloroform (HPLC, OmniSolv®, CX1054-6) was obtained from EMD Millipore Corp. HPLC-grade water, dichloromethane (≥99.9%, GC Resolv™), triethylamine (HPLC-grade) and 1,2,4-trichlorobenzene (HPLC-grade) were obtained from Fisher Chemicals. H₂ (4.0% in Ar, Airgas) was purified by passage through 13× molecular sieves and BTS catalyst (Sigma Aldrich) before use. H₂ (10.0% in Ar, Airgas) was used for calibration of the TCD response. Propene (99.8%) was obtained from PRAXAir. 1,1,2,2-Tetrachloroethane-d₂ (TCE-d₂, ≥99.5 atom % D), KBr (FT-IR grade), triacontane (n-C₃₀H₆₂, 98%), dodecylbenzene (97%) 1,4-didodecylbenzene (95%), di-tert-butylhydroxytoluene (>99%), and chloroplatinic acid hexahydrate (≥37.5 wt % Pt) were all purchased from Sigma Aldrich and used as received. Dichloromethane-d₂ (99.8 atom % D) was purchased from Cambridge Isotope Laboratories. A standard mixture of saturated alkanes (C₇-C₄₀, certified reference material, 1000 μg/mL each component in hexane) and a standard Pt solution containing (976±2) mg Pt/kg in 5% HCl and high-purity water were obtained from Sigma Aldrich. Tetraammineplatinum (II) nitrate (Pt(NH₃)₄(NO₃)₂, >50.0% Pt basis) was purchased from Sigma Aldrich. Amorphous silica-alumina (ASA, Davicat 3113, 7.6 wt % Al, 573 m² g⁻¹) was purchased from Grace-Davison.

Catalyst Synthesis and Characterization

Synthesis of Pt/γ-Alumina

γ-Alumina was calcined in flowing air at 500° C. for 4 h, followed by evacuation (10⁻⁴ Torr) at 450° C. for 12 h. Volatile trimethyl(cyclopentadienyl)platinum (32±1 mg) was deposited onto the dry alumina (1.000±0.020 g) by vacuum sublimation (ca. 10⁻⁴ Torr) at room temperature (Gozum, et al., J. Am. Chem. Soc. 110, 2688-2689 (1988); Garcia, et al., Mater. Trans. 44, 1717-1728 (2003)). The reactor was shaken vigorously during the procedure to promote uniform deposition, then was evacuated at room temperature for 1 h to remove physiosorbed PtCp(CH₃)₃. The resulting solid was reduced in flowing H₂ (4.0% in Ar, 30 mL/min) as the temperature was ramped to 250° C. at a rate of 2° C./min. The reactor was held at this temperature for 2 h, then cooled to room temperature and evacuated for 15 min. The reduced catalyst was stored in an Ar-filled glovebox until use to avoid re-oxidation in air.

Synthesis of Pt/Silica-Alumina

˜1 wt % Pt/silica-alumina was prepared by incipient wet impregnation of 1.0 g silica-alumina with 0.5 ml aqueous solution containing 20 mg of Pt(NH₃)₄(NO₃)₂, followed by drying at 80° C. overnight and finally calcination at 400° C. for 3 h under static air. The catalyst was then reduced under 4% H₂ in Ar at 350° C. for 2 h and then stored in the Ar-filled glovebox before use.

Synthesis of F-γ-Al₂O₃

1.5 g of calcined γ-Al₂O₃ was impregnated with 0.6 ml aqueous solution containing 87.6 mg of NH₄F, followed by drying in the oven at 80° C. overnight and calcination at 550° C. for 5 hours under static air. The resulting materials were then evacuated under ca. 10⁻⁴ Torr at 400° C. for 8 hours. The fresh synthesized catalyst was stored in an Ar-filled glovebox before use or characterization. The fluorine content was determined to be 1.3 wt % based on elemental analysis, which was performed by flask combustion followed by chromatography. A lower content of fluorine on γ-Al₂O₃ was synthesized using the same procedure with half the amount of NH₄F, giving 0.7 wt % F-γ-Al₂O₃ based on elemental analysis.

Characterization

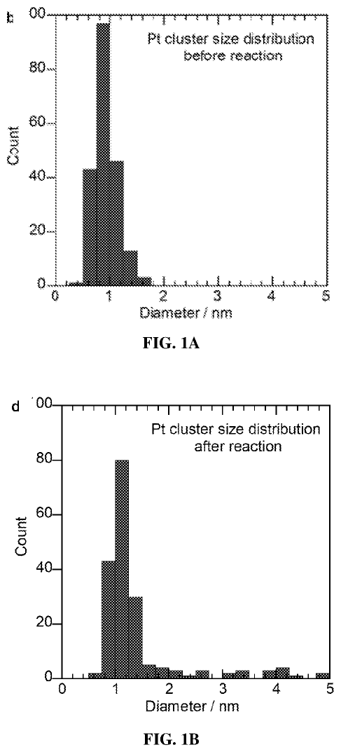

TEM images of the catalyst were obtained on either a FEI Titan 80-300 kV S/TEM or ThermoFisher Talos F200X. Pt particle sizes were measured from the high angle annular dark field (HAADF) images. The fresh catalyst contains particles with an average size of (0.9±0.2) nm (FIG. 1A). After the catalytic disassembly of PE at 280° C. for 24 h, the average size increased slightly, to (1.2±0.3) nm (FIG. 1B).

Metal analyses were performed by Inductively-Coupled Plasma (ICP)—Optical Emission Spectrometry (ICP-OES) on a Thermo iCAP 6300. A calibration curve was constructed using standard Pt solutions, prepared using a commercial Pt standard with a mixture of aqua regia in HPLC-grade water (4% v:v). Pt was extracted from solid catalyst samples at 75° C. in aqua regia under magnetic stirring for 30 min, based on a slight modification of a previously described procedure (Marinho, et al., J. Hazard. Mater. 192, 1155-1160 (2011)). The resulting suspensions were allowed to stand at room temperature for 24 h, then filtered. The solid residue was washed three times with HPLC-grade H₂O. The combined filtrates were diluted with aqua regia (4% v:v in water) and analyzed by ICP-OES, using the spectral lines at 214.423 and 203.646 nm. The fresh catalyst contains 1.5 wt % Pt.

Procedures for Catalytic Reactions

Initial screening reactions and time-resolved experiments were conducted in unstirred stainless-steel mini-autoclaves made from Swagelok junctions (Macala, et al., ChemSusChem. 2, 215-217 (2009)), each with an internal volume of ca. 10 mL. In a typical procedure, the reduced catalyst (1.5 wt % Pt/γ-Al₂O₃, 0.200 g) and a sample of the appropriate PE material (0.118 g) were loaded into an autoclave reactor inside an Ar-filled glove-box. The reactor was sealed, removed from the glove-box and placed in a temperature-controlled muffle furnace (Thermo Scientific Lindberg/Blue M BF51848C) that was pre-heated to the desired temperature. When the furnace door was opened to insert the autoclave, the temperature decreased by 20-30° C., but returned to the desired temperature within ca. 5 min. Reaction timing started when the temperature stabilized. During the 24 h reaction time, the furnace temperature fluctuated by ±5° C. After the specified reaction time, the reactor was removed from the oven and cooled rapidly in a jet of compressed air to quench the reaction.

At the end of the reaction, the reactor was opened inside a fume hood and the gases were vented. The remaining material was transferred onto a fine glass frit (4.0-5.5 μm), washed with hot (50° C.) chloroform, and filtered to remove insolubles. Soluble hydrocarbons were recovered by evaporating the chloroform under reduced pressure (0.1 Torr) overnight. The insoluble material, including the catalyst and hydrocarbons insoluble in hot chloroform, was recovered from the frit. After removing the chloroform residue by evacuating under reduced pressure (0.1 Torr) overnight, the insoluble material was weighed. The yield of insoluble hydrocarbons was calculated as the difference between the total mass of recovered solid and the initial mass of the catalyst. Since volatiles were not recovered in these experiments, their yield was calculated as the difference between the initial mass of PE and that of the recovered soluble and insoluble hydrocarbons.

To characterize volatile reaction products, a mini-autoclave was modified with a gas outlet made from a Swagelok hose connector sealed with a hexagon socket head screw cap containing a Cu O-ring. The total internal volume of the reactor and the gas outlet, 13 mL, was measured by filling with water. The mini-autoclave and a Schlenk flask capped with a white rubber septum were connected via Tygon tubing to a glass vacuum line equipped with a pressure gauge and a port for gas sampling. The Schlenk flask and the lines were evacuated under reduced pressure (10⁻⁴ Torr), then isolated from the pumping system. The gases in the autoclave were expanded into the line by loosening the screw cap, and the pressure was measured. The volatiles yield was estimated using the total volume of the gas line and the Schlenk flask as measured by gas expansion (152 mL), and the ideal gas law. An aliquot of gas (400 μL) was removed via the sampling port for analysis of H₂ by GC-TCD, using a gas-tight syringe. The Ar present in the autoclave at the start of the reaction (1 atm) was used as the internal standard. Propene, which is not a reaction product, was injected into the Schlenk tube as an internal standard for hydrocarbon analysis by GC-FID. An aliquot (0.2 mL) was then removed for analysis. The total GC yields of H₂ and light hydrocarbons are consistent with the total yield of gases based on pressure measurement, with a precision of ±10%.

To analyze volatile hydrocarbons that may have been lost during solvent removal from the chloroform-soluble fraction, the reactor contents were transferred with the aid of chloroform (5 mL) into a Schlenk flask. The contents were distilled at 150° C., while condensing volatile products and chloroform in a receiving flask cooled in a dry ice/acetone bath. The clear, colorless distillate was transferred to a 10 mL volumetric flask, 2 mg mesitylene was added as an internal standard, and the flask was filled to the 10 mL mark with chloroform. The solution was analyzed on an Agilent 6890N Network Gas Chromatograph equipped with a DB-5 column and an FID detector, using response factors measured for toluene and light n-alkanes (C₇-C₁₁) relative to mesitylene. The total mass of recovered volatile products from distillation is small (ca. 1.5 mg, measured by GC-FID), with toluene being the major product.

Some reactions were conducted in a larger autoclave reactor (Parr Series 5000 Multiple Reactor System, internal volume ca. 100 mL, stainless steel). The contents were stirred at 678 rpm, using a magnetic stir bar encapsulated in Pyrex. Timing of the reaction began when the internal temperature reached the set temperature, 250° C.; reported reaction times do not include the time (ca. 30 min) needed to heat the reactor from room temperature. Since the internal thermocouple is located above the solids in the reactor, it reads the temperature of the gas phase rather than the solid/liquid phase. The temperature of the autoclave wall was also measured externally with a thermometer and stabilized at (280±5) ° C. during the reaction time.

The autoclave was cooled to room temperature prior to opening, by immersing the bottom of the vessel in 25° C. water for 15 min. Gases were collected as described above. When the autoclave lid was removed, an oily liquid was observed to have adhered on the lid and separated from the catalyst at the bottom of the vessel. It was recovered by dissolving in hot chloroform, then evaporating the solvent under reduced pressure overnight. The solid products were transferred onto a fine glass frit, then washed with hot chloroform. The solvent was removed from the filtrate by evaporation overnight at 0.1 Torr at room temperature. The insoluble material remaining on the frit, including the catalyst and insoluble hydrocarbons, was recovered and weighed. The mass of insoluble hydrocarbons was calculated by subtracting the initial mass of the catalyst. Some hydrocarbons were not recovered during workup, due to the difficulty in washing the dead volumes in the connectors and valves of the autoclave.

Product Analysis

Gas Chromatography

Hydrocarbons in the gas fraction (C1-C₉) were analyzed quantitatively on a Shimadzu GC-2010 gas chromatograph equipped with a capillary column (Supelco Alumina Sulfate plot, 30 m×0.32 mm) and a flame ionization detector (FID). Propene was added as an internal standard. Relative carbon response factors were assumed to be 1.0 (Dietz, J. Gas Chromatogr. 5, 68-71 (1967); Tong and Karasek, Anal. Chem. 56, 2124-2128 (1984)). The injector and detector temperatures were 200° C. The temperature ramp program was: 90° C. (hold 3 min), ramp 10° C./min to 150° C. (hold 20 min).

Hydrocarbons in the gas fraction (C₁-C₉) were analyzed qualitatively on a Shimadzu GC-2010 gas chromatograph equipped with an Agilent DB-1 capillary column (dimethylpolysiloxane, 30 m×0.25 mm×0.25 μm) coupled to a QP2010 Mass Spectrometer. The injector and detector temperatures were 250° C. The temperature ramp program was: 40° C. (hold 3 min), ramp 25° C. per min to 250° C. (hold 10 min).

H₂ was quantified on a Shimadzu GC-8AIT gas chromatograph equipped with a packed column (ShinCarbon ST 80/100, 2 m×2 mm) and a thermal conductivity detector (TCD), using Ar as the internal standard. The linear response of the TCD signal to the injected volume of H₂ and Ar was confirmed using standard H₂/Ar gas mixtures. The response factors for H₂ (f_{H2}) and Ar (f_{Ar}) were obtained as the slopes of fitted lines. The column, injector and detector temperatures were 130° C. The TCD current: 70 mA and the head pressure were 300 kPa (N₂).

Hydrocarbons in the liquid fraction (C₆-C₃₀) were quantitatively analyzed on an Agilent 6890N Network gas chromatograph equipped with an Agilent DB-5 capillary column (fused silica, 30 m×0.25 mm×0.25 μm) and FID. The inlet and detector temperatures were 300 and 280° C., respectively. The temperature ramp program was: 40° C. (hold 3 min), ramp 25° C./min to 320° C. (hold 10 min). The flow rate was 1.0 mL/min (He) with a split ratio of 5:1.

Gel Permeation Chromatography

Molecular weight distributions of hydrocarbon products were analyzed on an Agilent PL-GPC 220 gel permeation chromatograph, equipped with a PL-Gel Mixed B guard column, three PL-Gel Mixed B columns, and a refractive index (RI) detector. Samples were dissolved in 1,2,4-trichlorobenzene (TCB) containing di-tert-butylhydroxytoluene (BHT, 0.01 wt %), by heating at 150° C. for at least 1 h. Elution was achieved using TCB (with BHT) at 150° C. and 1.0 mL min⁻¹. The molecular weight response was calibrated with monomodal, linear polyethylene standards (Varian). To ensure accurate measurement of low molecular weight materials (M_{w}<1000 g mol⁻¹), PE standards with peak molecular weights Mₚ of 507 and 1180 g mol⁻¹ (Polymer Standards Services) were included in the calibration.

To examine the distribution of chromophores, selected product mixtures were also analyzed by GPC with UV detection. Analysis was conducted on a Waters 2695 HPLC equipped with three Acquity APC XT Columns (45+200+450 pore sizes, 4.6×75 mm) and a guard column (XT, 450 pore size, 4.6×30 mm), a Waters 2410 refractive index (RI) detector and a Waters 2998 photodiode array detector (PDA). Chloroform with 0.25 vol % triethylamine was used as the mobile phase at room temperature and 0.35 mL/min. Calibration was achieved using polystyrene standards (Agilent EasiVial kit, linear response for the range 200<molecular weight<400,000 g/mol).

NMR Spectroscopy

Spectra were recorded in 1,1,2,2-tetrachloroethane-d₂. ¹H NMR spectra were acquired at 600 MHz on a Varian Unity Inova AS600 spectrometer, and were analyzed using MestReNova (v11.0.1, Mestrelab Research S. L.). ¹³C NMR spectra were acquired on a Bruker AVANCE III Ultrashield Plus 800 MHz (18.8 T) spectrometer, and were analyzed using Topspin (v4.0.6, Bruker Biospin). A Bruker TXI HCN cryoprobe was used to enhance sensitivity for direct ¹³C detection. ¹³C spectra were recorded using a long relaxation delay (10 s) to ensure quantitative intensities. Chemical shifts (δ, ppm) were calibrated using the residual proton signals of the solvent and referenced to tetramethylsilane (TMS).

IR Spectroscopy

Spectra were recorded on a Shimadzu Prestige-21 spectrometer. Samples (2 mg) were ground with a mortar and pestle, mixed with KBr (100 mg), and pressed into 15 mm diameter pellets using a force of 4 tons. IR spectra were measured with a resolution of 1.0 cm⁻¹ over the range 400-4000 cm⁻¹. A background spectrum of a KBr pellet was subtracted from each sample spectrum.

Field Desorption Mass Spectroscopy (FD-MS)

Spectra were obtained on a Waters Micromass GCT Premier Time of Flight mass spectrometer, operating with an extraction rod voltage of 12 kV. Samples were dissolved in dichloromethane and loaded directly onto a CARBOTEC FD emitter, consisting of a 10 μm tungsten wire carrying a pyrocarbon coating of microneedles (ca. 120 μm diameter). Nominal mass data were acquired while employing a data-dependent ramp of the emitter current from 0-90 mA, pausing the ramp when more than 30 counts per scan were observed in the base peak ion. The extraction rods were cleaned between acquisitions by slowly raising the extraction rod current to 3 A while maintaining a 12 kV charge. The emitter was cleaned between acquisitions by raising the emitter current to 95 mA for 5 s. The calibration range is 400<m/z<1200, with a maximum mass error of 0.2 m/z. A mixture of triacontane (C₃₀H₆₂), and 1,4-didodecylbenzene (C₃₀H₅₄) in a molar ratio of 1:1.02 was analyzed to assess sensitivity and accuracy for detecting and quantifying different types of hydrocarbons. Each compound gave its molecular ion as the major peak. The similar peak heights confirm that this data is suitable for the semi-quantitative analysis of their relative abundance.

Determination of Selectivity to Alkylaromatics

NMR Method

Although the ¹H and ¹³C NMR spectra are complex due to the presence of multiple isomers, they can be integrated for various functional group regions. The sample calculations below correspond to analysis for the CHCl₃-soluble liquid hydrocarbon fractions recovered from the autoclave after depolymerization of 0.118 g PE (M_{w}=3.52×10³ g mol⁻¹, Ð=1.9) with 0.200 g Pt/γ-Al₂O₃ (1.5 wt % Pt) at 280° C. for 24 h. Results for other product fractions and reaction conditions are summarized in Tables 2A, 2B, and 2C. In Table 2A, standard deviations for selectivities are represented inside the parentheses.

To estimate selectivities for each type of aromatic products, it is assumed that the major aromatic products are dialkylbenzenes and dialkylnaphthalenes. Integration of the aromatic line region gives a ratio of monoaromatic protons to polyaromatic protons, Hₘₒₙₒ/H_{poly}, of 1.6. Thus, the molar ratio of dialkylbenzenes (each with 4H) to dialkylnaphthalenes (each with 6H) is 2.4. Consequently, if dialkylnaphthalenes represent x mol % of all products, then dialkylbenzenes represent 2.4x mol %, and the selectivity to alkylaromatics (S_{Ar}) is the combination of the two fractions, 3.4x mol %. The remainder, (1-3.4x) mol %, corresponds to saturated alkanes (since olefins were not detected in significant amounts by ¹H NMR).

According to GPC analysis, the liquid hydrocarbon products have an Mₙ value of 466 g mol⁻¹ (corresponding to an averaged carbon number C34, in good agreement with the FD-MS result), with a narrow dispersity (Ð=1.12). The aromatic protons (H_{Ar}) in the products are distributed as dialkylnaphthalenes (6x mol %) and dialkylbenzenes (4×2.4x mol %). Furthermore, the total protons (Hₜₒₜₐₗ) are distributed as dialkylnaphthalenes (C₃₄H₅₆, 56x mol %), dialkylbenzenes (C₃₄H₆₂, 62×2.4x mol %), and saturated alkanes (C₃₄H₇₀, 70×(100-3.4x) mol %). The ratio H_{Ar}/Hₜₒₜₐₗ (0.037 observed in ¹³C NMR spectra) is given by eq S1:

$$\frac{{H}_{Ar}}{{H}_{total}} = \frac{6 x + 4 \times 2.4 x}{56 x + 62 \times 2.4 x + 70 ( 100 - 3.4 x )} = 0.037 \qquad ( S1 )$$

The resulting dialkylnaphthalene mole fraction is x=15.4, while the dialkylbenzene mole fraction is 2.4x=36.9. The total selectivity to aromatics in the CHCl₃-soluble hydrocarbons is therefore 52.3 mol %.

A similar assessment was undertaken by integrating the aromatic region (120-150 ppm) and the aliphatic region (10-40 ppm) of the corresponding ¹³C NMR spectrum. The fraction of aromatic carbons is (0.10±0.01). Since carbon atoms associated with naphthalene and benzene rings are difficult to distinguish by ¹³C NMR, the ratio from the ¹H NMR analysis (1:3.5) is used. In addition, the average total carbon number in the hydrocarbon products is C₃₄ according to GPC. Since dialkylnaphthalenes contribute 10x mol % C_{Ar}, while dialkylbenzenes contribute 6×(2.4x) mol % C_{Ar}, the ratio C_{Ar}/Cₜₒₜₐₗ is given by eq S2:

$$\frac{{C}_{Ar}}{{C}_{total}} = \frac{10 x + 6 \times 2.4 x}{34} = 0.1 \qquad ( S2 )$$

The resulting dialkylnaphthalene mole fraction, x, is 0.139, similar to the value obtained by analysis of the ¹H NMR spectrum (0.155). The dialkylbenzene mole fraction is 0.334, for a total aromatic selectivity of 47 mol %. This value is slightly smaller than the value estimated by ¹H NMR (52 mol %), presumably due to experimental uncertainty.

These estimated aromatic selectivities depend on the average carbon number. Due to differences in the hydrodynamic radii of dialkylaromatics compared to linear PE, the experimental values for Mₙ are lower than the actual Mₙ values. For example, a dodecylbenzene standard (246 g mol⁻¹) gave a measured Mₙ of 166 g mol⁻¹, and a di-dodecylbenzene standard (414 g mol⁻¹) gave a measured Mₙ of 345 g mol⁻¹. Therefore, the GPC method underestimates the mass by ca. 17%. Using an average carbon number 16% higher (C₄₀) instead of C34 to assess the alkylaromatic selectivity would result in a slightly higher total aromatic selectivity, 61 mol % by ¹H NMR and 56 mol % by ¹³C NMR. Thus, considering the uncertainty in the average carbon number derived from Mₙ, the overall alkylaromatic selectivity in the liquid fraction is estimated to be (57±5) mol % by ¹H NMR, and (52±4) mol % by ¹³C NMR.

The ¹H NMR analysis described above was applied to the liquid hydrocarbons recovered after 24 h reaction of n-C₃₀H₆₂ in a mini-autoclave reactor at (280±5) ° C., yielding the values for Hₘₒₙₒ/H_{poly} and H_{Ar}/Hₜₒₜₐₗ shown in Table 5. However, due to the lower molecular weights involved, the average carbon number in the liquid products was obtained by GC-FID (instead of GPC). Based on the peak areas and response factors for each species, the product distribution for the liquid products was obtained. The average carbon number (20, on a molar basis) was calculated using on the following formula:

$$average carbon number = {\sum }_{C10}^{C30} mole fraction \times carbon number \qquad ( S3 )$$

The alkylbenzene selectivity was determined by combining this number with the ¹H NMR results.

FD-MS Method

Peaks were separated into several series based on mass and carbon number (n) as shown in Table 1.

TABLE 1
MS Peaks based on mass and carbon number

| | Alter- | | |
|---|---|---|---|
| | nate | Most probable | Alternate |
| m/z | m/z | assignment | assignments |
| 14n | | cycloalkanes | diphenylparaffins, |
| | | | naphthocyclo- |
| | | | paraffins |
| 14n + 2 | 14n − 12 | alkanes, | |
| | | alkylnaphthalenes | |
| 14n + 4 | 14n − 10 | benzodicycloparaffins | |
| 14n + 6 | 14 − 8 | benzocycloparaffins | |
| 14n + 8 | 14n − 6 | alkylbenzenes | |
| 14n + 10 | 14n − 4 | alkylanthracenes/ | tricycloparaffins |
| | | phenanthrenes | |
| 14n + 12 | 14n − 2 | dicycloparaffins | alkylphenalenes, |
| | | | alkylfluorenes |

The yield of paraffins includes alkanes, cycloalkanes, and dicycloparaffins. The yield of monoaromatics includes alkylbenzenes, benzocycloparaffins, and benzodicycloparaffins. In the benzocycloparaffins, it is most likely to be alkyltetralins. The yield of polyaromatics includes alkylnaphthalenes, alkylanthracenes, and alkylphenanthrenes. To estimate selectivities, the heights of the peaks corresponding to each series were summed and divided by the total height for all peaks. The results are shown in FIG. 6 and Table 3. An alternative analysis using the total peak areas of each series yielded similar results.

H₂ Balance

In Exp. 2 (FIG. 3), converting PE (0.064 mmol, M_{w}=1.85×10³ g mol⁻¹, average C₁₃₂) into gas-phase hydrocarbons (C₁₋₉, 0.34 mmol) and liquid-phase molecules (average C₃₀, 0.19 mmol) requires 0.47 mmol H₂, assuming that each H₂ cleaves one C—C bond and generates two alkane fragments (n_{H2}=n_{C1-9}+n_{C30}−n_{C132}. The amount of free H₂ remaining in the gas phase after reaction is 0.11 mmol, for a total of 0.58 mmol H₂. According to ¹H NMR of the liquid products (0.21 mmol, average C₃₀), the mole fractions of alkylbenzene and alkylnaphthalene are 0.33 and 0.19, respectively. The alkylbenzene yield is 0.063 mmol, while the alkylnaphthalene yield is 0.036 mmol. Since each alkylbenzene or alkylnaphthalene molecule generates 4 or 7H₂ molecules, respectively, the total H₂ generated is 0.50 mmol. This value is similar to the value obtained by mass balance (0.58 mmol).

In Exp. S4e (Table 5), converting n-C30H62 (0.23 mmol) gives hydrocarbons ranging from C1 to C29 (0.47 mmol), requiring 0.24 mmol H2. Including the remaining H2 in the reactor (0.064 mmol) gives a total of 0.30 mmol H2. According to 1H NMR, the mole fractions of alkylbenzene and alkylnaphthalene are 0.11 and 0.035, respectively. The alkylbenzene yield is 0.045 mmol, while the alkylnaphthalene yield is 0.011 mmol. The total H2 generated is 0.26 mmol. This value is similar to the value obtained by mass balance (0.30 mmol).

Results

Scheme 1. One-pot synthesis of linear dialkylbenzenes from polyethylene (PE).

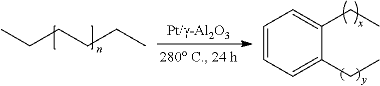

Catalytic Reaction Using Pt/γ-Al₂O₃

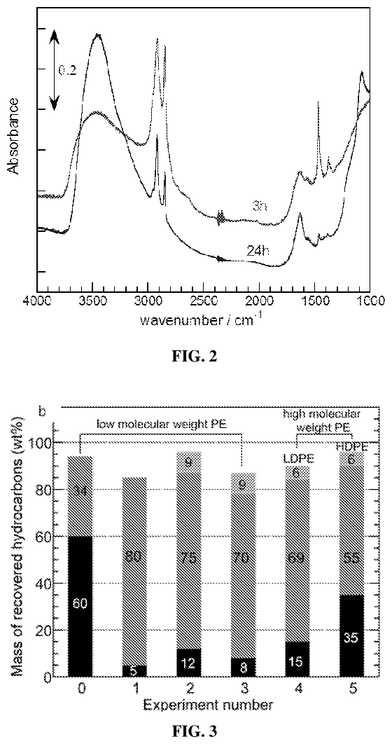

A one-pot, low temperature catalytic method to convert various grades of PE directly to liquid alkylaromatics over a simple heterogeneous catalyst is described. A low molecular weight PE (0.118 g, M_{w}=3.5×10³ g mol⁻¹, Ð=1.90) was combined with Pt/γ-Al₂O₃ (0.200 g, containing 1.5 wt % Pt dispersed as ca. 1 nm nanoparticles, FIG. 1A) in an unstirred mini-autoclave (internal volume 10 mL) without solvent or added H₂. After 24 h at (280±5) ° C., the liquid/wax products (80% by mass) were recovered for characterization by dissolving in hot CHCl₃ (Exp. 1). According to GPC-RI, most of the PE underwent a nearly ten-fold decrease in M_{w}, to 430 g mol⁻¹, as well as the expected decrease in dispersity (to Ð=1.31) (Inaba, et al., Macromolecules 19, 2412-2419 (1986)). Based on their orange color and the appearance of ¹H NMR signals in the region 6.5-9.0 ppm, these hydrocarbons have significant aromatic content. The CHCl₃-insoluble solids include a small amount of organic residue (ca. 5 wt %), in addition to the catalyst. The former includes unreacted polymer and large oligomers (including less soluble alkylaromatics), as judged by IR and ¹H NMR (FIG. 2). The missing mass (ca. 15 wt %) is presumably volatile hydrocarbons and gases, which were not collected in this exploratory experiment. In a control experiment conducted without the catalyst under the same reaction conditions, the PE showed no significant decrease in M_{w}. A second control experiment using the same amount of γ-Al₂O₃ but without Pt resulted mostly in a CHCl₃-insoluble residue (ca. 60 wt %) and a much lower yield of soluble hydrocarbon products (34 wt %), with a smaller decrease in molecular weight (M_{w}=1,421 g mol⁻¹, Ð=1.85) and negligible aromatic content (FIG. 3, Exp. 0).

To obtain a more complete mass balance and to characterize the volatile reaction products, the exploratory experiment was repeated in a mini-autoclave equipped with a gas port. The recovered gases represent a small fraction of the original polymer mass (9 wt %), and include H₂ (0.2 mg) and light hydrocarbons (C₁-C₈, 9.0 mg). The latter are primarily methane, ethane and propane, with minor amounts of n-hexane, cyclohexane, methylcyclopentane, benzene, and n-heptane. Additional volatile hydrocarbons (C₇-C₁₁, 1.5 mg) were recovered by distillation from the autoclave at 150° C. Their major component is toluene (47 wt %). Together, the light hydrocarbons, the CHCl₃-soluble liquids/waxes (89 mg) and the insoluble organic residue (14 mg) represent an overall mass balance of 96% (FIG. 3, Exp. 2).

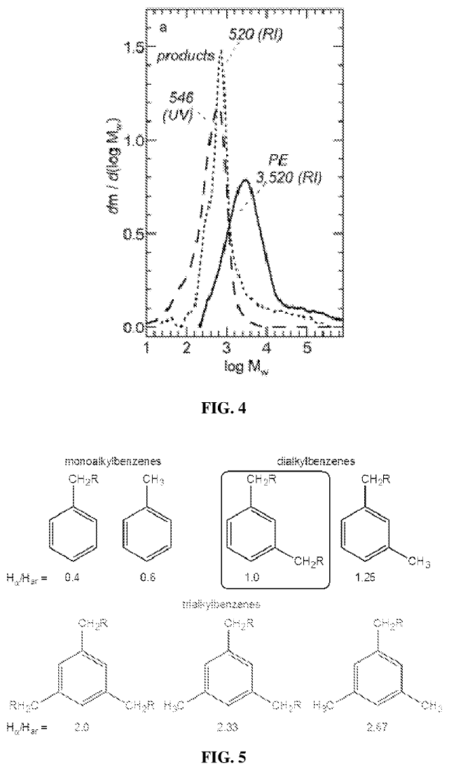

When the reaction was conducted in a larger, stirred autoclave (internal volume 90 mL), most of the PE (70 wt %) was converted at 280° C. to high-boiling liquids/waxes (FIG. 3, Exp. 3). In this case, the waxes (24 wt %, M_{w}=723 g mol⁻¹, Ð=1.34) separated spontaneously from the liquids (46 wt %, M_{w}=520 g mol⁻¹, Ð=1.12) inside the reactor. GPC analysis of the liquid fraction using both RI and UV detection gave similar results (FIG. 4), demonstrating that the UV-active (i.e., aromatic) chromophores are evenly distributed across the molecular weight range. The ¹³C NMR spectrum contains signals in the aromatic region (120-150 ppm), most corresponding to unsubstituted ring carbons. The ¹H NMR spectrum shows that most aromatic protons are associated with benzene rings (6.5-7.4 ppm), with fewer bonded to fused aromatic rings such as naphthalenes (7.4-9.0 ppm) (Behera, et al., in Fluid Catalytic Cracking VII: Materials, Methods and Process Innovations, M. L. Occelli, Ed. (Elsevier, 2007), vol. 166, pp. 163-200). There is no evidence for olefins or dienes (4.5-6.5 ppm).

The process resulted in a high yield of liquid alkylaromatics. Such compounds find widespread application as surfactants, lubricants, refrigeration fluids, and insulating oils (Luo, et al., in Synthetics, mineral oils, and bio-based lubricants: chemistry and technology., L. R. Rudnick, Ed. (CRC press, 2020), pp. 161-180), and their manufacture from waste polyethylene could displace fossil fuel-based routes.

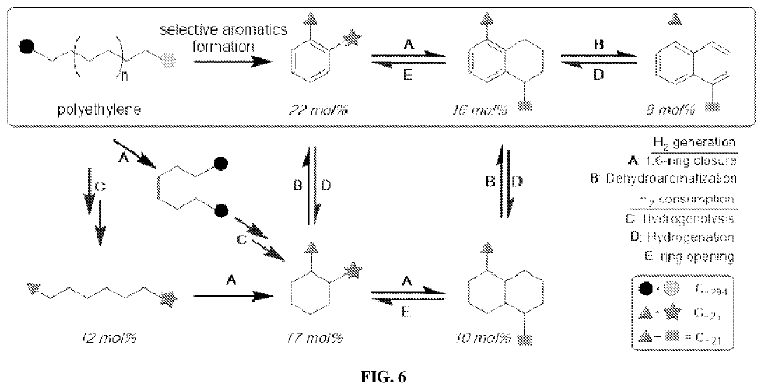

The ¹H NMR spectrum reveals more information about the alkyl substituents. Protons associated with an aliphatic carbon directly bonded to an aromatic ring (C_{α}) resonate in the region 2-4 ppm. The overall ratio H_{α}/H_{aromatic}=1.1 indicates that the major species are, on average, dialkylaromatics (FIG. 5) (Ahuja, et al., Nat. Chem. 3, 167-171 (2011)). A proposed mechanism of formation by dehydrocyclization of polyethylene is provided in FIG. 6. Combining this information with the overall fraction of aromatic protons (0.037), and the average carbon number (C₃₄ for this experiment, based on the Mₙ value determined by GPC), the overall alkylaromatic selectivity in the liquid fraction is estimated to be (57±5) mol %, of which ca. 40 mol % is monoaromatic (Table 2A, Exp. 3).

Using the aromatic carbon fraction (0.10, according to ¹³C NMR) instead results in a similar estimate for the alkylaromatic selectivity, (52±4) mol %. Many of the alkyl substituents are unbranched at the Ca position, judging by the intense ¹H signals at 2.35-2.85 ppm. The paraffinic —CH₂—/—CH₃ ratio, 7.5, demonstrates that each alkyl substituent possesses, on average, <1 branch point.

TABLE 2A
GPC and ¹H NMR characterization of liquid/wax hydrocarbons recovered from
the tandem conversion of various PEs.

| | PE | | Reactor | | | | Products | | | | | | | Total | Total |
|---|---|---|---|---|---|---|---|---|---|---|---|---|---|---|---|
| | Mₙ | | Vol. | Time | Yield | Mₙ | | | H_{Ar}/ | Hₘₒₙₒ/ | S_{Ar} ^{c} | C_{Ar}/ | S_{Ar} ^{d} | Yield | S_{Ar} |
| Exp. | g mol⁻¹ | Ð | mL | h | % | g mol⁻¹ | Ð | C_{avg} ^{b} | Hₜₒₜₐₗ | H_{poly} | mol % | Cₜₒₜₐₗ | mol % | % | mol % |
| 0^{e} | 1.85 × 10³ | 1.90 | 10 | 24 | 34 | 768 | 1.85 | 55 | — | — | — | | | | |
| 1ª | | | 10 | 24 | 80 | 328 | 1.31 | 24 | 0.039 | 0.9 | 40(3) | | | | |
| 2ª | | | 13 | 24 | 75 | 414 | 1.24 | 30 | 0.040 | 1.1 | 52(4) | | | | |
| 3ª | | | 90 | 24 | 46 | 466 | 1.12 | 34 | 0.037 | 1.6 | 57(5) | 0.10 | 52(4) | 70 | 64(5) |
| | | | | | 24 | 538 | 1.34 | 39 | 0.047 | 1.0 | 77(6) | 0.11 | 61(5) | | |
| S1ª | | | 90 | 36 | 42 | 431 | 1.06 | 31 | 0.054 | 2.3 | 75(5) | 0.13 | 64(5) | 55 | 81(6) |
| | | | | | 13 | 551 | 1.06 | 39 | 0.058 | 1.0 | 92(7) | 0.16 | 91(7) | | |
| 4ª | 12.8 × 10³ | 7.37 | 90 | 24 | 39 | 366 | 1.10 | 27 | 0.031 | 1.8 | 39(3) | 0.09 | 37(3) | 69 | 44(4) |
| | | | | | 30 | 355 | 1.12 | 26 | 0.045 | 1.1 | 50(4) | 0.10 | 39(4) | | |
| 5ª | 14.8 × 10³ | 3.61 | 90 | 24 | 20 | 347 | 1.09 | 25 | 0.041 | 1.8 | 46(4) | | | 55 | 50(4) |
| | | | | | 35 | 353 | 1.32 | 26 | 0.047 | 1.0 | 52(4) | | | | |

^{a}Reaction conditions: 118 mg reactant, 200 mg catalyst (Pt/γ-Al₂O₃, 1.5 wt %), 280° C.
^{b} Calculated as Mₙ/14. Where two entries are present for the same experiment, the liquid/wax hydrocarbon products were collected and analyzed separately.
^{c} Selectivity to alkylaromatics (S_{Ar}) calculated based on analysis of ¹H NMR spectrum.
^{d} Selectivity to alkylaromatics (S_{Ar}), as defined above, calculated based on analysis of ¹³C NMR spectrum. Uncertainties are propagated from the uncertainty in the average carbon number.
^{e}Same reaction conditions as a expect for the catalyst, i.e. reaction with γ-Al₂O₃ support only.

TABLE 2B
GPC and ¹H NMR characterization of liquid/wax hydrocarbons
recovered from the conversion of PE under different conditions.

| | | | Products | | | | | | | |
|---|---|---|---|---|---|---|---|---|---|---|
| Exp. | Time h | Temperature ° C. | Liquid Yield wt % | Mₙ | Ð | C_{avg}^{b} | $\frac{{H}_{Ar}}{{H}_{total}}$ % | $\frac{{H}_{monc}}{{H}_{poly}}$ | S_{Ar}^{c} mol % | Insoluble Hydrocarbons wt % |
| S7^{d} | 2 | 330 | 71 | 439 | 2.13 | 31 | 4.2 | 1.8 | 54 | 9 |
| S8ª | 24 | 280 | 31 | 706 | 3.32 | 50 | 1.5 | 2.5 | 33 | 61 |
| S9^{a} | 8 | 360 | 80 | 262 | 1.89 | 19 | 4.4 | 1.7 | 34 | 5 |

^{a}Reaction conditions: 200 mg of PE (Mₙ = 1.85 × 10³ g mol⁻¹, Ð = 1.90), 100 mg reduced catalyst (Pt/γ-Al₂O₃, 1.5 wt % Pt), m_{PE}/m_{Pt} = 133.3, in 13 ml unstirred mini-autoclaves under Ar.
^{b}Calculated as Mₙ /14.
^{c}Selectivity for alkylaromatics (S_{Ar}), as defined above, calculated based on analysis of ¹H NMR spectrum.
^{d}Reaction was conducted using 118 mg of PE (Mₙ = 1.85 × 10³ g mol⁻¹, Ð = 1.90), 200 mg reduced catalyst (Pt/γ-Al₂O₃, 1.5 wt % Pt), m_{PE}/m_{Pt} = 39.3.

TABLE 2C
GPC and ¹H NMR characterization of liquid/wax hydrocarbons
recovered from the conversion of PE under different conditions.

| | | | Products | | | | | | | | |
|---|---|---|---|---|---|---|---|---|---|---|---|
| | | Reactor | Liquid | | | | | | | | Insoluble |
| | Time | Vol. | Yield | Mn | | | | Hₘₒₙₒ/ | S_{Ar}^{c} | Total S_{Ar} | Hydrocarbons |
| Exp. | h | ml | wt % | g mol⁻¹ | Ð | C_{avg}^{b} | H_{Ar}/Hₜₒₜₐₗ | H_{poly} | mol % | mol % | wt % |
| 1ª | 4.0 | 10 | 64 | 560 | 4.56 | 40 | 0.019 | 1.0 | 33 | | 14 |
| 2ª | 12.0 | 10 | 69 | 344 | 4.24 | 25 | 0.029 | 0.7 | 31 | | 8 |
| 3^{a} | 24.0 | 10 | 52 | 203 | 2.38 | 15 | 0.049 | 0.5 | 30 | | 5 |
| 4^{d} | 24.0 | 10 | 52 | 507 | 3.78 | 36 | 0.018 | 0.6 | 29 | | 16 |
| 5^{a} | 12.0 | 90 | 32 | 213 | 2.27 | 15 | 0.123 | 0.4 | 63 | | 14 |
| 6^{a} | 1.0 | 90 | 10 | 227 | 1.38 | 16 | 0.031 | 1.0 | 22 | 29 | 22 |
| | | | 28 | 311 | 6.23 | 22 | 0.033 | 1.0 | 32 | | |
| 7^{e} | 1.0 | 90 | 17 | 271 | 1.36 | 19 | 0.019 | 1.4 | 18 | 29 | 25 |
| | | | 38 | 499 | 4.38 | 36 | 0.023 | 1.2 | 38 | | |
| 8^{f} | 1.0 | 90 | 25 | 233 | 1.39 | 17 | 0.035 | 1.3 | 28 | 41 | 17 |
| | | | 30 | 454 | 4.08 | 32 | 0.044 | 0.9 | 59 | | |

^{a}Reaction conditions: 120 mg PE (Mₙ = 1.85 × 10³ g mol⁻¹, Ð = 1.90), 200 mg Pt/γ-Al₂O₃ (1.5 wt %) and 100 mg F—Al₂O₃ (1.3 wt %), 280° C.
^{b}Calculated as Mₙ/14, where two entries are present for the same experiment, the liquid/wax hydrocarbon products were collected and analyzed separately.
^{c}Selectivity to alkylaromatics (S_{Ar}) calculated based on analysis of ¹H NMR spectrum.
^{d}Reaction conditions: 120 mg PE (Mₙ = 1.85 × 10³ g mol⁻¹, Ð = 1.90), 200 mg γ-Al₂O₃ and 100 mg F—Al₂O₃ (1.3 wt %), 280° C.
^{e}Reaction conditions: 120 mg PE (Mₙ = 1.85 × 10³ g mol⁻¹, Ð = 1.90), 200 mg Pt/γ-Al₂O₃ and 50 mg F—Al₂O₃ (1.3 wt %), 280° C.
^{f}Reaction conditions: 120 mg PE (Mₙ = 1.85 × 10³ g mol⁻¹, Ð = 1.90), 200 mg Pt/γ-Al₂O₃ and 100 mg F—Al₂O₃ (0.7 wt %), 280° C.

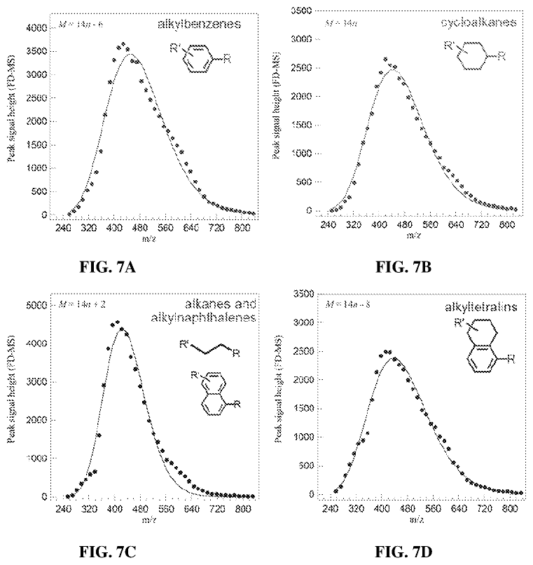

Individual molecular components in the liquid fraction were identified using field desorption-mass spectrometry (FD-MS). Each mass series shows a log-normal distribution with a maximum intensity at ca. C₃₀ (FIG. 7). The most abundant products are the alkylbenzene series (14n-6, ca. 22 mol %). Saturated alkanes and alkylnaphthalenes share the same mass profile (14n+2, 20 mol %) and are the next most abundant group, with smaller amounts of alkyltetralins (14n-8, 16 mol %) and alkylnaphthenes (i.e., alkylcycloalkanes, 14n, 17 mol %). Alkylnaphthalenes presumably arise by further dehydrocyclization of alkylbenzenes (FIG. 6) (Mostad, et al., Appl. Catal. 63, 345-364 (1990)). Minor aromatic products include polyaromatics, such as alkyl-anthracenes and -phenanthrenes (14n-4, 7 mol %) and their partially hydrogenated analogs (14n-10, 8 mol %).

According to FD-MS, the selectivity for mono-aromatic products (including both alkylbenzenes and alkyltetralins) is ca. 40 mol %, consistent with the ¹H NMR analysis described above.

The alkylnaphthene products, which have intrinsic value as solvents and hydrogen donors (Zhu, et al., Energ. Environ. Sci. 8, 478-512 (2015)), could be further dehydrogenated to alkylaromatics by active control of P(H₂) in the reactor. The total yield of cyclic products (both alkylaromatics and alkylnaphthenes) in the liquid products is 88 mol % (Table 3).

TABLE 3
FD-MS selectivities for various mass series in the liquid hydrocarbons recovered from tandem PE conversion^{a}

| | | Method 1 ^{b} | | | Method 2 ^{c} | | |
|---|---|---|---|---|---|---|---|
| Mass series | Major assignment | area | | % | area | | % |
| 14n | cycloparaffins | 63270 | | 17 | 5282 | | 19 |
| 14n + 2 | alkanes, alkylnaphthalenes | 77706 | | 20 | 5914 | | 21 |
| 14n + 4 | benzodicycloparaffins | 31089 | | 8 | 2200 | | 8 |
| 14n + 6 | benzocycloparaffins | 60489 | | 16 | 3714 | | 14 |
| 14n + 8 | alkylbenzenes | 85390 | | 22 | 5229 | | 19 |
| 14n + 10 | alkylanthracenes, etc. | 27776 | | 7 | 2117 | | 8 |
| 14n + 12 | dicycloparaffins | 37993 | | 10 | 3047 | | 11 |
| Total | | 383713 | | 100 | 27504 | | 100 |
| monoaromatics | | | 22 + 16 + 8 = | 46 | | 19 + 14 + 8 = | 41 |
| polyaromatics^{d} | | | 7 + 8 (of 20) = | 15 | | 8 + 6 (of 21) = | 14 |
| paraffins | | | 17 + 10 + 12 (of 20) = | 39 | | 19 + 11 + 15 (of 21) = | 45 |

^{a}Exp. 3.
^{b} Based on peak heights.
^{c} Based on peak areas.
^{d}The ratio ¹Hₘₒₙₒ/¹H_{poly} = 1.6 was used to calculate the fractional contribution of alkylnaphthalenes in the (14n + 2) mass species.

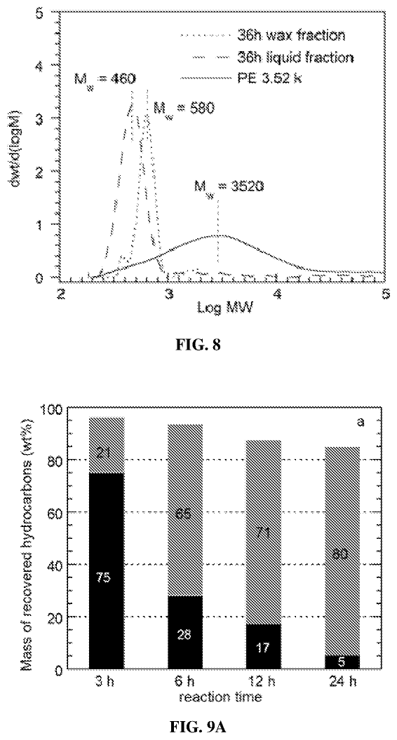

When the reaction time was extended from 24 to 36 h at 280° C., similar products were formed (Table 2A, Exp. S1), although the molecular weight distributions of both liquid and wax fractions shifted to slightly lower values (FIG. 8) and the dispersity decreased further (to Ð=1.06). At the same time, the alkylaromatic selectivity increased (24 h: 52 and 71 mol %; 36 h: 70 and 88 mol %, in the liquid and wax fractions, respectively), Table 2A.

Alkylaromatic yields are also strongly temperature-dependent. A low molecular weight PE (0.118 g, M_{w}=3.5×10³ g mol⁻¹, Ð=1.90) was combined with Pt/γ-Al₂O₃ (0.200 g, containing 1.5 wt % Pt dispersed as ca. 1 nm nanoparticles, FIG. 1A) in an unstirred mini-autoclave (internal volume 10 mL) without solvent or added H₂. After 24 h at a lower temperature (250° C.), the CHCl₃-soluble hydrocarbons (13 wt %) showed a smaller extent of depolymerization (M_{w}=1.8×10³ g mol⁻¹, Ð=2.11) and negligible aromatic content; most PE was simply not converted. At a higher reaction temperature (330° C.), the polymer was largely converted in 24 h, however, the major products (77 wt %) were gases and volatile hydrocarbons. The yield of CHCl₃-soluble hydrocarbons was low (ca. 10 wt %), although the overall yield of aromatics was higher (H_{aromatic}/Hₜₒₜₐₗ=0.38), with more polyaromatics (H_{monoaromatic}/H_{polyaromatic}=0.25). However, after only 2 hours at 330° C. (Table 2B, Exp. S7), 71 wt % of polymer was converted into liquid hydrocarbons (Mₙ=439 g mol⁻¹, Ð=2.13, C_{avg}=31) with alkylaromatic selectivity of 54 mol % (39 mol % is monoaromatics). The yield, average carbon number, and alkylaromatic selectivity of the liquid products obtained at 330° C. after 2 hours are similar to the products obtained at 280° C., which require a much longer reaction time of 24 hours (Table 2A, Exp. 1,2). These results at 330° C. may be due to faster hydrogenolysis and aromatization reactions at the higher temperature, i.e. 330° C., compared to the reaction at 280° C.

When the polymer to catalyst ratio increases from m_{PE}/m_{Pt}=39.3 (0.118 g of PE, 0.200 g of 1.5 wt % Pt/alumina) to m_{PE}/m_{Pt}=133.3 (0.200 g of PE, 0.100 g of 1.5 wt % Pt/alumina), the reaction at 280° C. for 24 hours (Table 2B, Exp. S8) converted 31 wt % of PE to wax with a limited decrease in molecular weight (Mₙ=706 g mol⁻¹, Ð=3.32, C_{avg}=50) and a small aromatic content (H_{aromatic}/Hₜₒₜₐₗ=0.015). However, the reaction at 360° C. for 8 hours (Table 2B, Exp. S9) converted 80 wt % of PE to liquid with a much lower molecular weight (Mₙ=262 g mol⁻¹, Ð=1.89, C_{avg}=19) and a higher aromatic content (H_{aromatic}/Hₜₒₜₐₗ=0.042). These results indicate that a reasonably higher temperature can significantly increase the rate of the tandem hydrogenolysis/aromatization of PE to the desired products. The temperature for alkylbenzene formation from low molecular weight PE (0.118 g, Mw=3.5×10³ g mol⁻¹, Ð=1.90 in a reactor containing Pt/γ-Al₂O₃ as the catalyst support is 250° C.≥T≤360° C.

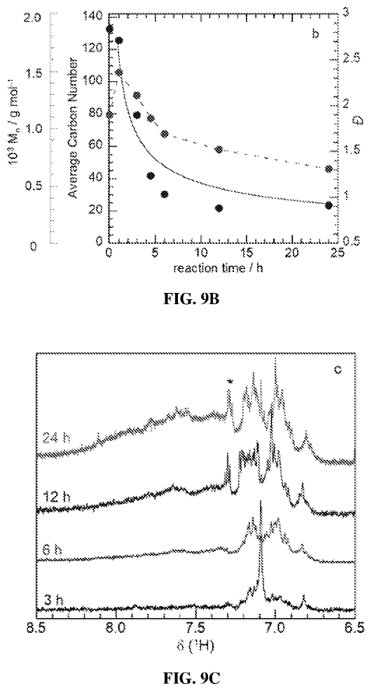

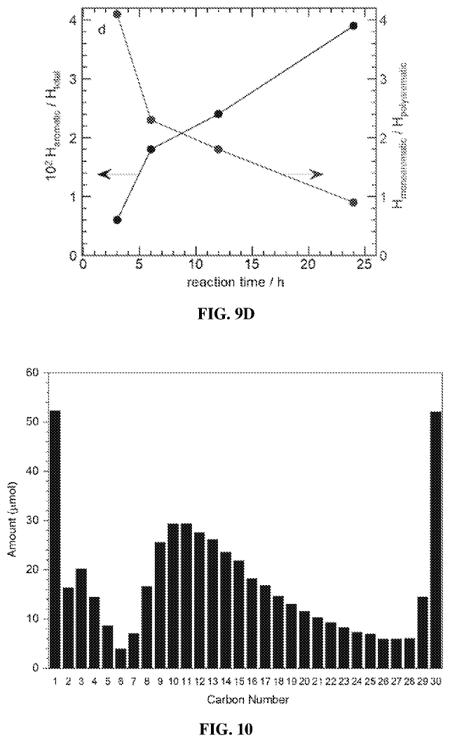

The time course of PE depolymerization was studied at 280° C. (Table 4, Exp. 1a-f). A short induction period, lasting about 1 h, corresponds in large part to the thermal equilibration of the reactor (ca. 45 min). After this time, the liquid hydrocarbon fraction increased (FIG. 9A) as Mₙ decreased, eventually approaching a plateau at 315 g mol⁻¹ after about 6 h (FIG. 9B). The dispersity D increased initially from 1.94 to 2.36, then decreased to stabilize at 1.31. The alkylaromatic yield also changed significantly over the course of the reaction. After 3 h, aromatic protons represented <1% of all protons, mainly associated with alkylbenzenes (FIGS. 9C-9D). At longer reaction times, the aromatic fraction and the yield of alkylnaphthalenes increased (Table 4).

Catalytic Reaction Using Pt/Silica-Alumina

As noted above, when a low molecular weight PE (0.118 g, M_{w}=3.5×10³ g mol⁻¹, Ð=1.90) was combined with Pt/γ-Al₂O₃ (0.200 g, containing 1.5 wt % Pt dispersed as ca. 1 nm nanoparticles, FIG. 1A) in an unstirred mini-autoclave (internal volume 10 mL) without solvent or added H₂, after 24 h at a lower temperature (250° C.), the CHCl₃-soluble hydrocarbons (13 wt %) showed a smaller extent of depolymerization (M_{w}=1.8×10³ g mol⁻¹, Ð=2.11) and negligible aromatic content; most PE was simply not converted.

When Pt/silica-alumina (0.200 g, containing ˜1 wt % Pt, with metal dispersion of 43%) was applied as catalyst, the reaction at 250° C. after 24 hours converted 76 wt % of PE (0.118 g, M_{w}=3.5×10³ g mol⁻¹, Ð=1.90) into wax (Mₙ=415 g mol⁻¹, Ð=6.18, C_{avg}=30), with aromatics content of H_{aromatic}/Hₜₒₜₐₗ=0.033 and an alkylaromatic selectivity of 35 mol % (10 mol % is monoaromatics, i.e 10 mo % alkylbenzene and 25 mol % of alkylnaphthalene). The depolymerization using Pt/silica-alumina catalyst produced alkylnaphthalene, which is a valuable alkylaromatic that can be used as intermediates for producing advanced polymers (e.g. short-chain alkylnaphthalene) and in the synthesis of alkylated naphthalene sulfonic acids and as lubricating base oils (e.g. short-chain alkylnaphthalenes) (Li, et al., Chemistry Select, 4:5284-5290 (2019)).

Catalytic Reaction Using a Mixture of Pt/γ-Al₂O₃ and F-γ-Al₂O₃

A low molecular weight PE (0.120 g, M_{w}=3.5×10³ g mol⁻¹, Ð=1.90) was combined with Pt/γ-Al₂O₃ (0.200 g, containing 1.5 wt % Pt dispersed as ca. 1 nm nanoparticles, FIG. 1A) and F-γ-Al₂O₃ (0.100 g, 1.3 wt % fluorine on γ-Al₂O₃) in an unstirred mini-autoclave (internal volume 10 mL) without solvent or added H₂. The reaction using mixed catalysts after 4 hours showed a comparable yield of liquid hydrocarbons (64 wt %) and with a higher alkylaromatic selectivity (33 mol %, of which 20 mol % is monoaromatics) to the reaction using only Pt/γ-Al₂O₃ as catalyst after 6 hours (alkylaromatic selectivity of 26 mol %, of which 19 mol % is monoaromatics) (Table 4, Exp. 1e). Although liquid hydrocarbons obtained from the reaction with mixed catalysts after 12 hours (Table 2C, Exp. 2) did not show significant decrease in M_{w} (1459 g mol⁻¹) and had a relatively high dispersity (Ð=4.24) compared with the reaction using only Pt/γ-Al₂O₃ (Table 4, Exp. If), the reaction after 24 hours (Table 2C, Exp. 3) resulted in a further decrease in Mₙ, to 203 g mol⁻¹ (C_{avg}=15), in comparison to Mₙ of 328 g mol⁻¹ (C_{avg}=23) from the reaction without the addition of F-γ-Al₂O₃(Table 4, Exp. 1g). The overall yield of aromatics was also higher with F-γ-Al₂O₃ (H_{aromatic}/Hₜₒₜₐₗ=0.049). In a control experiment conducted with only F-γ-Al₂O₃ for 24 hours (Table 2C, Exp. 4), a limited decrease in molecular weight (M_{w}=1916 g mol⁻¹, Ð=3.78) was observed.

To avoid heat and mass transfer limitation, the reaction was conducted in a larger, stirred autoclave (internal volume 90 mL). As a result, the product distribution shifted toward lower molecular weight products, where 40 wt % of PE was converted to gaseous products, along with a lower yield of liquid hydrocarbons (32 wt %), with a much higher aromatic content (H_{aromatic}/Hₜₒₜₐₗ=0.123) and alkylaromatic selectivity (63 mol %, of which 21 mol % is monoaromatics) compared to the products obtained from the reaction after 12 hours in an unstirred mini-autoclave (Table 2C, Exp. 2 and 5).

As depolymerization of PE was accelerated with the addition of F-γ-Al₂O₃, less time was required to achieve high yield of desired products. While the reaction catalyzed by 0.200 g Pt/γ-Al₂O₃ showed no apparent reactivity in 1 hour (Mₙ=1758 g mol⁻¹, C_{avg}=126), the reaction using a physical mixture of 0.200 g Pt/γ-Al₂O₃ and 0.100 g F-γ-Al₂O₃(Table 2C, Exp. 6) converted PE to gaseous (27 wt %) and liquid hydrocarbons (38 wt %) with an estimated alkylaromatic selectivity of 22 mol % in liquid hydrocarbons and 32 mol % in wax hydrocarbons. The gaseous products were composed of mostly propane, butane and pentane, which have higher values than methane and ethane that were produced as the major products in PE reaction using Pt/γ-Al₂O₃(FIG. 3, Exp. 2). In a larger, stirred autoclave, the waxes (28 wt %, M_{w}=311 g mol⁻¹, C_{avg}=22) separated spontaneously from the liquids (10 wt %, Mₙ=227 g mol⁻¹, C_{avg}=16) inside the reactor. In this case, the average carbon number obtained from both liquid and wax after 1 hour was lower than those obtained from the reaction without F-γ-Al₂O₃ after 24 hours (C_{avg}=34 for liquid and C_{avg}=39 for wax, Table 4, Exp. 1g).

To minimize the formation of gases and increase the yield of liquid hydrocarbons, the reaction was conducted with a lower number of acid sites (0.050 g 1.4 wt % F-γ-Al₂O₃) (Table 2C, Exp. 7) compared to the reaction using 0.100 g of 1.3 wt % F—Al₂O₃(Table 2C, Exp. 6). By lowering the number of acid sites, the yield of liquid hydrocarbons increased by 17 wt %; however, with a higher alkylaromatic selectivity (38 mol %) in wax hydrocarbons; however, alkylaromatic selectivity decreased by 4 mol % in liquid hydrocarbons. Another experiment using 0.100 g of 0.7 wt % F-γ-Al₂O₃ increased the alkylaromatic selectivity in both liquid (28 mol %) and wax (59 mol %) hydrocarbon products (Table 2C, Exp. 8).

TABLE 4
GPC and ¹H NMR characterization of liquid/wax hydrocarbons recovered from tandem
PE conversion ª as a function of reaction time (Exp. 1a-g).

| | | Products | | | | | | |
|---|---|---|---|---|---|---|---|---|
| | Time | Yield | M_{w} | Mₙ | | | | Hₘₒₙₒ/ |
| Exp. | h | % | g mol⁻¹ | g mol⁻¹ | Ð | C_{avg} ^{b} | H_{Ar}/Hₜₒₜₐₗ | H_{poly} |
| 1a | 1.0 ^{c} | — | 4156 | 1758 | 2.36 | 126 | — | — |
| 1b | 3.0 ^{c} | — | 2350 | 1110 | 2.11 | 79 | — | — |
| 1c | 3.0 | 21 | 1177 | 789 | 1.49 | 56 | 0.004 | 3.8 |
| 1d | 4.5 ^{c} | — | 1084 | 584 | 1.86 | 42 | — | — |
| le | 6.0 | 66 | 716 | 424 | 1.69 | 30 | 0.018 | 2.0 |
| 1f | 12.0 | 70 | 460 | 302 | 1.52 | 22 | 0.024 | 1.8 |
| 1g ^{d} | 24.0 | 80 | 430 | 328 | 1.31 | 23 | 0.039 | 0.9 |

^{a} Reaction conditions: 118 mg PE (Mₙ = 1.85 × 10³ g mol⁻¹, Ð = 1.90), 200 mg catalyst (Pt/γ-Al₂O₃, 1.5 wt % Pt), 280° C., 10 mL mini-autoclave. Each experiment was performed independently (not as continuous sampling from one experiment).
^{b} Calculated as Mₙ/14.
^{c} GPC analysis was performed on the crude reaction mixture.
^{d} This experiment is the same as Exp. 1 in FIG. 3 and Table 2A.

### Example 2. The Overall Depolymerization Reaction of Polyethylene is Thermodynamically Favorable

Materials and Methods

Depolymerization of polyethylene is performed as described above.

Catalytic conversion of n-C₃₀H₆₂ is performed as follows.

A mixture of 0.120 g n-C₃₀H₆₂ and 0.200 g Pt/γ-Al₂O₃ was heated at (280±5) ° C. under Ar for 24 h in mini-autoclaves. After the reaction, the gas products were analyzed by GC-TCD and GC-FID as described above. The remaining material was extracted with CHCl₃ at room temperature, separated from the insoluble catalyst as a yellow liquid, then, mesitylene (2.0 mg) was added as an internal standard. The solution was analyzed on an Agilent 6890N gas chromatograph equipped with a DB-5 column and an FID detector.

Peak assignments were made by comparing retention times to a standard mixture of n-alkanes (C₇-C₄₀), standard mesitylene, as well as by matching to peaks identified by GC-MS with the aid of a mass spectral library (NIST 107). Among the liquid products, the major species are n-alkanes (C₇-C₃₀). The minor products are mostly branched alkanes, with smaller contributions from cycloalkanes and alkylbenzenes. The presence of multiple isomers with overlapping peaks hindered more complete analysis. Nevertheless, hydrocarbons that elute together have the same carbon number and very similar response factors (within 3%) (Dietz, J. Gas Chromatogr. 5, 68-71 (1967); Tong and Karasek, Anal. Chem. 56, 2124-2128 (1984)). Therefore, based on the mass of mesitylene, the peak areas and response factors for each species, the absolute amount of each species could be obtained. Since there is a negligible amount of insoluble hydrocarbon in the solid phase (less than 0.1% by mass), the species detected in both gas and liquid phases (89% yield) were combined to obtain a total distribution (FIG. 10). C₅, C₆, and C₇ compounds were not fully accounted for because their signals overlapped with that of CHCl₃.

For ¹H NMR analysis, undeuterated CHCl₃ was removed by evaporation overnight at 0.1 Torr, resulting in a ca. 70 wt % yield of liquid products consisting only of molecules larger than C₉. Mesitylene was also mostly removed (<0.1 mol % remaining) as evidenced by GC-FID analysis.

Thermodynamic Analysis of Tandem Hydrogenolysis/Aromatization

The thermodynamic feasibility of the tandem reaction was assessed using data computed using Benson group increment theory for gas phase alkanes and alkylbenzenes (Stein and Brown, NIST Chemistry WebBook, NIST Standard Reference Database Number 69, P. J. Linstrom, W. G. Mallard, Eds. National Institute of Standards and Technology, Gaithersburg Md., 20899, doi.org/10.18434/T4D303). Increment values are compiled in Table 6. The thermodynamic contributions to a linear alkane CₙH₂ₙ₊₂ are:

g_{A,n}=(n−2)g₁+2g₂ (S4)

The thermodynamic contributions for the alkane hydrogenolysis reaction (eq S5), i.e. g_{Hy}, are given in eq S6:

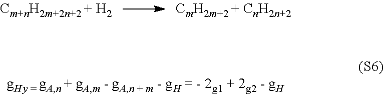

Note that the linearity of the Benson group contributions eliminates the chain length dependence according to eq S8, since changes in the number of methylene carbons (−2g₁) and the number of methyl chain ends (+2g₂) are independent of the initial chain length.

The thermodynamic contributions to an ortho-dialkylbenzene CₙH₂ₙ₋₆, i.e. g_{B,n}, are given in eq S7:

g_{B,n}=(n−10)g₁+2g₂+2g₃+4g₄+2g₅+g₆ (S7)

The thermodynamic contributions to the aromatization reaction (eq S8), i.e. g_{Ar}, are given in eq S9:

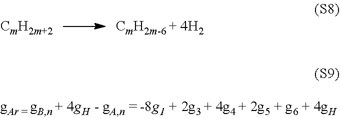

Note that the linearity of the Benson group contributions also removes the chain length dependence from eq S9, since the change in the number of methylene carbons (−8g₁) is independent of the initial chain length.

Derivation of Rate Model for Tandem Hydrogenolysis/Aromatization

In the simplified kinetic model, it is assumed that the hydrocarbon products contain only two types of carbon: aliphatic, and aromatic. Furthermore, the kinetic model is comprised of only two reactions, hydrogenolysis (eq S5) and aromatization (eq S8). Once an aromatic ring is formed, it is assumed it does not undergo further reaction. However, the aliphatic substituents on an aromatic ring can continue to undergo hydrogenolysis and aromatization.

Since there is no solvent in the reactor, each surface Pt atom is in contact with either an aliphatic carbon (i.e., methyl or methylene) A, or an aromatic carbon B. It is assumed that the two types of carbon compete for adsorption sites according to eq S10, where A_{ads} and B_{ads} are adsorbed aliphatic and aromatic carbons, respectively.

A_{ads}+B [symbol] A=B_{cds} (S10)

Adsorption is governed by the equilibrium expression, eq S11, where θ_{A} and θ_{B} are the surface coverages of A and B, respectively:

$$K = \frac{[ A ] {θ}_{B}}{[ B ] {θ}_{A}} \qquad ( S11 )$$

The site balance and mass balance require:

θ_{A}+θ_{B}=1 (S12)

n_{A}+n_{B}=n_{C} (S13)

where n_{A} and n_{B} are the number of aliphatic and aromatic carbons at any time, and n_{C} is the total number of carbons in the system. If the amounts of adsorbed A and B are small relative to the total amounts of A and B (true except in the earliest stages of reaction, when n_{B} is very small), then the equilibrium expression for θ_{A} is solved as follows:

$$\frac{[ A ]}{[ B ]} = \frac{{n}_{A}}{{n}_{B}} \qquad ( S 14 )$$

$${θ}_{A} = \frac{{n}_{A}}{{n}_{A} + K ( {n}_{C} - {n}_{A} )} \qquad ( S 15 )$$

Since one aromatization reaction (eq S8) forms 6 aromatic carbons and removes 6 aliphatic carbons, the rate of aromatization is:

$${r}_{Ar} = \frac{1}{6} \frac{{dn}_{B}}{dt} = - \frac{1}{6} \frac{{dn}_{A}}{dt} \qquad ( S 16 )$$

The rate of aromatization, relative to the rate of hydrogenolysis, is constrained by two factors: the observed mass balance on hydrogen and the observed aromatic product fraction. The model cannot simultaneously satisfy both constraints, as the mass balance on hydrogen is distorted by contributions of significant non-aromatic, nonlinear molecules, and the aromatic fraction is distorted by the presence of large amounts of light hydrocarbon gases. These gases represent a large portion of the scission products on a molar basis, even if they account for a relatively small portion of the mass in the system. To avoid introducing additional complexity to the model, the light hydrocarbons and hydrogen balance is ignored, focusing solely on the liquid products.

The experimental ¹H NMR, ¹³C NMR and GPC results indicate an overall selectivity for aromatic molecules in the liquid product of ca. 50%. The number of molecules consisting only of aliphatic carbons is denoted as N_{A}, and the number of molecules with at least one aromatic carbon is denoted as N_{B}. Each hydrogenolysis (eq S5) reaction results in a net increase of one solely aliphatic molecule, and each aromatization (eq S8) reaction converts one aliphatic molecule to one aromatic molecule. Assuming the number of initial molecules is small and the reaction rates are approximately constant over a short period of time, the number of molecules of each time evolve with time t as

N_{A}=r_{Hy}t−r_{Ar}t (S17)

N_{B}=r_{Ar}t (S18)

The molar selectivity among the products s can be written as

$$\frac{1}{2} = s = \frac{{N}_{B}}{{N}_{A} + {N}_{B}} = \frac{{r}_{Ar}}{{r}_{Hy}} \qquad ( S 19 )$$

This shows that the rate of aromatization (eq S8) is half that of the rate of hydrogenolysis (eq S5). Therefore, the rate of hydrogenolysis can be written in terms of the rates of formation of either aromatic or aliphatic carbons:

$${r}_{Hy} = \frac{1}{s} {r}_{Ar} = \frac{1}{6 s} \frac{{dn}_{B}}{dt} = - \frac{1}{6 s} \frac{{dn}_{A}}{dt} \qquad ( S 20 )$$

The rate law for hydrogenolysis is assumed to follow first-order in both the mass of catalytic Pt, m_{Pt}, and the coverage of active Pt sites by aliphatic carbons, OA, with a second-order rate constant k (dimensions g_{Pt}⁻¹ s⁻¹):

$${r}_{Hy} = {kn}_{Pt} {θ}_{A} = \frac{{km}_{Pt} {n}_{A}}{{n}_{A} + K ( {n}_{C} - {n}_{A} )} \qquad ( S 21 )$$

$$\frac{{dn}_{A}}{dt} = \frac{- 6 {skm}_{Pt} {n}_{A}}{{n}_{A} + K ( {n}_{C} - {n}_{A} )} \qquad ( S 22 )$$

Integration of eq S22 leads to the following expressions:

$${\int }_{{n}_{A , 0} = {n}_{C}}^{{n}_{A}} \frac{{n}_{A} + K ( {n}_{C} - {n}_{A} )}{{n}_{A}} {dn}_{A} = - 6 {skm}_{Pt} {\int }_{0}^{t} dt \qquad ( S 23 )$$

$${Kn}_{C} ln \frac{{n}_{A}}{{n}_{C}} + ( K - 1 ) ( {n}_{C} - {n}_{A} ) = - 6 {skm}_{Pt} t \qquad ( S 24 )$$

For K>>1, the integrated rate law simplifies to:

$$ln \frac{{n}_{A}}{{n}_{C}} + ( 1 - \frac{{n}_{A}}{{n}_{C}} ) = \frac{- 6 {skm}_{Pt} t}{{Kn}_{C}} ≡ - τ \qquad ( S 25 )$$

where τ is a dimensionless time. This equation can be solved for the fraction of aliphatic carbon, n_{A}/n_{C}, using the Lambert W function:

$$\frac{{n}_{A}}{{n}_{C}} = - W ( {e}^{- ( τ + 1 )} ) \qquad ( S 26 )$$

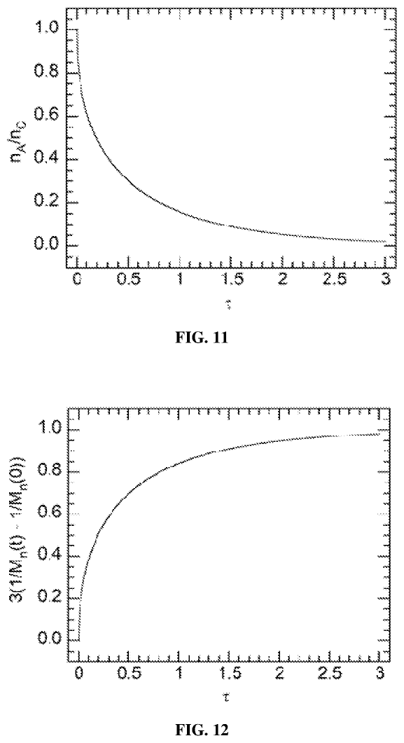

The shape of this function is shown in FIG. 11.

Eq S27 can predict the evolution of the number-average chain length of all chains, Mₙ. The loss of mass to light hydrocarbons is assumed to be negligible. First, note that the number of aromatic carbons is given by n_{B}=n_{C}−n_{A}, and that six aromatic carbons correspond to one aromatization event. The number of hydrogenolysis events is n_{B}/(6s), or n_{B}/3 for s=1/2. Each hydrogenolysis event results in a new chain, therefore the total number of chains present at time t, Nₜ, is:

$${N}_{t} = {N}_{0} + \frac{1}{6 s} {n}_{B} = {N}_{0} + \frac{1}{6 s} ( {n}_{C} - {n}_{A} ) \qquad ( S 27 )$$

where N₀ is the initial number of polymer chains in the system. The number-average chain length, Mₙ, evolves in time as:

$${M}_{n} ( t ) = \frac{{n}_{C}}{{N}_{t}} = \frac{{n}_{C}}{{N}_{0} + \frac{1}{6 s} ( {n}_{C} - {n}_{A} )} \qquad ( S 28 )$$

$$Thus$$

$$\frac{1}{{M}_{n} ( t )} = \frac{{N}_{0}}{{n}_{C}} + \frac{{n}_{C} - {n}_{A}}{6 {sn}_{C}} = \frac{1}{{M}_{n} ( 0 )} + \frac{1}{6 s} ( 1 - \frac{{n}_{A}}{{n}_{C}} ) \qquad ( S 29 )$$

Rearranging eq S29 for n_{A}/n_{C} and substituting into eq S25 yields the relationship between M₁ and reaction time:

$$1 - \frac{{n}_{A}}{{n}_{C}} = \frac{6 s}{{M}_{n} ( t )} - \frac{6 s}{{M}_{n} ( 0 )} \qquad ( S 30 )$$

$$ln ( 1 - \frac{6 s}{{M}_{n} ( t )} + \frac{6 s}{{M}_{n} ( 0 )} ) + ( \frac{6 s}{{M}_{n} ( t )} - \frac{6 s}{{M}_{n} ( 0 )} ) = - τ \qquad ( S 31 )$$

Plotting the inverse number average molecular weight in terms of dimensionless time yields FIG. 11 inverted in the y-axis, shown in FIG. 12.

The example above pertains to a system with no aromatic carbons initially present, n_{B}(0)=0. If this is not the case, the algebra becomes more complicated, but an expression for the evolution of the average chain length may still be found. As aromatic carbons occupy catalytic sites without participating in hydrogenolysis, their presence slows down hydrogenolysis. When n_{B}(0) is large relative to m_{Pt}, no aliphatic carbons adsorb, and no hydrogenolysis takes place.

An additional effect not included in the model is the ability of aromatics to consume hydrogen for their own hydrogenation. This may become significant at sufficiently high aromatic concentrations.

Finally, a model is considered in which aromatization (eq S8) does not occur. This is the case when the H₂ partial pressure is very large, and the reaction equilibrium disfavors further H₂ production via aromatization. Thus, assuming no initial aromatics (n_{B}(0)=0) and no aromatization (r_{Ar}=0), the rate of formation of new aliphatic chains is written as:

$$\frac{dN}{dt} = {r}_{Hy} = {km}_{Pt} \qquad ( S 32 )$$

Integration of eq S32 leads to

Nₜ=N₀+km_{Pt}t (S33)

Analogous to eq S28, the number-average chain length evolves in time as:

$${M}_{n} ( t ) = \frac{{n}_{C}}{{N}_{t}} = \frac{{n}_{C}}{{N}_{0} + {km}_{Pt} t} \qquad ( S 34 )$$

Rearranging and noting that n_{C}/N₀=Mₙ(0) yields an expression similar to eq S31:

$$\frac{1}{{M}_{n} ( t )} - \frac{1}{{M}_{n} ( 0 )} = \frac{{km}_{Pt}}{{n}_{C}} ≡ {τ}_{H} \qquad ( S 35 )$$

where τ_{H} is an alternative dimensionless time for the case of hydrogenolysis without aromatization.

Both models (i.e., with and without the contribution of the aromatization reaction) display similar behaviors. In both, the evolution in average chain length slows and appears to halt, due to the constant rate of new chain production. Since the number of chains increases at a constant pace, chains made later in the reaction have a smaller impact on the average chain length. When aromatization is possible, the kinetics of chain cleavage are slowed even more, because the active sites become progressively occupied by strongly adsorbed aromatics. This has a more severe effect on the leveling of the average chain length, relative to the model without aromatic formation.

Results

The thermodynamics of n-alkane aromatization was assessed. The temperature needed to achieve appreciable aromatic yields for this endothermic reaction (eq 1) decreases as the molecular weight increases (Pradhan, et al., Chem. Sci. 3, 2958-2964 (2012)).

CₘH₂ₘ₊₂→CₘH₂ₘ₋₆+4H₂ (1)

Nevertheless, direct PE conversion to aromatics involves very mild conditions, relative to the much higher operating temperatures generally used for making BTX from molecular n-alkanes (Scheme 1). Thermodynamic values for converting linear PE chains to alkylaromatics at 280° C. in 1 atm H₂, estimated using Benson group contributions for long-chain n-alkanes (Stein and Brown, NIST Chemistry WebBook, NIST Standard Reference Database Number 69, P. J. Linstrom, W. G. Mallard, Eds., National Institute of Standards and Technology, Gaithersburg Md., 20899, https://doi.org/10.18434/T4D303), are ΔH₁°=246 kJ/mol and ΔG₁°=31 kJ/mol. Aromatization alone is indeed disfavored. However, the reaction occurs when coupled with hydrogenation of a suitable hydrogen acceptor. In solventless PE depolymerization, the PE chains themselves serve as an internal hydrogen sink, (see FIG. 6). Using Benson group contributions again, estimated thermodynamic values for C—C bond hydrogenolysis (eq 2) are ΔH₂°=−49 kJ/mol and ΔG₂°=−74 kJ/mol.

Cₘ₊ₙH₂₍ₘ₊ₙ₎₊₂+H₂→CₘH₂ₘ₊₂+CₙH₂ₙ₊₂ (2)

Consequently, aromatization becomes allowed at 280° C. (ΔG°=0) when even 10% of the H₂ generated is consumed in PE hydrogenolysis.

Based on the alkylaromatic yield, the aromatization in Exp. 2 (FIG. 3) generated 0.50 mmol H₂. Over 90% of this H₂ (0.47 mmol) was consumed in reducing the molecular weight of the polymer via hydrogenolysis, making the tandem process thermodynamically favorable. However, the residual H₂ found in the reactor headspace at the end of the reaction (0.11 mmol) exceeds the expected value (0.03 mmol). Therefore, a significant amount of H₂ is generated in other reactions, such as PE dehydrocyclization to give cycloalkanes and tetralins. Both were observed by FD-MS. Their yields are higher than the thermodynamic predictions, which favor aromatics. Some of these more saturated compounds condense outside the autoclave's heated zone where the catalyst is located, thereby preventing their further dehydrogenation.

To explore whether polyethylene is necessary to produce long-chain alkylaromatics by tandem catalytic hydrogenolysis/aromatization, the reaction of n-C₃₀H₆₂ under the same conditions was investigated (Table 5, Exps. S2 and S3). Compared to PE, the n-C₃₀H₆₂ chains experience only half as much hydrogenolysis (consuming just 0.25 mmol H₂ based on FIG. 10), as expected based on the chain length-dependence of hydrogenolysis kinetics (Flaherty, et al., J. Am. Chem. Soc. 135, 18586-18599 (2013)). Molecules in the liquid products have an average chain length of C20, with low alkylaromatic content (ca. 10 mol %). Since hydrogenolysis and aromatization occur in tandem, they occur together. Consequently, the formation of alkylaromatics is greatly enhanced by the use of polyethylene as a feedstock.

TABLE 5
GPC and ¹H NMR characterization of liquid/wax hydrocarbons
recovered from the conversion of triacontane and PEª

| | | | Products | | | | | | |
|---|---|---|---|---|---|---|---|---|---|
| | | Reactor | | | | | | | |
| | | volume | Yield | Mₙ | | | H_{Ar}/ | Hₘₒₙₒ/ | S_{Ar}^{c} |
| Exp. | Reactant | mL | % | g mol⁻¹ | Ð | C_{avg} ^{b} | Hₜₒₜₐₗ | H_{poly} | % |
| S2 | C₃₀H₆₂ | 10 | 70 | — | — | 17 ^{g} | 0.012 | 2.2 | 9 |
| S3 | | 13 | 72 | — | — | 20 ^{g} | 0.016 | 2.1 | 14 |
| S4 | PE | 20^{d} | 66 | 405 | 1.88 | 30 | 0.018 | 2.0 | 26(2) |
| S5 | | 20^{e} | 49 | 497 | 1.87 | 35 | 0.021 | 1.7 | 36(3) |
| S6 | | 90^{f} | 4 | 234 | 1.08 | 17 | 0.017 | 1.1 | 14(1) |
| | | | 46 | 373 | 1.29 | 27 | 0.021 | 1.8 | 27(2) |

^{a}Reaction conditions: 118 mg reactant (triacontane or PE, Mₙ = 1.85 × 10³ g mol⁻¹, Ð = 1.90), 200 mg reduced catalyst (Pt/γ-Al₂O₃, 1.5 wt % Pt), 280° C. for 24 h.
^{b} Calculated as Mₙ/14.
^{c} Selectivity for alkylaromatics (S_{Ar}), as defined above, calculated based on analysis of ¹H NMR spectrum. Uncertainties are propagated from the uncertainty in the average carbon number.
^{d}Reaction in thick-walled glass reactor using fresh catalyst.
^{e}Reaction in thick-walled glass reactor using recycled catalyst.
^{f}Reaction with 1.1 g PE reactant and 1.8 g catalyst at 280° C.. Liquid/wax hydrocarbon products were collected and analyzed separately.
^{g} Calculated based on GC-FID analysis using eq S3, as shown above.

TABLE 6
Benson group increments (Stein and Brown, NIST Chemistry WebBook, NIST Standard Reference Database Number 69,
P.J. Linstrom, W. G. Mallard, Eds., National Institute of Standards and Technology, Gaithersburg MD, 20899,
https://doi.org/10.18434/T4D303) for sub-groups found in linear polyethylene and ortho-substituted dialkylaromatics.

| | | | | Estimated gas-phase increments | | | | | | |
|---|---|---|---|---|---|---|---|---|---|---|
| Group | Chemical | Δ_{f}H_{gas,est} | S_{gas,est} | Cₚ J/mol K at various temperatures (K) | | | | | | |
| label | identity | kJ/mol | J/mol K | 300 | 400 | 500 | 600 | 800 | 1000 | 1500 |
| g₁ | —CH2— | −21.0 | 39.4 | 23.0 | 29.1 | 34.5 | 39.1 | 46.3 | 51.6 | 59.4 |
| g₂ | —CH3 | −42.7 | 127.2 | 25.9 | 32.8 | 39.3 | 45.2 | 54.5 | 61.8 | 73.6 |
| g₃ | C_{AR}—C | 23.0 | −32.1 | 11.2 | 13.1 | 15.4 | 17.4 | 20.8 | 22.8 | 25.0 |
| g₄ | C_{Ar}—H | 14.0 | 48.2 | 13.6 | 18.6 | 22.8 | 26.4 | 31.5 | 35.2 | 40.7 |
| g₅ | Ar—C_{α}H₂— | −20.0 | 39.1 | 24.4 | 31.8 | 37.6 | 41.9 | 48.1 | 52.5 | 57.6 |
| g₆ | Ring strain | 3.0 | −25.0 | 4.6 | 6.3 | 5.4 | 5.0 | 3.8 | 2.9 | −0.4 |
| | correction | | | | | | | | | |
| g_{H} | H₂ | 0.0 | 130.7 | 28.9 | 29.2 | 29.2 | 29.3 | 29.6 | 30.2 | 32.3 |

Although there are far too many individual reactions and products to formulate a precise kinetic model, a simplified model captures the main features of the tandem reaction. It is assumed that the Pt surface is covered with molten PE and/or PE-derived hydrocarbons at all times, and that the hydrogenolysis turnover frequency is constant on sites not occupied by aromatic hydrocarbons. As the latter form, they adsorb more strongly than alkanes (Xu, et al., J. Phys. Chem. 98, 585-593 (1994)), occupying active sites and reducing the hydrogenolysis rate accordingly. Hydrogenolysis is assumed random, i.e., all aliphatic C—C bonds are equally likely to be cleaved (Carter, et al., J. Catal. 20, 223-229 (1971)). The sole adjustable parameter, k/K, is the ratio of the rate constant for hydrogenolysis (k) and the equilibrium constant for competitive adsorption of aromatics and aliphatic chains (K, eq S11). Eq 3 predicts the evolution of the average chain length as a function of the reaction time, initial total carbon amount nc, and total platinum mass m_{Pt}.

$$ln ( 1 - \frac{3}{{M}_{n} ( t )} + \frac{3}{{M}_{n} ( 0 )} ) + ( \frac{3}{{M}_{n} ( t )} - \frac{3}{{M}_{n} ( 0 )} ) = - \frac{k}{K} \frac{{m}_{Pt}}{{n}_{C}} t \qquad ( 3 )$$

The curvefit of eq 3 to an experimental dataset is shown in FIG. 9B, starting with the datapoint at t=1 h (i.e., after the induction period caused by reactor heating). Assuming a preferential binding for aromatics of K=3.2×10⁶ (Xu, et al., J. Phys. Chem. 98, 585-593 (1994)), the hydrogenolysis rate constant k is estimated to be 6.4×10² mol_{C—C} bonds h⁻¹ per g_{Pt} at 280° C.

### Example 3. The Pt/Al₂O₃Catalyst can be Recycled and Reused

Materials and Methods

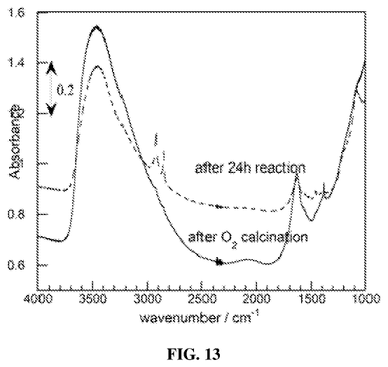

The solid material was recovered from two parallel PE depolymerization reactions conducted at 280° C. for 24 h in mini-autoclave reactors, combined and calcined in flowing O₂ (50 mL/min) at 400° C. for 2 h to remove organic residues. The absence of hydrocarbons was verified by the complete disappearance of the C—H stretching modes in the IR spectrum (FIG. 13). The calcined catalyst was re-reduced in flowing H₂ (4 vol % in Ar, 50 mL/min) at 250° C. for 3 h to give 0.340 g recycled catalyst, which was stored in an Ar-filled glove-box until further use. A portion of the recycled catalyst (0.200 g) was loaded with low molecular weight PE (Mₙ=1.85×10³ g mol⁻¹, Ð=1.90, 0.118 g) into a thick-walled glass reactor. The reaction was conducted at 280° C. for 24 h, as described in Example 1.

Results

The Pt/γ-Al₂O₃ catalyst can be recovered and recycled. In the spent catalyst, the Pt NPs had increased in size slightly, from 0.8 to 1.2 nm (FIG. 1B). Upon reuse, the yield of the soluble liquid/wax fraction decreased slightly, although the alkylaromatic selectivity increased (Table 5, Exp. S4 and S5). In a preliminary scale-up attempt, the amount of PE was increased nearly ten-fold (to 1.1 g), while maintaining the same PE:Pt ratio and reaction conditions. After 24 h, 0.56 g of a liquid product (M_{w}=483 g mol⁻¹, Ð=1.29) with 27 mol % aromatic content was obtained (Table 5, Exp. S6).

### Example 4. The Pt/Al₂O₃Catalyst Converts Commercial Low-Density Polyethylene and High-Density Polyethylene to Alkylaromatics at Low Temperatures

Materials and Methods

A low molecular weight polyethylene (Lot SKU-427772, Mₙ=1.85×10³ g mol⁻¹; M_{w}=3.52×10³ g mol⁻¹; Ð=1.9) was purchased from Sigma Aldrich. Low density polyethylene (LDPE) was obtained from a discarded plastic packaging bag (Mₙ=12.8×10³ g mol⁻¹; M_{w}=94.5×10³ g mol⁻¹; Ð=7.4), and was cut into mm-size pieces before use. High density polyethylene (HDPE) was the cap of a disposable PET water bottle (Mₙ=14.8×10³ g mol⁻¹; M_{w}=53.5×10³ g mol⁻¹; Ð=3.6). It was cut into mm-size pieces, frozen in liquid N₂, and the particle size was further reduced in a coffee-grinder before use. The reaction was conducted larger autoclave reactor (Parr Series 5000 Multiple Reactor System, internal volume ca. 100 mL, stainless steel), as described in Example 1.

Results

To investigate how a tandem catalytic process could be deployed to convert waste polyethylene without large energy input, solvent-free depolymerization of two different commercial grades of PE: an LDPE plastic bag (M_{w}=9.45×10⁴ g mol⁻¹, Ð=7.37), and an HDPE water bottle cap (M_{w}=5.35×10⁴ g mol⁻¹, Ð=3.61) was performed. These higher molecular weight polymers behaved similarly to the low molecular weight polyethylene, giving liquid/wax products with an average carbon number of ca. C₃₀. After 24 h at 280° C., the overall liquid yields were 69 and 55 wt % for LDPE and HDPE, respectively (FIG. 3, Exps. 4-5), with alkylaromatic selectivities of ca. 44 and 50 mol % (Table 2A, Exps. 4-5). Thus the extent of depolymerization is slightly lower in the same reaction time. For these higher molecular weight polyethylenes, the batch process generates its own highly viscous solvent as depolymerization proceeds. Recycling some of the alkylaromatic liquids to serve as solvent for the next batch may accelerate the reaction by facilitating mass and heat transport.

Shorter residence times should also improve the selectivity for monoaromatic hydrocarbons relative to naphthalenes, etc., and suppress the already low gas yields even further. Alkylbenzene selectivity may be further improved by active control of P(H₂), which is high enough to promote PE hydrogenolysis, but low enough to suppress aromatic hydrogenation. The alkylbenzenes with their linear side-chains could be sulfonated to produce biodegradable surfactants. This type of commodity polymer upcycling leading to chemical products that displace fossil carbon-based feedstocks, while simultaneously incentivizing better management of plastic waste and recovering considerable material value that can be recirculated into the global economy.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A process for upcycling a waste material, wherein the waste material comprises a hydrocarbon polymer, optionally more than one hydrocarbon polymer, comprising
(i) feeding the waste material into a reactor,
wherein the reactor comprises a catalyst therein,
wherein the catalyst comprises a transition metal; and
(ii) operating the reactor at a sufficient temperature for a sufficient period of time to convert the hydrocarbon polymer to a product in the form of a liquid and/or a wax comprising an alkylaromatic compound, optionally more than one alkylaromatic compound,
wherein the mol % of the alkylaromatic compound, optionally the total mol % of the more than one alkylaromatic compound, in the product is at least 50 mol %.

2. The process of claim 1, wherein the hydrocarbon polymer is polyethylene, polypropylene, polystyrene, a copolymer of polyethylene and polypropylene, or acrylonitrile butadiene styrene.

3. The process of claim 1, wherein the alkylaromatic compound contains from 10 to 50 carbon atoms, from 12 to 50 carbon atoms, from 15 to 50 carbon atoms, from 15 to 25 carbons, from 20 to 40 carbons, or from 20 to 35 carbon atoms.

4. The process of claim 1, wherein the alkylaromatic compound has a weight average molecular weight (Mw) in a range from 150 g mol⁻¹ to 800 g mol⁻¹, from 200 g mol⁻¹ to 800 g mol⁻¹, from 200 g mol⁻¹ to 600 g mol⁻¹, from 200 g mol⁻¹ to 500 g mol⁻¹, from 300 g mol⁻¹ to 800 g mol⁻¹, from 300 g mol⁻¹ to 600 g mol⁻¹, or from 300 g mol⁻¹ to 500 g mol⁻¹.

5. The process of claim 1, wherein the product further comprises an olefin.

6. The process of claim 1, wherein the alkylaromatic compound is a dialkylbenzene, an alkyltetralin, a dialkylnaphthalene, an alkylanthracene, an alkylphenanthrene, a partially hydrogenated alkyanthracene, or a partially hydrogenated alkylphenathrene, or a combination thereof.

7. The process of claim 6, wherein the dialkylbenezene has a structure of Formula (I): 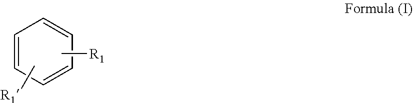
wherein R₁ and R₁′ are independently a C₁-C₂₀ alkyl group, a C1-C₁₅ alkyl group, a C₁-C₁₂ alkyl group, a C₁-C₁₀ alkyl group, or a C₁-C₅ alkyl group, and
wherein the sum of the carbon atoms in R₁ and R₁′ is at least 10.

8. The process of claim 6, wherein the dialkylnaphthalene has a structure of Formula (II) 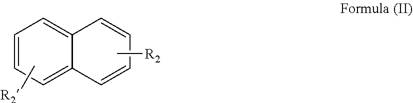
wherein R₂ and R₂′ are independently a C₁-C₂₀ alkyl group, a C1-C₁₅ alkyl group, a C₁-C₁₂ alkyl group, a C₁-C₁₀ alkyl group, or a C₁-C₅ alkyl group, and
wherein the sum of the carbon atoms in R₂ and R₂′ is at least 4, optionally at least 5.

9. The process of claim 1, wherein the transition metal is selected from the group consisting of platinum, palladium, ruthenium, iridium, rhenium, rhodium, iron, cobalt, nickel, copper, molybdenum, and tungsten.

10. The process of claim 1, wherein the catalyst comprises more than one transition metal, wherein each of the transition metals is selected from the group consisting of platinum, palladium, ruthenium, iridium, rhenium, rhodium, iron, cobalt, nickel, copper, molybdenum, and tungsten.

11. The process of claim 1, wherein the catalyst comprises more than one transition metal, wherein each of the transition metals is selected from the group consisting of platinum, palladium, ruthenium, iridium, rhodium, iron, cobalt, nickel, copper, molybdenum, and tungsten; and wherein the catalyst further comprises a second metal, wherein the second metal is different from each of the transition metals, and wherein the second metal is selected from the group consisting of rhenium, tin, lead, tungsten, molybdenum, chromium, manganese, and zinc, or a combination thereof

12. The process of claim 1, wherein the catalyst is a metal, a mixture of two or more metals comprising the transition metal, a metal oxide of the transition metal, or a metal carbide of the transition metal, or a combination thereof.

13. The process of claim 1, wherein the catalyst is dispersed on a surface of a first support and wherein the catalyst is in the form of atoms, nanoclusters, or nanoparticles, or a combination thereof.

14. The process of claim 13, wherein the catalyst is platinum nanoparticles, wherein the first support is an aluminum oxide support or silica-aluminum oxide support, and wherein the platinum nanoparticles are dispersed on the surface of the aluminum oxide support or silica-aluminum oxide support.

15. The process of claim 13, further comprising a second support, wherein the first support having the catalyst dispersed thereon is mixed with the second support and the second support is an acidic support.

16. The process of claim 15, wherein the second support is a halogenated alumina support, optionally a fluorinated alumina support.

17. The process of claim 13, wherein the total weight loading of the metal is in a range from 0.1 wt % to 10 wt %, from 0.1 wt % to 8 wt %, from 0.1 wt % to 5 wt %, from 0.1 wt % to 1 wt %, from 0.5 wt % to 10 wt %, from 0.5 wt % to 8 wt %, from 0.5 wt % to 5 wt %, from 1 wt % to 10 wt %, from 1 wt % to 8 wt %, or from 1 wt % to 5 wt % of the total weight of the catalyst and the first support or the total weight of the catalyst and the first and second support.

18. The process of claim 15, wherein the weight percentage of the second support is in a range from 2% to 90%, from 2% to 50%, from 2% to 35%, from 10% to 90%, from 10% to 80%, from 10% to 70%, from 10% to 60%, from 10% to 50%, from 10% to 30%, from 15% to 40%, from 20% to 50%, from 20% to 40%, or from 20% to 35% of the total weight of the catalyst and the first and second support.

19. The process of claim 1, wherein during step (ii), the reactor is operated at a temperature of up to 500° C., up to 450° C., up to 400° C., up to 350° C., up to 320° C., up to 300° C., up to 290° C., between 250° C. and 500° C., between 250° C. and 450° C., between 250° C. and 400° C., between 300° C. and 500° C., between 320° C. and 500° C., between 300° C. and 450° C., between 320° C. and 450° C., between 300° C. and 400° C., between 300° C. and 360° C., between 250° C. and 350° C., between 250° C. and 350° C., between 250° C. and 320° C., or between 250° C. and 300° C.

20. The process of claim 1, wherein during step (ii), the reactor is operated for a period of time of up to 6 hours, up to 5.5 hours, up to 5 hours, up to 4.5 hours, up to 4 hours, up to 3.5 hours, up to 3 hours, up to 2.5 hours, up to 2 hours, up to 1.5 hours, up to 1 hour, up to 55 minutes, up to 50 minutes, up to 45 minutes, up to 40 minutes, up to 35 minutes, up to 30 minutes, up to 25 minutes, up to 20 minutes, up to 15 minutes, up to 10 minutes, up to 5 minutes, in a range from 5 minutes to 6 hours, from 5 minutes to 5.5 hours, from 5 minutes to 5 hours, from 5 minutes to 4.5 hours, from 5 minutes to 4 hours, from 5 minutes to 3.5 hours, from 5 minutes to 3 hours, from 5 minutes to 2.5 hours, from 5 minutes to 2 hours, from 5 minutes to 1.5 hours, from 1 minute to 1 hour, from 5 minutes to 1 hour, from 10 minutes to 1 hour, from 15 minutes to 1 hour, from 20 minutes to 1 hour, from 30 minutes to 1 hour, from 1 minute to 55 minutes, from 1 minute to 50 minutes, from 1 minute to 45 minutes, from 1 minute to 40 minutes, from 1 minute to 35 minutes, or from 1 minute to 30 minutes.

21. The process of claim 1, wherein following steps (i) and (ii), the mol % of the alkylaromatic compound, optionally the total mol % of the more than one alkylaromatic compound, in the product is at least 30 mol %, at least 35 mol %, at least 40 mol %, at least 45 mol %, at least 50 mol %, at least 55 mol %, at least 60 mol %, at least 65 mol %, at least 70 mol %, at least 75 mol %, at least 80 mol %, at least 85 mol %, at least 90 mol %, at least 95 mol %, between 30 mol % and 95 mol %, between 35 mol % and 95 mol %, between 50 mol % and 95 mol %, or between 60 mol % and 90 mol %.

22. The process of claim 1, wherein following steps (i) and (ii), the liquid and/or wax product has a yield of at least 70 wt %, at least 75 wt %, at least 80 wt %, at least 85 wt %, at least 90 wt %, at least 95 wt %, between 70 wt % and 95 wt %, or between 75 wt % and 95 wt %.

23. The process of claim 1, wherein following steps (i) and (ii), the alkylaromatic compound has a yield of at least 40 wt %, at least 45 wt %, at least 50 wt %, at least 55 wt %, at least 60 wt %, at least 65 wt %, at least 70 wt %, at least 75 wt %, at least 80 wt %, between 40 wt % and 85 wt %, between 45 wt % and 85 wt %, between 50 wt % and 85 wt %, between 55 wt % and 85 wt %, or between 60 wt % and 85 wt.

24. The process of claim 1, wherein the hydrocarbon polymer is a high density polyethylene polymer or a low density polyethylene polymer, or a combination thereof.

25. The process of claim 1, wherein following steps (i) and (ii), the distribution of the molecular weight of the alkylaromatic compound has a dispersity of less than 3.5, less than 3.0, less than 2.5, less than 2.0, less than 1.5, less than 1.3, less than 1.2, between 1 and 1.5, between 1 and 1.4, between 1 and 1.3, or between 1 and 1.2, between 1.1 and 1.5, between 1.1 and 1.4, between 1.1 and 1.3, or between 1.1 and 1.2.

26. The process of claim 1, further comprising a step of (iii) processing the waste to a suitable form prior to step (i).

27. The process of claim 26, wherein the waste is in the form of solid waste, and wherein step (iii) includes shredding, cutting, and/or grinding the waste hydrocarbon polymer to small parts.

28. The process of claim 1, wherein the waste is in the form of solid waste, and wherein the process further comprises, prior to step (i), a step of dissolving the solid waste in a solvent, optionally in a hydrocarbon solvent.

29. The process of claim 1, further comprising a step of (iv) cooling the reactor to room temperature after step (ii).

30. The process of claim 1, further comprising a step of (v) recycling the catalyst after step (ii).

31. The process of claim 14, further comprising a second support, wherein the first support having the catalyst dispersed thereon is mixed with the second support and the second support is an acidic support.

32. The process of claim 31, wherein the second support is a halogenated alumina support, optionally a fluorinated alumina support.

33. The process of claim 32, wherein the weight percentage of the halogen, optionally the fluorine, in the second support is in a range from 0.1% to 5%, from 0.5% to 3%, from 0.5% to 2%, from 0.5% to 1.5%, optionally about 0.7% or about 1.3%.

34. The process of claim 32, wherein the weight percentage of the second support is in a range from 2% to 35%, from 5% to 35%, from 10% to 35%, or from 10% to 30% of the total weight of the catalyst and the first and second support.

35. The process of claim 32, wherein during step (ii), the reactor is operated at a temperature of up to 350° C., up to 320° C., up to 300° C., in a range from 250° C. to 350° C., from 250° C. to 350° C., from 250° C. to 320° C., or from 250° C. to 300° C., optionally about 280° C.

36. The process of claim 32, wherein during step (ii), the reactor is operated for a period of time of up to 2.5 hours, up to 2 hours, up to 1.5 hours, up to 1 hour, in a range from 5 minutes to 2.5 hours, from 5 minutes to 2 hours, from 5 minutes to 1.5 hours, from 1 minute to 1 hour, from 5 minutes to 1 hour, from 10 minutes to 1 hour, from 15 minutes to 1 hour, from 20 minutes to 1 hour, or from 30 minutes to 1 hour.

37. The process of claim 32, wherein following steps (i) and (ii), the mol % of the alkylaromatic compound, optionally the total mol % of the more than one alkylaromatic compound, in the product is at least 25 mol %, at least 30 mol %, at least 40 mol %, in a range from 25 mol % to 95 mol %, from 30 mol % to 95 mol %, or from 40 mol % to 95 mol %.

38. The process of claim 32, wherein following steps (i) and (ii), the liquid and/or wax product has a yield of at least 50 wt %, at least 55 wt %, at least 60 wt %, at least 65 wt %, in a range from 50 wt % to 95 wt %, or from 60 wt % to 95 wt %.

39. A composition comprising an alkylaromatic compound, optionally more than one alkylaromatic compound, wherein the composition is in the form of a liquid and/or wax, and wherein the composition is produced by the process of any one of claims 1 to 38.

40. The process of claim 16, wherein the weight percentage of the halogen, optionally the fluorine, in the second support is in a range from 0.1% to 5%, from 0.5% to 3%, from 0.5% to 2%, from 0.5% to 1.5%, optionally about 0.7% or about 1.3%.

41. The process of claim 16, wherein the weight percentage of the second support is in a range from 2% to 35%, from 5% to 35%, from 10% to 35%, or from 10% to 30% of the total weight of the catalyst and the first and second support.

42. The process of claim 16, wherein during step (ii), the reactor is operated at a temperature of up to 350° C., up to 320° C., up to 300° C., in a range from 250° C. to 350° C., from 250° C. to 350° C., from 250° C. to 320° C., or from 250° C. to 300° C., optionally about 280° C.

43. The process of claim 16, wherein during step (ii), the reactor is operated for a period of time of up to 2.5 hours, up to 2 hours, up to 1.5 hours, up to 1 hour, in a range from 5 minutes to 2.5 hours, from 5 minutes to 2 hours, from 5 minutes to 1.5 hours, from 1 minute to 1 hour, from 5 minutes to 1 hour, from 10 minutes to 1 hour, from 15 minutes to 1 hour, from 20 minutes to 1 hour, or from 30 minutes to 1 hour.

44. The process of claim 16, wherein following steps (i) and (ii), the mol % of the alkylaromatic compound, optionally the total mol % of the more than one alkylaromatic compound, in the product is at least 25 mol %, at least 30 mol %, at least 40 mol %, in a range from 25 mol % to 95 mol %, from 30 mol % to 95 mol %, or from 40 mol % to 95 mol %.

45. The process of claim 16, wherein following steps (i) and (ii), the liquid and/or wax product has a yield of at least 50 wt %, at least 55 wt %, at least 60 wt %, at least 65 wt %, in a range from 50 wt % to 95 wt %, or from 60 wt % to 95 wt %.

46. A composition comprising an alkylaromatic compound, optionally more than one alkylaromatic compound, wherein the composition is in the form of a liquid and/or wax, and wherein the composition is produced by the process of any one of claims 40 to 45.
